(19) United States
(12) Patent Application Publication
(10) Pub. No.: US 2022/0218001 A1
(43) Pub. Date: Jul. 14, 2022

(54) REDOX ENZYMES IN ANIMAL FEED COMPOSITIONS

(71) Applicant: Novozymes A/S, Bagsvaerfd (DK)

(72) Inventors: Ming Li, Beijing (CN); Marianne Thorup Cohn, Nordhavn (DK); Kirk Matthew Schnorr, Holte (DK); Elodie Bacou, Kaiseraugst (SE); Luis Fernando Romero Millan, Kaiseraugst (SE); Maria Walsch, Kaiseraugst (SE); Tianqi Sun, Beijing (CN); Ye Liu, Beijing (CN); Lan Tang, Beijing (CN); Estefania Perez Calvo, Kaiseraugst (SE); Peter Wagner, Kaiseraugst (SE)

(73) Assignee: Novozymes A/S, Bagsvaerd (DK)

(21) Appl. No.: 17/441,731

(22) PCT Filed: Apr. 5, 2020
(86) PCT No.: PCT/CN2020/083408

§ 371 Date: Sep. 22, 2021

(30) Foreign Application Priority Data
Apr. 5, 2019 (DK) PA 2019 00423
Aug. 16, 2019 (CN) PCT/CN2019/101088

(51) Int. Cl. A23K 20/189; C12N 9/02; C12N 9/08; A23K 20/147; A61K 38/44; A61P 37/02; A61P 29/00

(52) U.S. Cl. CPC A23K 20/189; C12N 9/0089; C12Y 115/01001; C12N 9/0065; C12Y 111/01006; A61P 29/00; A23K 20/147; A61K 38/44; A61K 38/446; A61P 37/02; C12Y 111/01021

(57) ABSTRACT

Novel superoxide dismutases of fungal origin are active, gastric stable and thermal stable, and effective for use in animal feed additives. The use of superoxide dismutase in animal feed improve animal growth, animal health and intestinal health of animals.

This application contains a Sequence Listing in computer readable form, which is incorporated herein by reference.

### FIELD OF THE INVENTION

The present invention also relates to new polypeptides of fungal origin having superoxide dismutase activity and to animal feed additive or animal feed comprising superoxide dismutase of fungal origin. The present invention relates to the use of polypeptides of fungal origin having superoxide dismutase activity for
improving one or more performance parameters, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU),
improving or enhancing immune response and/or reducing inflammation and/or for the modulation of the gut flora in an animal, and
reducing or eliminating the use of antibiotics administered to animal feed.

### BACKGROUND OF THE INVENTION

Superoxide dismutase (SOD, EC 1.15.1.1) is an enzyme that alternately catalyzes the dismutation (or partitioning) of the superoxide (O₂⁻) radical into either ordinary molecular oxygen (O₂) or hydrogen peroxide (H₂O₂). Superoxide is produced as a by-product of oxygen metabolism and, if not regulated, causes many types of cell damage. Thus, SOD is an important antioxidant defense in nearly all living cells exposed to oxygen. SODs are used in the pharmaceutical, cosmetic, food, and environmental protection industries due to their excellent antioxidant properties. Historically, SODs were isolated from animal or plant sources, but the microbial sources organisms can be easily induced and cultivated on a large scale

SODs naturally occur in many organisms such as plants, insects, birds, reptiles and mammals. Four types of SODs have been reported according to their metal cofactors: manganese SOD (Mn-SOD), iron SOD (Fe-SOD), copper/zinc SOD (Cu/Zn-SOD), and nickel SOD (Ni-SOD)₂.

Commercially available mammalian (bovine) SOD (Sigma), a Cu/Zn SOD, although having a high activity at neutral pH or at its optimal pH of 7.8, was found to lose 65% of its activity under gastric stability studies. According to its datasheet, its pH range is 7.6-10.5. Commerically available bacterial (from E. coli) Mn-SOD (S5639 from Sigma) was found to lose 57% of its activity under gastric stability studies.

Surprisingly, the inventors of the present invention found that fungal SODs are gastric stable and provide beneficial effects when used in an animal feed or animal feed additive, alone or in combination with a catalase, to improve one or more performance parameters, enhance immune response and reduce inflammation in an animal and thus lead to many advantages such as improving animal health and/or animal performance, and/or reducing feeding cost.

### SUMMARY OF THE INVENTION

The invention is directed, at least in part, to novel polypeptides of fungal origin having superoxide dismutase activity and directed to an animal feed additive comprising a polypeptide of fungal origin having superoxide dismutase activity. A further aspect is directed to an animal feed additive comprising at least one and no more than two enzyme classes, wherein the at least one enzyme class is a superoxide dismutase of fungal origin and the optional second enzyme class is a catalase.

The novel polypeptides are isolated fungal polypeptides having superoxide dismutase activity are selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

Alternatively defined, the novel polypeptides having superoxide dismutase activity are isolated fungal polypeptides selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

The animal feed additive comprising one or more polypeptides of fungal origin having superoxide dismutase (SOD) activity wherein the polypeptide having superoxide dismutase activity is selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.

The invention is furthermore directed to an animal feed or animal feed additive comprising polypeptides of fungal origin having superoxide dismutase activity. The present invention relates to the use of polypeptides of fungal orgin having superoxide dismutase activity for the maintenance of general health or intestinal health or the prophylactic or preventative treatment or early treatment of symptoms associated to direct and indirect environmental stresses. Direct stresses result from for example temperature, solar radiation, humidity, rainfall and wind velocity. Indirect stresses comprise those which affect the supply and availability of food (nutritional stress) and are determined by climatic and other factors which affect the growth of the plants that animals eat.

The present invention relates to an animal feed or animal feed additive comprising one or more polypeptides having SOD activity, typically to an animal feed additive comprising a superoxide dismutase of fungal origin and optionally of a catalase.

Alternatively defined, the present invention relates to an animal feed additive comprising at least one and no more than two enzyme classes, wherein the at least one enzyme class is a superoxide dismutase of fungal origin and the optional second enzyme class is a catalase. An aspect of the invention is directed to an animal feed additive comprising an enzyme component, wherein the enzyme component comprises all of the enzymes of the additive and consists of a superoxide dismutase of fungal origin and optionally of a catalase. The animal feed additive as defined above suitably may further comprise of one or more polypeptides having catalase activity and/or one or more vitamins. A further aspect of the invention is directed to the use of an animal feed additive as defined herein for replacing or partially replacing antibiotics in animal feed.

The invention also relates to a method of
improving one or more performance parameters in an animal comprising administering to the animal an animal feed or animal feed additive comprising one or more polypeptides having superoxide dismutase activity, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).
improving or enhancing immune response and/or reducing inflammation and/or for the modulation of the gut flora in an animal comprising administering to the animal an animal feed or animal feed additive comprising one or more polypeptides having superoxide dismutase activity.
reducing or eliminating the use of antibiotics administered to animal feed, comprising administering to the animal an animal feed or animal feed additive comprising of one or more polypeptides having superoxide dismutase activity.

An aspect of the invention is directed to a method of improving or enhancing immune response and/or reducing inflammation and/or for the modulation of the gut flora in an animal comprising administering to the animal the animal feed additive as defined herein or an animal feed comprising the animal feed additive as defined herein.

An aspect of the invention is directed to a method of reducing or eliminating the use of antibiotics administered to an animal feed, comprising administering to the animal the animal feed additive as defined herein or an animal feed comprising the animal feed additive as defined herein.

A further aspect of the invention is directed to the prophylactic care or management, reduction or prevention of oxidative stress in a monogastric animal comprising administrating to said animal superoxide dismutase and optionally catalase. Oxidative stress is a disturbance between antioxidant/oxidant status in favor of excessive generation, or slower removal of free radicals, such as reactive oxygen species (ROS). Excessive ROS content leads to damage of proteins, lipids and nucleic acids, with consequent loss of their biological functions and subsequent tissue injury. Oxidative stress has been linked to initiation and progression of several infectious diseases. Accordingly, a further aspect of the invention is the prophylactic care or management of infectious diseases in monogastric animal comprising administrating to said animal superoxide dismutase and optionally catalase. A related aspect is directed to a use of an animal feed additive as defined herein as an antioxidant, preferably in animal feed and feed premixes.

The administration is suitably by means of feeding said animal a feed additive or feed comprising an enzyme component, wherein the enzyme component comprises all of the enzymes of the additive and consists of a superoxide dismutase and optionally of a catalase.

The invention is further directed to an animal feed comprising one or more protein sources and one or more energy sources characterised in that the animal feed further comprises one or more polypeptides having catalase activity and/or one or more polypeptides having superoxide dismutase (SOD) activity, wherein the animal feed
a. reduces FCR by between 1 to 10%; or
b. increases average daily WG by between 1 to 10%; or
c. increases average daily feed intake by between 1 to 10%; or
d. any combinations of a.-c.;

when fed to the animal as compared to animals fed a feed composition without the polypeptides.

The invention is further directed to an animal feed comprising one or more protein sources and one or more energy sources characterised in that the animal feed further comprises one or more polypeptides having superoxide dismutase (SOD) activity, wherein the animal feed
a. improves or enhances immune response; or
b. reduces inflammation;

when fed to the animal as compared to animals fed a feed composition without the polypeptides; and

said feed optionally further comprising one or more polypeptides having catalase activity.

Alternatively stated, the invention is, in a further aspect, directed to an animal feed comprising one or more protein sources and one or more energy sources characterised in that the animal feed further comprises one or more polypeptides having catalase activity and/or one or more polypeptides having superoxide dismutase (SOD) activity, wherein the animal feed
a. improves or enhances immune response; or
b. reduces inflammation;

when fed to the animal as compared to animals fed a feed composition without the polypeptides.

### BRIEF DESCRIPTION OF THE FIGURES

FIG. 1: illustrates the body weight gain (BWG) in poultry from days 0-35. The graphs illustrate the negative impact of heat stress on BWG. In the non-heat stressed animals, the effect of the SOD/CAT combination outperformed or equaled the positive control (Vit E/Se). In heat stressed animals, both the Vit E/Se positive control and the SOD/CAT combination improved BWG.

FIG. 2: illustrates the Conversion Ratio (FCR) in poultry from days 0-35. The graphs illustrate the negative impact (increase) of heat stress on Feed Conversion Ratio (FCR). In the non-heat stressed animals, the effect of the SOD/CAT combination equaled the positive control (Vit E/Se).

FIG. 3: illustrates the Mortality in poultry from days 0-35. The graphs illustrate the negative impact (increase) of heat stress on Mortality. In heat stressed animals, both the Vit E/Se positive control and the SOD/CAT combination reduced Mortality.

FIG. 4: illustrates the body weight gain (BWG) from days 27-35. The graphs illustrate the negative impact of heat stress on BWG. In the non-heat stressed animals, the effect of the SOD/CAT combination outperformed or equaled the positive control (Vit E/Se).

FIG. 5: illustrates the Conversion Ratio (FCR) from days 27-35. The graphs illustrate the negative impact (increase) of heat stress on Feed Conversion Ratio (FCR). In the non-heat stressed animals, the effect of the SOD/CAT combination equaled the positive control (Vit E/Se).

FIG. 6: illustrates the Mortality from days 27-35. The graphs illustrate the negative impact (increase) of heat stress on Mortality. In heat stressed animals, both the Vit E/Se positive control and the SOD/CAT combination reduced Mortality.

FIG. 7: illustrates the body weight gain (BWG) from days 0-7. In heat stressed animals, both the Vit E/Se positive control and the SOD/CAT combination improved BWG.

FIG. 8: illustrates the Conversion Ratio (FCR) from days 0-7. In heat-stressed animals, the effect of the SOD/CAT combination equaled the positive control (Vit E/Se). In heat stressed animals, both the Vit E/Se positive control and the SOD/CAT combination improved (reduced) FCR.

FIG. 9: illustrates that SOD and CAT is associated with an increase in the expression of liver GPx (pre-heat stress) protein (d 27).

FIG. 10: illustrates that SOD and CAT is associated with a reduction in the expression of Heat Shock Protein HSP70 in the jejunum (d28) The reduction in Heat Shock Protein is indicative od reduced inflammation.

FIG. 11: illustrates that SOD and CAT is associated with a reduction in the expression of Heat Shock Protein HSP70 in the liver (d35). The reduction in Heat Shock Protein is indicative od reduced inflammation.

FIG. 12: illustrates that SOD and CAT is associated with a reduction in the expression of liver iNOS prior to heat stress challenge (d27). Liver iNOS is a mediator of inflammation.

FIG. 13: illustrates that SOD and CAT is associated with a reduction in the expression of liver IL-10 during heat stress (d 35), which qualifies as inflammatory marker.

FIG. 14: illustrates that SOD and CAT is associated with a reduction in the expression of liver TNF alpha during heat stress (d 35), which qualifies as inflammatory marker.

FIG. 15: illustrates that SOD and CAT is associated with a reduction in the expression of liver INF gamma during heat stress (d 35), which qualifies as inflammatory marker.

FIG. 16: shows the basal diet for the piglets of the study

FIG. 17: illustrates the Conversion Ratio (FCR) in piglets from days 0-42. There was significant difference observed in growth performance between positive and negative control. Catalase alone significantly improved the FCR. SOD alone significantly improved the FCR. The combination of the SOD and Catalase allowed for lower FCR or equally low FCR at lower doses. Combining SOD & CAT or feeding CAT and SOD alone gave similar performance to the antibiotic treatment and significantly better than NC.

FIG. 18: illustrates the body weight gain (BWG) in a second piglet trail from days 0-42. There was significant difference observed in growth performance between positive and negative control.

FIG. 19: illustrates the Conversion Ratio (FCR) in the piglets of the second piglet trail from over the period of d0-d42. In particular, inclusion of SOD+CAT (independently of the candidate B or C) showed significant improvement on FCR performance.

### OVERVIEW OF SEQUENCE LISTING

SEQ ID NO 1 is the amino acid sequence of a mature polypeptide having superoxide dismutase (SOD) activity from Trichoderma reesei comprising 287 amino acid residues.

SEQ ID NO 2 is the amino acid sequence of a mature polypeptide having superoxide dismutase (SOD) activity from Aspergillus versicolor comprising 186 amino acid residues

SEQ ID NO 3 is the amino acid sequence of a mature polypeptide having superoxide dismutase (SOD) activity from Aspergillus deflectus comprising 138 amino acid residues.

SEQ ID NO 4 is the amino acid sequence of a mature polypeptide having superoxide dismutase (SOD) activity from Aspergillus egyptiacus comprising 188 amino acid residues wherein residues 1 to 19 make up the signal peptide and residues 47 to 179 make up the SOD.

SEQ ID NO 5 is a polypeptide coding sequence of the polypeptide of SEQ ID NO: 1.

SEQ ID NO 6 is the amino acid sequence of a mature polypeptide having catalase activity from Aspergillus niger comprising 714 amino acid residues.

SEQ ID NO 7 is the amino acid sequence of a mature polypeptide having catalase activity from Aspergillus niger comprising 730 amino acid residues. SEQ ID NO 7 is sold under the tradename Catazyme™.

SEQ ID NO 8 is a polypeptide coding sequence from Aspergillus niger of the polypeptide of SEQ ID NO: 7.

SEQ ID NO 9 is the amino acid sequence of a mature polypeptide having superoxide dismutase (SOD) activity available from Westerdykella sp. AS85-2.

SEQ ID NO 10 is the amino acid sequence of a mature polypeptide having superoxide dismutase (SOD) activity available from Aspergillus sp. XZ2669.

SEQ ID NO 11 is the amino acid sequence of a mature polypeptide having superoxide dismutase (SOD) activity from Preussia terricola.

SEQ ID NO 12 is the amino acid sequence of a mature polypeptide having superoxide dismutase (SOD) activity available from Kionochaeta sp.

SEQ ID NO 13 is the amino acid sequence of a mature polypeptide having superoxide dismutase (SOD) activity available from Kionochaeta sp.

SEQ ID NO 14 is the amino acid sequence of a mature polypeptide having superoxide dismutase (SOD) activity available from Metapochonia bulbillosa.

SEQ ID NO 15 is the amino acid sequence of a mature polypeptide having superoxide dismutase (SOD) activity available from Xylomelasma sp. XZ0718.

SEQ ID NO 16 is the amino acid sequence of a mature polypeptide having superoxide dismutase (SOD) activity available from Preussia flanaganii.

SEQ ID NO 17 is the amino acid sequence of a mature polypeptide having superoxide dismutase (SOD) activity) available from Cladobotryum sp.

SEQ ID NO 18 is a full-length amino acid sequence from the is the cDNA sequence of SEQ ID NO 98 from Cladobotryum sp. The signal start is amino acid residue 1 and the signal stop is amino acid residue 22.

SEQ ID NO 19 is the amino acid sequence of a mature polypeptide having superoxide dismutase (SOD) activity available from Westerdykella sp-46156.

SEQ ID NO 20 is the amino acid sequence of a mature polypeptide having superoxide dismutase (SOD) activity available from Trichoderma hamatum.

SEQ ID NO 21 is the amino acid sequence of a mature polypeptide having superoxide dismutase (SOD) activity available from Mycothermus thermophilus.

SEQ ID NO 22 is the amino acid sequence of a mature polypeptide having superoxide dismutase (SOD) activity available from Cephalotrichiella penicillate.

SEQ ID NO 23 is the amino acid sequence of a mature polypeptide having superoxide dismutase (SOD) activity available from Chaetomium megalocarpum.

SEQ ID NO 24 is the amino acid sequence of a mature polypeptide having superoxide dismutase (SOD) activity available from Chaetomium thermophilum var. thermophilum.

SEQ ID NO 25 is the amino acid sequence of a mature polypeptide having superoxide dismutase (SOD) activity available from Humicola hyalothermophila.

SEQ ID NO 26 is the amino acid sequence of a mature polypeptide having superoxide dismutase (SOD) activity available from Subramaniula anamorphosa.

SEQ ID NO 27 is the amino acid sequence of a mature polypeptide having superoxide dismutase (SOD) activity available from Sphingobacterium sp. T2.

SEQ ID NO 28 is the amino acid sequence of a mature polypeptide having superoxide dismutase (SOD) activity available from Trichoderma rossicum.

SEQ ID NO 29 is the amino acid sequence of a mature polypeptide having superoxide dismutase (SOD) activity available from Trichoderma lixii.

SEQ ID NO 30 is the amino acid sequence of a mature polypeptide having superoxide dismutase (SOD) activity available from Trichoderma sp-54723.

SEQ ID NO 31 is the amino acid sequence of a mature polypeptide having superoxide dismutase (SOD) activity available from Aspergillus niveus.

SEQ ID NO 32 is the amino acid sequence of a mature polypeptide having superoxide dismutase (SOD) activity available from Aspergillus templicola.

SEQ ID NO 33 is the amino acid sequence of a mature polypeptide having superoxide dismutase (SOD) activity available from Pochonia chlamydosporia var. spinulospora.

SEQ ID NO 34 is the amino acid sequence of a mature polypeptide having superoxide dismutase (SOD) activity available from Trichoderma sp-44174.

SEQ ID NO 35 is a full-length amino acid sequence from the is the cDNA sequence of SEQ ID NO 138 from Trichoderma sp-44174. The signal start is amino acid residue 1 and the signal stop is amino acid residue 19.

SEQ ID NO 36 is the amino acid sequence of a mature polypeptide having superoxide dismutase (SOD) activity available from Trichoderma rossicum.

SEQ ID NO 37 is a full-length amino acid sequence from the cDNA sequence of SEQ ID NO 139 from Trichoderma rossicum. The signal start is amino acid residue 1 and the signal stop is amino acid residue 19.

SEQ ID NO 38 is the amino acid sequence of a mature polypeptide having superoxide dismutase (SOD) activity available from Trichoderma sp-54723.

SEQ ID NO 39 is the amino acid sequence of a mature polypeptide having superoxide dismutase (SOD) activity available from Trichoderma sp-44174.

SEQ ID NO 40 is the amino acid sequence of a mature polypeptide having superoxide dismutase (SOD) activity available from Metapochonia suchlasporia.

SEQ ID NO 41 is the amino acid sequence of a mature polypeptide having superoxide dismutase (SOD) activity available from Metarhizium marquandii.

SEQ ID NO 42 is the amino acid sequence of a mature polypeptide having superoxide dismutase (SOD) activity available from Diaporthe nobilis.

SEQ ID NO 43 is the amino acid sequence of a mature polypeptide having superoxide dismutase (SOD) activity available from Tolypocladium sp. XZ2627.

SEQ ID NO 44 is the amino acid sequence of a mature polypeptide having superoxide dismutase (SOD) activity available from Aspergillus japonicus.

SEQ ID NO 45 is the amino acid sequence of a mature polypeptide having superoxide dismutase (SOD) activity available from Metarhizium sp. XZ2431.

SEQ ID NO 46 is the amino acid sequence of a mature polypeptide having superoxide dismutase (SOD) activity available from Armillaria ostoyae.

SEQ ID NO 47 is the amino acid sequence of a mature polypeptide having superoxide dismutase (SOD) activity available from Trichoderma spirale.

SEQ ID NO 48 is the amino acid sequence of a mature polypeptide having superoxide dismutase (SOD) activity available from Aspergillus elegans.

SEQ ID NO 49 is a full-length amino acid sequence from the cDNA sequence of SEQ ID NO 90 from Westerdykella sp. AS85-2. The signal start is amino acid residue 1 and the signal stop is amino acid residue 18.

SEQ ID NO 50 is a full-length amino acid sequence from the cDNA sequence of SEQ ID NO 91 from Aspergillus sp. XZ2669. The signal start is amino acid residue 1 and the signal stop is amino acid residue 19.

SEQ ID NO 51 is a full-length amino acid sequence from the cDNA sequence of SEQ ID NO 92 from Preussia terricola. The signal start is amino acid residue 1 and the signal stop is amino acid residue 19.

SEQ ID NO 52 is a full-length amino acid sequence from the is the cDNA sequence of SEQ ID NO 93 from Kionochaeta sp. The signal start is amino acid residue 1 and the signal stop is amino acid residue 18.

SEQ ID NO 53 is a full-length amino acid sequence from the cDNA sequence of SEQ ID NO 94 from Kionochaeta sp. The signal start is amino acid residue 1 and the signal stop is amino acid residue 18.

SEQ ID NO 54 is a full-length amino acid sequence from the cDNA sequence of SEQ ID NO 95 from Metapochonia bulbillosa. The signal start is amino acid residue 1 and the signal stop is amino acid residue 17.

SEQ ID NO 55 is a full-length amino acid sequence from the cDNA sequence of SEQ ID NO 96 from Xylomelasma sp. XZ0718. The signal start is amino acid residue 1 and the signal stop is amino acid residue 18.

SEQ ID NO 56 is a full-length amino acid sequence from the cDNA sequence of SEQ ID NO 97 from Preussia flanaganii. The signal start is amino acid residue 1 and the signal stop is amino acid residue 25.

SEQ ID NO 57 is a full-length amino acid sequence from the cDNA sequence of SEQ ID NO 99 from Westerdykella sp-46156. The signal start is amino acid residue 1 and the signal stop is amino acid residue 18.

SEQ ID NO 58 is a full-length amino acid sequence from the cDNA sequence of SEQ ID NO 100 from Trichoderma hamatum. The signal start is amino acid residue 1 and the signal stop is amino acid residue 20.

SEQ ID NO 59 is a full-length amino acid sequence from the cDNA sequence of SEQ ID NO 101 from Mycothermus thermophilus.

SEQ ID NO 60 is a full-length amino acid sequence from the cDNA sequence of SEQ ID NO 102 from Cephalotrichiella penicillate. The signal start is amino acid residue 1 and the signal stop is amino acid residue 33.

SEQ ID NO 61 is a full-length amino acid sequence from the cDNA sequence of SEQ ID NO 127 from Chaetomium megalocarpum. The signal start is amino acid residue 1 and the signal stop is amino acid residue 37.

SEQ ID NO 62 is a full-length amino acid sequence from the cDNA sequence of SEQ ID NO 128 from Chaetomium thermophilum var. thermophilum. The signal start is amino acid residue 1 and the signal stop is amino acid residue 33.

SEQ ID NO 63 is a full-length amino acid sequence from the cDNA sequence of SEQ ID NO 129 from Humicola hyalothermophila. The signal start is amino acid residue 1 and the signal stop is amino acid residue 34.

SEQ ID NO 64 is a full-length amino acid sequence from the cDNA sequence of SEQ ID NO 130 from Subramaniula anamorphosa. The signal start is amino acid residue 1 and the signal stop is amino acid residue 36 (based signal alignment with other four Fe-SOD).

SEQ ID NO 65 is a full-length amino acid sequence from the cDNA sequence of SEQ ID NO 131 from Sphingobacterium sp. T2. The signal start is residue 1 and the signal stop is residue 24.

SEQ ID NO 66 is a full-length amino acid sequence from the cDNA sequence of SEQ ID NO 132 from Trichoderma rossicum. The signal start is amino acid residue 1 and the signal stop is amino acid residue 24.

SEQ ID NO 67 is a full-length amino acid sequence from the cDNA sequence of SEQ ID NO 133 from Trichoderma lixii. The signal start is amino acid residue 1 and the signal stop is amino acid residue 19.

SEQ ID NO 68 is a full-length amino acid sequence from the cDNA sequence of SEQ ID NO 134 from Trichoderma sp-54723 The signal start is amino acid residue 1 and the signal stop is amino acid residue 20.

SEQ ID NO 69 is a full-length amino acid sequence from the cDNA sequence of SEQ ID NO 135 from Aspergillus niveus. The signal start is amino acid residue 1 and the signal stop is amino acid residue 16.

SEQ ID NO 70 is a full-length amino acid sequence from the cDNA sequence of SEQ ID NO 136 from Aspergillus templicola. The signal start is amino acid residue 1 and the signal stop is amino acid residue 19.

SEQ ID NO 71 is a full-length amino acid sequence from the cDNA sequence of SEQ ID NO 137 from Pochonia chlamydosporia var. spinulospora. The signal start is amino acid residue 1 and the signal stop is amino acid residue 17.

SEQ ID NO 72 is a full-length amino acid sequence from the cDNA sequence of SEQ ID NO 140 from Trichoderma sp-54723. The signal start is amino acid residue 1 and the signal stop is amino acid residue 20.

SEQ ID NO 73 is a full-length amino acid sequence from the cDNA sequence of SEQ ID NO 141 from Trichoderma sp-44174 The signal start is amino acid residue 1 and the signal stop is amino acid residue 19.

SEQ ID NO 74 is a full-length amino acid sequence from the cDNA sequence of SEQ ID NO 142 from Metapochonia suchlasporia. The signal start is amino acid residue 1 and the signal stop is amino acid residue 17.

SEQ ID NO 75 is a full-length amino acid sequence from the cDNA sequence of SEQ ID NO 143 from Metarhizium marquandii. The signal start is amino acid residue 1 and the signal stop is amino acid residue 17.

SEQ ID NO 76 is a full-length amino acid sequence from the cDNA sequence of SEQ ID NO 144 from Diaporthe nobilis. The signal start is amino acid residue 1 and the signal stop is amino acid residue 17.

SEQ ID NO 77 is a full-length amino acid sequence from the cDNA sequence of SEQ ID NO 145 from Tolypocladium sp. XZ2627. The signal start is amino acid residue 1 and the signal stop is amino acid residue 20.

SEQ ID NO 78 is a full-length amino acid sequence from the cDNA sequence of SEQ ID NO 146 from Aspergillus japonicus The signal start is amino acid residue 1 and the signal stop is amino acid residue 21.

SEQ ID NO 79 is a full-length amino acid sequence from the cDNA sequence of SEQ ID NO 147 from Metarhizium sp. XZ2431. The signal start is amino acid residue 1 and the signal stop is amino acid residue 16.

SEQ ID NO 80 is a full-length amino acid sequence from the cDNA sequence of SEQ ID NO 148 from Armillaria ostoyae. The signal start is amino acid residue 1 and the signal stop is amino acid residue 19.

SEQ ID NO 81 is a full-length amino acid sequence from the cDNA sequence of SEQ ID NO 149 from Trichoderma spirale. The signal start is amino acid residue 1 and the signal stop is amino acid residue 19

SEQ ID NO 82 is a full-length amino acid sequence from the cDNA sequence of SEQ ID NO 150 from Aspergillus elegans. The signal start is amino acid residue 1 and the signal stop is amino acid residue 22.

SEQ ID NO 83 is a full-length amino acid sequence from the cDNA sequence of SEQ ID NO 5 from Trichoderma reesei. The signal start is amino acid residue 1 and the signal stop is amino acid residue 20.

SEQ ID NO 84 is a full-length amino acid sequence from the cDNA sequence of SEQ ID NO 87 from Aspergillus versicolor. The signal start is amino acid residue 1 and the signal stop is amino acid residue 20.

SEQ ID NO 85 is a full-length amino acid sequence from the cDNA sequence of SEQ ID NO 88 from Aspergillus deflectus. The signal start is amino acid residue 1 and the signal stop is amino acid residue 16.

SEQ ID NO 86 is a full-length amino acid sequence from the cDNA sequence of SEQ ID NO 89 from Aspergillus egyptiacus. The signal start is amino acid residue 1 and the signal stop is amino acid residue 19.

SEQ ID NO 87 is a polypeptide coding sequence from Aspergillus versicolor of the polypeptide of SEQ ID NO 84.

SEQ ID NO 88 is a polypeptide coding sequence from Aspergillus deflectus of the polypeptide of SEQ ID NO 85.

SEQ ID NO 89 is a polypeptide coding sequence from Aspergillus egyptiacus of the polypeptide of SEQ ID NO 86.

SEQ ID NO 90 is a polypeptide coding sequence from Westerdykella sp. AS85-2 of the polypeptide of SEQ ID NO: 49.

SEQ ID NO 91 is a polypeptide coding sequence from Aspergillus sp. XZ2669 of the polypeptide of SEQ ID NO: 50.

SEQ ID NO 92 is a polypeptide coding sequence from Preussia terricola of the polypeptide of

SEQ ID NO 51.

SEQ ID NO 93 is a polypeptide coding sequence from Kionochaeta sp. of the polypeptide of SEQ ID NO: 52.

SEQ ID NO 94 is a polypeptide coding sequence from Kionochaeta sp. of the polypeptide of SEQ ID NO 53.

SEQ ID NO 95 is a polypeptide coding sequence from Metapochonia bulbillosa of the polypeptide of SEQ ID NO 54.

SEQ ID NO 96 is a polypeptide coding sequence from Xylomelasma sp. XZ0718 of the polypeptide of SEQ ID NO: 55.

SEQ ID NO 97 is a polypeptide coding sequence from Preussia flanaganii of the polypeptide of

SEQ ID NO: 56.

SEQ ID NO 98 is a polypeptide coding sequence from Cladobotryum sp. of the polypeptide of SEQ ID NO: 18.

SEQ ID NO 99 is a polypeptide coding sequence from Westerdykella sp-46156 of the polypeptide of SEQ ID NO: 57.

SEQ ID NO 100 is a polypeptide coding sequence from Trichoderma hamatum of the polypeptide of SEQ ID NO: 58.

SEQ ID NO 101 is a polypeptide coding sequence from Mycothermus thermophilus of the SEQ ID NO 102 is a polypeptide coding sequence from Cephalotrichiella penicillate of the polypeptide of SEQ ID NO: 160.

SEQ ID NO 103 is the amino acid sequence of a mature polypeptide having catalase activity available from Thermoascus aurantiacus.

SEQ ID NO 104 is the amino acid sequence of a mature polypeptide having catalase activity available from Aspergillus lentulus.

SEQ ID NO 105 is the amino acid sequence of a mature polypeptide having catalase activity available from Talaromyces stipitatus.

SEQ ID NO 106 is the amino acid sequence of a mature polypeptide having catalase activity available from Malbranchea cinnamomea.

SEQ ID NO 107 is the amino acid sequence of a mature polypeptide having catalase activity available from Crassicarpon thermophilum.

SEQ ID NO 108 is the amino acid sequence of a mature polypeptide having catalase activity available from Penicillium emersonii.

SEQ ID NO 109 is the amino acid sequence of a mature polypeptide having catalase activity available from Aspergillus versicolor.

SEQ ID NO 110 is the amino acid sequence of a mature polypeptide having catalase activity available from Thermomucor indicae-seudaticae.

SEQ ID NO 111 is the amino acid sequence of a mature polypeptide having activity available from Aspergillus fumigatus.

SEQ ID NO 112 is the amino acid sequence of a mature polypeptide having catalase activity available from Thermothelomyces thermophilus.

SEQ ID NO 113 is the amino acid sequence of a mature polypeptide having catalase activity available from Curvularia verruculosa.

SEQ ID NO 114 is the amino acid sequence of a mature polypeptide having catalase activity available from Mycothermus thermophilus

SEQ ID NO 115 is the amino acid sequence of a mature polypeptide having catalase activity available from Mycothermus thermophilus.

SEQ ID NO 116 is the amino acid sequence of a mature polypeptide having catalase activity available from Penicillium oxalicum.

SEQ ID NO 117 is the amino acid sequence of a mature polypeptide having catalase activity available from Humicola hyalothermophila.

SEQ ID NO 118 is the amino acid sequence of a mature polypeptide having catalase activity available from Thermoascus crustaceus.

SEQ ID NO 119 is the amino acid sequence of a mature polypeptide having catalase activity available from Thielavia australiensis.

SEQ ID NO 120 is the amino acid sequence of a mature polypeptide having catalase activity available from Thielavia hyrcaniae.

SEQ ID NO 121 is the amino acid sequence of a mature polypeptide having catalase activity available from Neurospora crassa.

SEQ ID NO 122 is the amino acid sequence of a mature polypeptide having catalase activity available from Thermoascus aurantiacus.

SEQ ID NO 123 is the amino acid sequence of a mature polypeptide having catalase activity available from Neurospora crassa.

SEQ ID NO 124 is the amino acid sequence of a mature polypeptide having catalase activity available from Thermoascus aurantiacus.

SEQ ID NO 125 is the amino acid sequence of a mature polypeptide having catalase activity available from Thermoascus aurantiacus.

SEQ ID NO 126 is the amino acid sequence of a mature polypeptide having catalase activity available from Thermoascus aurantiacus.

SEQ ID NO 127 is a polypeptide coding sequence from Chaetomium megalocarpum of the polypeptide of SEQ ID NO 23.

SEQ ID NO 128 is a polypeptide coding sequence from Chaetomium thermophilum var. thermophilum of the polypeptide of SEQ ID NO: 24.

SEQ ID NO 129 is a polypeptide coding sequence from Humicola hyalothermophila of the polypeptide of SEQ ID NO: 25.

SEQ ID NO 130 is a polypeptide coding sequence from Subramaniula anamorphosa of the polypeptide of SEQ ID NO: 26.

SEQ ID NO 131 is a polypeptide coding sequence from Sphingobacterium sp. T2 of the polypeptide of SEQ ID NO 27.

SEQ ID NO 132 is a polypeptide coding sequence from Trichoderma rossicum of the polypeptide of SEQ ID NO 28.

SEQ ID NO 133 is a polypeptide coding sequence from Trichoderma lixii of the polypeptide of SEQ ID NO 29.

SEQ ID NO 134 is a polypeptide coding sequence from Trichoderma sp-54723 of the polypeptide of SEQ ID NO 30.

SEQ ID NO 135 is a polypeptide coding sequence from Aspergillus niveus of the polypeptide of

SEQ ID NO 31.

SEQ ID NO 136 is a polypeptide coding sequence from Aspergillus templicola of the polypeptide of SEQ ID NO 32.

SEQ ID NO 137 is a polypeptide coding sequence from Pochonia chlamydosporia var. spinulospora of the polypeptide of SEQ ID NO 33.

SEQ ID NO 138 is a polypeptide coding sequence from Trichoderma sp-44174 of the polypeptide of SEQ ID NO 34.

SEQ ID NO 139 is a polypeptide coding sequence from Trichoderma rossicum of the polypeptide of SEQ ID NO 36.

SEQ ID NO 140 is a polypeptide coding sequence from Trichoderma sp-54723 of the polypeptide of SEQ ID NO: 38.

SEQ ID NO 141 is a polypeptide coding sequence from Trichoderma sp-44174 of the polypeptide of SEQ ID NO: 39.

SEQ ID NO 142 is a polypeptide coding sequence from Metapochonia suchlasporia of the polypeptide of SEQ ID NO: 40.

SEQ ID NO 143 is a polypeptide coding sequence from Metarhizium marquandii of the polypeptide of SEQ ID NO 41.

SEQ ID NO 144 is a polypeptide coding sequence from Diaporthe nobilis of the polypeptide of

SEQ ID NO 42.

SEQ ID NO 145 is a polypeptide coding sequence from Tolypocladium sp. XZ2627 of the polypeptide of SEQ ID NO 43.

SEQ ID NO 146 is a polypeptide coding sequence from Tolypocladium sp. XZ2627 of the polypeptide of SEQ ID NO 44.

SEQ ID NO 147 is a polypeptide coding sequence from Metarhizium sp. XZ2431 of the polypeptide of SEQ ID NO 45.

SEQ ID NO 148 is a polypeptide coding sequence from Armillaria ostoyae of the polypeptide of SEQ ID NO 46.

SEQ ID NO 149 is a polypeptide coding sequence from Trichoderma spirale of the polypeptide of SEQ ID NO 47.

SEQ ID NO 150 is a polypeptide coding sequence from Aspergillus elegans of the polypeptide of SEQ ID NO 48.

SEQ ID NO 151 is a full-length amino acid sequence of a polypeptide with catalase activity from the cDNA sequence of SEQ ID NO 175 from Thermoascus aurantiacus.

SEQ ID NO 152 is a full-length amino acid sequence of a polypeptide with catalase activity from the cDNA sequence of SEQ ID 176 from Aspergillus lentulus.

SEQ ID NO 153 is a full-length amino acid sequence of a polypeptide with catalase activity from the cDNA sequence of SEQ ID 177 from Talaromyces stipitatus

SEQ ID NO 154 is a full-length amino acid sequence of a polypeptide with catalase activity from the cDNA sequence of SEQ ID 178 from Malbranchea cinnamomea.

SEQ ID NO 155 is a full-length amino acid sequence of a polypeptide with catalase activity from the cDNA sequence of SEQ ID 179 Crassicarpon thermophilum.

SEQ ID NO 156 is a full-length amino acid sequence of a polypeptide with catalase activity from the cDNA sequence of SEQ ID 180 from Penicillium emersonii.

SEQ ID NO 157 is a full-length amino acid sequence of a polypeptide with catalase activity from the cDNA sequence of SEQ ID 181 from Aspergillus versicolor.

SEQ ID NO 158 is a full-length amino acid sequence of a polypeptide with catalase activity from the cDNA sequence of SEQ ID 182 from Thermomucor indicae-seudaticae

SEQ ID NO 159 is a full-length amino acid sequence of a polypeptide with catalase activity from the cDNA sequence of SEQ ID 183 from Aspergillus fumigatus

SEQ ID NO 160 is a full-length amino acid sequence of a polypeptide with catalase activity from the cDNA sequence of SEQ ID 184 from Thermothelomyces thermophilus.

SEQ ID NO 161 is a full-length amino acid sequence of a polypeptide with catalase activity from the cDNA sequence of SEQ ID 185 from Curvularia verruculosa.

SEQ ID NO 162 is a full-length amino acid sequence of a polypeptide with catalase activity from the cDNA sequence of SEQ ID 186 from Mycothermus thermophilus.

SEQ ID NO 163 is a full-length amino acid sequence with catalase activity from the cDNA sequence of SEQ ID 187 from Mycothermus thermophilus.

SEQ ID NO 164 is a full-length amino acid sequence with catalase activity from the cDNA sequence of SEQ ID 188 from Penicillium oxalicum

SEQ ID NO 165 is a full-length amino acid sequence with catalase activity from the cDNA sequence of SEQ ID 189 from Humicola hyalothermophila.

SEQ ID NO 166 is a full-length amino acid sequence with catalase activity from the cDNA sequence of SEQ ID 190 from Thermoascus crustaceus.

SEQ ID NO 167 is a full-length amino acid sequence with catalase activity from the cDNA sequence of SEQ ID 191 from Thielavia australiensis.

SEQ ID NO 168 is a full-length amino acid sequence with catalase activity from the cDNA sequence of SEQ ID 192 from Thielavia hyrcaniae.

SEQ ID NO 169 is a full-length amino acid sequence with catalase activity from the cDNA sequence of SEQ ID 193 from Neurospora crassa.

SEQ ID NO 170 is a full-length amino acid sequence with catalase activity from the cDNA sequence of SEQ ID 194 from Thermoascus aurantiacus.

SEQ ID NO 171 is a full-length amino acid sequence with catalase activity from the cDNA sequence of SEQ ID 195 from Neurospora crassa.

SEQ ID NO 172 is a full-length amino acid sequence with catalase activity from the cDNA sequence of SEQ ID 196 from Thermoascus aurantiacus.

SEQ ID NO 173 is a full-length amino acid sequence with catalase activity from the cDNA sequence of SEQ ID 197 from Thermoascus aurantiacus.

SEQ ID NO 174 is a full-length amino acid sequence with catalase activity from the cDNA sequence of SEQ ID 198 from Thermoascus aurantiacus.

SEQ ID NO 175 is a polypeptide coding sequence from Thermoascus aurantiacus coding for the polypeptide of SEQ ID NO 151.

SEQ ID NO 176 is a polypeptide coding sequence from Aspergillus lentulus coding for the polypeptide of SEQ ID NO 152.

SEQ ID NO 177 is a polypeptide coding sequence from Talaromyces stipitatus coding for the polypeptide of SEQ ID NO 153.

SEQ ID NO 178 is a polypeptide coding sequence from Malbranchea cinnamomea coding for the polypeptide of SEQ ID NO 154.

SEQ ID NO 179 is a polypeptide coding sequence from Crassicarpon thermophilum coding for the polypeptide of SEQ ID NO 155.

SEQ ID NO 180 is a polypeptide coding sequence from Penicillium emersonii coding for the polypeptide of SEQ ID NO 156.

SEQ ID NO 181 is a polypeptide coding sequence from Aspergillus versicolor coding for the polypeptide of SEQ ID NO 157.

SEQ ID NO 182 is a polypeptide coding sequence from Thermomucor indicae-seudaticae coding for the polypeptide of SEQ ID NO 158.

SEQ ID NO 183 is a polypeptide coding sequence from Aspergillus fumigatus coding for the polypeptide of SEQ ID NO 159.

SEQ ID NO 184 is a polypeptide coding sequence from Thermothelomyces thermophilus coding for the polypeptide of SEQ ID NO 160.

SEQ ID NO 185 is a polypeptide coding sequence from Curvularia verruculosa coding for the polypeptide of SEQ ID NO 161.

SEQ ID NO 186 is a polypeptide coding sequence from Mycothermus thermophilus coding for the polypeptide of SEQ ID NO 162.

SEQ ID NO 187 is a polypeptide coding sequence from Mycothermus thermophilus coding for the polypeptide of SEQ ID NO 163.

SEQ ID NO 188 is a polypeptide coding sequence from Penicillium oxalicum coding for the polypeptide of SEQ ID NO 164.

SEQ ID NO 189 is a polypeptide coding sequence from Humicola hyalothermophila coding for the polypeptide of SEQ ID NO 165.

SEQ ID NO 190 is a polypeptide coding sequence from Thermoascus crustaceus coding for the polypeptide of SEQ ID NO 160.

SEQ ID NO 191 is a polypeptide coding sequence from Thielavia australiensis coding for the polypeptide of SEQ ID NO 167.

SEQ ID NO 192 is a polypeptide coding sequence from Thielavia hyrcaniae coding for the polypeptide of SEQ ID NO 168.

SEQ ID NO 193 is a polypeptide coding sequence from Neurospora crassa coding for the polypeptide of SEQ ID NO 169.

SEQ ID NO 194 is a polypeptide coding sequence from Thermoascus aurantiacus coding for the polypeptide of SEQ ID NO 170.

SEQ ID NO 195 is a polypeptide coding sequence from Neurospora crassa coding for the polypeptide of SEQ ID NO 171.

SEQ ID NO 196 is a polypeptide coding sequence from Thermoascus aurantiacus coding for the polypeptide of SEQ ID NO 172.

SEQ ID NO 197 is a polypeptide coding sequence from Thermoascus aurantiacus coding for the polypeptide of SEQ ID NO 173.

SEQ ID NO 198 is a polypeptide coding sequence from Thermoascus aurantiacus coding for the polypeptide of SEQ ID NO 174.

SEQ ID NO 199 is the amino acid sequence of a mature polypeptide having superoxide dismutase (SOD) activity available from Trichoderma sinuosum (O434SD).

SEQ ID NO 200 is the amino acid sequence of a mature polypeptide having superoxide dismutase (SOD) activity available from Trichoderma virens (O32VB5).

SEQ ID NO 201 is the amino acid sequence of a mature polypeptide having superoxide dismutase (SOD) activity available from Trichoderma harzianum (O4).

SEQ ID NO 202 is the amino acid sequence of a mature polypeptide having superoxide dismutase (SOD) activity available from Fusicolla acetilerea.

SEQ ID NO 203 is the amino acid sequence of a mature polypeptide having superoxide dismutase (SOD) activity available from Plectosphaerella sp. 1-29.

SEQ ID NO 204 is the amino acid sequence of a mature polypeptide having superoxide dismutase (SOD) activity available from Mariannaea punicea.

SEQ ID NO 205 is the amino acid sequence of a mature polypeptide having superoxide dismutase (SOD) activity available from Penicillium oxalicum.

SEQ ID NO 206 is the amino acid sequence of a mature polypeptide having superoxide dismutase (SOD) activity available from Colletotrichum sp-71086.

SEQ ID NO 207 is the amino acid sequence of a mature polypeptide having superoxide dismutase (SOD) activity available from Aspergillus sp. nov. XZ3202.

SEQ ID NO 208 is the amino acid sequence of a mature polypeptide having superoxide dismutase (SOD) activity available from Trichoderma parapiluliferum.

SEQ ID NO 209 is the amino acid sequence of a mature polypeptide having superoxide dismutase (SOD) activity available from Aspergillus sp. nov. XZ3202.

SEQ ID NO 210 is a full-length amino acid sequence from the cDNA sequence of SEQ ID NO 221 from Trichoderma sinuosum. The signal start is amino acid residue 1 and the signal stop is amino acid residue 19.

SEQ ID NO 211 is a full-length amino acid sequence from the cDNA sequence of SEQ ID NO 222 from Trichoderma virens. The signal start is amino acid residue 1 and the signal stop is amino acid residue 19.

SEQ ID NO 212 is a full-length amino acid sequence from the cDNA sequence of SEQ ID NO 223 from Trichoderma harzianum. The signal start is amino acid residue 1 and the signal stop is amino acid residue 19.

SEQ ID NO 213 is a full-length amino acid sequence from the cDNA sequence of SEQ ID NO 224 from Fusicolla acetilerea. The signal start is amino acid residue 1 and the signal stop is amino acid residue 19. With no native signal, a signal and propeptide was added at its N-terminal, and MVK was removed from the N-terminal.

SEQ ID NO 214 is a full-length amino acid sequence from the cDNA sequence of SEQ ID NO 225 from Plectosphaerella sp. 1-29.

SEQ ID NO 215 is a full-length amino acid sequence from the cDNA sequence of SEQ ID NO 226 from Mariannaea punicea.

SEQ ID NO 216 is a full-length amino acid sequence from the cDNA sequence of SEQ ID NO 227 from Penicillium oxalicum.

SEQ ID NO 217 is a full-length amino acid sequence from the cDNA sequence of SEQ ID NO 228 from Colletotrichum sp-71086. The signal start is amino acid residue 1 and the signal stop is amino acid residue 20.

SEQ ID NO 218 is a full-length amino acid sequence from the cDNA sequence of SEQ ID NO 229 from Aspergillus sp. nov. XZ3202. The signal start is amino acid residue 1 and the signal stop is amino acid residue 16.

SEQ ID NO 219 is a full-length amino acid sequence from the cDNA sequence of SEQ ID NO 230 from Trichoderma parapiluliferum. The signal start is amino acid residue 1 and the signal stop is amino acid residue 18.

SEQ ID NO 220 is a full-length amino acid sequence from the cDNA sequence of SEQ ID NO 231 from Aspergillus sp. nov. XZ3202. The signal start is amino acid residue 1 and the signal stop is amino acid residue 19.

SEQ ID NO 221 is a polypeptide coding sequence of the polypeptide of SEQ ID NO 210 from Trichoderma sinuosum.

SEQ ID NO 222 is a polypeptide coding sequence of the polypeptide of SEQ ID NO 211 from Trichoderma virens.

SEQ ID NO 223 is a polypeptide coding sequence of the polypeptide of SEQ ID NO 212 from Trichoderma harzianum.

SEQ ID NO 224 is a polypeptide coding sequence of the polypeptide of SEQ ID NO 213 from Fusicolla acetilerea.

SEQ ID NO 225 is a polypeptide coding sequence of the polypeptide of SEQ ID NO 214 from Plectosphaerella sp. 1-29.

SEQ ID NO 226 is a polypeptide coding sequence of the polypeptide of SEQ ID NO 215 from Mariannaea punicea.

SEQ ID NO 227 is a polypeptide coding sequence of the polypeptide of SEQ ID NO 216 from Penicillium oxalicum.

SEQ ID NO 228 is a polypeptide coding sequence of the polypeptide of SEQ ID NO 217 from Colletotrichum sp-71086.

SEQ ID NO 229 is a polypeptide coding sequence of the polypeptide of SEQ ID NO: 218 from Aspergillus sp. nov. XZ3202 (O14ETD).

SEQ ID NO 230 is a polypeptide coding sequence of the polypeptide of SEQ ID NO: 219 from Trichoderma parapiluliferum (O72ZVS).

SEQ ID NO 231 is a polypeptide coding sequence of the polypeptide of SEQ ID NO: 220 from Aspergillus sp. nov. XZ3202.

SEQ ID NO 232 is the amino acid sequence of a mature polypeptide having superoxide dismutase (SOD) activity from Mucor sp. XZ2651.

SEQ ID NO 233 is the amino acid sequence of a mature polypeptide having superoxide dismutase (SOD) activity from Rhizomucor miehei.

SEQ ID NO 234 is the amino acid sequence of a mature polypeptide having superoxide dismutase (SOD) activity from Mucor sp. XZ2651.

SEQ ID NO 235 is the amino acid sequence of a mature polypeptide having superoxide dismutase (SOD) activity from Amphisphaeriaceae sp-43674.

SEQ ID NO 236 is the amino acid sequence of a mature polypeptide having superoxide dismutase (SOD) activity from Humicola fuscoatra.

SEQ ID NO 237 is the amino acid sequence of a mature polypeptide having superoxide dismutase (SOD) activity from Valsaria rubricosa.

SEQ ID NO 238 is a full-length amino acid sequence from the cDNA sequence of SEQ ID NO 244 DNA from Mucor sp. XZ2651. The signal start is amino acid residue 1 and the signal stop is amino acid residue 17.

SEQ ID NO 239 is a full-length amino acid sequence from the cDNA sequence of SEQ ID NO 245 DNA from Rhizomucor miehei. The signal start is amino acid residue 1 and the signal stop is amino acid residue 13.

SEQ ID NO 240 is a full-length amino acid sequence from the cDNA sequence of SEQ ID NO 246 DNA from Mucor sp. XZ2651. The signal start is amino acid residue 1 and the signal stop is amino acid residue 17.

SEQ ID NO 241 is a full-length amino acid sequence from the cDNA sequence of SEQ ID NO 247 DNA from Amphisphaeriaceae sp-43674. The signal start is amino acid residue 1 and the signal stop is amino acid residue 18.

SEQ ID NO 242 is a full-length amino acid sequence from the cDNA sequence of SEQ ID NO 248 DNA from Humicola fuscoatra. The signal start is amino acid residue 1 and the signal stop is amino acid residue 17.

SEQ ID NO 243 is a full-length amino acid sequence from the cDNA sequence of SEQ ID NO 249 DNA from Valsaria rubricosa. The signal start is amino acid residue 1 and the signal stop is amino acid residue 18.

SEQ ID NO 244 is a polypeptide coding sequence of the polypeptide of SEQ ID NO 238 from Mucor sp. XZ2651.

SEQ ID NO 245 is a polypeptide coding sequence of the polypeptide of SEQ ID NO 239 from Rhizomucor miehei

SEQ ID NO 246 is a polypeptide coding sequence of the polypeptide of SEQ ID NO 240 from Mucor sp. XZ2651.

SEQ ID NO 247 is a polypeptide coding sequence of the polypeptide of SEQ ID NO 241 from Amphisphaeriaceae sp-43674.

SEQ ID NO 248 is a polypeptide coding sequence of the polypeptide of SEQ ID NO 242 from from Humicola fuscoatra.

SEQ ID NO 249 is a polypeptide coding sequence of the polypeptide of SEQ ID NO 243 from Valsaria rubricosa.

SEQ ID NO 250 is the amino acid sequence of a mature polypeptide having catalase activity from Thermoascus aurantiacus comprising 724 amino acid residues. SEQ ID NO 250 the mature polypeptide of the catalase sold under the tradename Terminox™.

SEQ ID NO 251 is a full-length amino acid sequence of a polypeptide from Thermoascus aurantiacus having catalase activity comprising 740 amino acid residues, sold under the tradename Terminox™. The signal start is amino acid residue 1 and the signal stop is amino acid residue 16.

TABLE 1a
Super Oxide Dismutases and their polypeptide coding sequences

| | SEQ ID NO | SEQ ID NO | SEQ ID NO |
|---|---|---|---|
| Source | Mature peptide | Full polypeptide | DNA |
| Trichoderma reesei | SED ID NO 1 | SEQ ID NO 83 | SEQ ID NO 5 |
| Aspergillus versicolor | SED ID NO 2 | SEQ ID NO 84 | SEQ ID NO 87 |
| Aspergillus deflectus | SEQ ID NO 3 | SEQ ID NO 85 | SEQ ID NO 88 |
| Aspergillus egyptiacus | SEQ ID NO 4 | SEQ ID NO 86 | SEQ ID NO 89 |
| Westerdykella sp. AS85-2 | SEQ ID NO 9 | SEQ ID NO 49 | SEQ ID NO 90 |
| Aspergillus sp. XZ2669 | SEQ ID NO 10 | SEQ ID NO 50 | SEQ ID NO 91 |
| Preussia terricola | SEQ ID NO 11 | SEQ ID NO 51 | SEQ ID NO 92 |
| Kionochaeta sp. | SEQ ID NO 12 | SEQ ID NO 52 | SEQ ID NO 93 |
| Kionochaeta sp. | SEQ ID NO 13 | SEQ ID NO 53 | SEQ ID NO 94 |
| Metapochonia bulbillosa | SEQ ID NO 14 | SEQ ID NO 54 | SEQ ID NO 95 |
| Xylomelasma sp. XZ0718 | SEQ ID NO 15 | SEQ ID NO 55 | SEQ ID NO 96 |
| Preussia flanaganii | SEQ ID NO 16 | SEQ ID NO 56 | SEQ ID NO 97 |
| Cladobotryum sp. | SEQ ID NO 17 | SEQ ID NO 18 | SEQ ID NO 98 |
| Westerdykella sp-46156 | SEQ ID NO 19 | SEQ ID NO 57 | SEQ ID NO 99 |
| Trichoderma hamatum | SEQ ID NO 20 | SEQ ID NO 58 | SEQ ID NO 100 |
| Mycothermus thermophilus | SEQ ID NO 21 | SEQ ID NO 59 | SEQ ID NO 101 |
| Cephalotrichiella penicillata | SEQ ID NO 22 | SEQ ID NO 60 | SEQ ID NO 102 |
| Chaetomium megalocarpum | SEQ ID NO 23 | SEQ ID NO 61 | SEQ ID NO 127 |
| Chaetomium thermophilum | SEQ ID NO 24 | SEQ ID NO 62 | SEQ ID NO 128 |
| var. thermophilum | | | |
| Humicola hyalothermophila | SEQ ID NO 25 | SEQ ID NO 63 | SEQ ID NO 129 |
| Subramaniula anamorphosa | SEQ ID NO 26 | SEQ ID NO 64 | SEQ ID NO 130 |
| Sphingobacterium sp. T2 | SEQ ID NO 27 | SEQ ID NO 65 | SEQ ID NO 131 |
| Trichoderma rossicum | SEQ ID NO 28 | SEQ ID NO 66 | SEQ ID NO 132 |
| Trichoderma lixii | SEQ ID NO 29 | SEQ ID NO 67 | SEQ ID NO 133 |
| Trichoderma sp-54723 | SEQ ID NO 30 | SEQ ID NO 68 | SEQ ID NO 134 |
| Aspergillus niveus | SEQ ID NO 31 | SEQ ID NO 69 | SEQ ID NO 135 |
| Aspergillus templicola | SEQ ID NO 32 | SEQ ID NO 70 | SEQ ID NO 136 |
| Pochonia chlamydosporia | SEQ ID NO 33 | SEQ ID NO 71 | SEQ ID NO 137 |
| var. spinulospora | | | |
| Trichoderma sp-44174 | SEQ ID NO 34 | SEQ ID NO 35 | SEQ ID NO 138 |
| Trichoderma rossicum | SEQ ID NO 36 | SEQ ID NO 37 | SEQ ID NO 139 |
| Trichoderma sp-54723 | SEQ ID NO 38 | SEQ ID NO 72 | SEQ ID NO 140 |
| Trichoderma sp-44174 | SEQ ID NO 39 | SEQ ID NO 73 | SEQ ID NO 141 |
| Metapochonia suchlasporia | SEQ ID NO 40 | SEQ ID NO 74 | SEQ ID NO 142 |
| Metarhizium marquandii | SEQ ID NO 41 | SEQ ID NO 75 | SEQ ID NO 143 |
| Diaporthe nobilis | SEQ ID NO 42 | SEQ ID NO 76 | SEQ ID NO 144 |
| Tolypocladium sp. XZ2627 | SEQ ID NO 43 | SEQ ID NO 77 | SEQ ID NO 145 |
| Aspergillus japonicus | SEQ ID NO 44 | SEQ ID NO 78 | SEQ ID NO 146 |
| Metarhizium sp. XZ2431 | SEQ ID NO 45 | SEQ ID NO 79 | SEQ ID NO 147 |
| Armillaria ostoyae | SEQ ID NO 46 | SEQ ID NO 80 | SEQ ID NO 148 |
| Trichoderma spirale | SEQ ID NO 47 | SEQ ID NO 81 | SEQ ID NO 149 |
| Aspergillus elegans | SEQ ID NO 48 | SEQ ID NO 82 | SEQ ID NO 150 |
| Trichoderma sinuosum | SEQ ID NO 199 | SEQ ID NO 210 | SEQ ID NO 221 |
| Trichoderma virens | SEQ ID NO 200 | SEQ ID NO 211 | SEQ ID NO 222 |
| Trichoderma harzianum | SEQ ID NO 201 | SEQ ID NO 212 | SEQ ID NO 223 |
| Fusicolla acetilerea | SEQ ID NO 202 | SEQ ID NO 213 | SEQ ID NO 224 |
| Plectosphaerella sp. 1-29 | SEQ ID NO 203 | SEQ ID NO 214 | SEQ ID NO 225 |
| Mariannaea punicea | SEQ ID NO 204 | SEQ ID NO 215 | SEQ ID NO 226 |
| Penicillium oxalicum | SEQ ID NO 205 | SEQ ID NO 216 | SEQ ID NO 227 |
| Colletotrichum sp-71086 | SEQ ID NO 206 | SEQ ID NO 217 | SEQ ID NO 228 |
| Aspergillus sp. nov. | SEQ ID NO 207 | SEQ ID NO 218 | SEQ ID NO 229 |
| XZ3202 | | | |
| Trichoderma | SEQ ID NO 208 | SEQ ID NO 219 | SEQ ID NO 230 |
| parapiluliferum | | | |
| Aspergillus sp. nov. | SEQ ID NO 209 | SEQ ID NO 220 | SEQ ID NO 231 |
| XZ3202 | | | |
| Mucor sp. XZ2651 | SEQ ID NO 232 | SEQ ID NO 238 | SEQ ID NO 244 |
| Rhizomucor miehei | SEQ ID NO 233 | SEQ ID NO 239 | SEQ ID NO 245 |
| Mucor sp. XZ2651 | SEQ ID NO 234 | SEQ ID NO 240 | SEQ ID NO 246 |
| Amphisphaeriaceae -sp | SEQ ID NO 235 | SEQ ID NO 241 | SEQ ID NO 247 |
| 43674 | | | |
| Humicola fuscoatra | SEQ ID NO 236 | SEQ ID NO 242 | SEQ ID NO 248 |
| Valsaria rubricosa | SEQ ID NO 237 | SEQ ID NO 243 | SEQ ID NO 249 |

TABLE 1b
Catalases and their polypeptide coding sequences

| | SEQ ID NO | SEQ ID NO | SEQ ID NO |
|---|---|---|---|
| Source | Mature peptide | Full polypeptide | DNA |
| Aspergillus niger | SEQ ID NO 6 | na | na |
| Aspergillus niger | SEQ ID NO 7 | na | SEQ ID NO 8 |
| Thermoascus aurantiacus | SEQ ID NO 103 | SEQ ID NO 151 | SEQ ID NO 175 |
| Aspergillus lentulus | SEQ ID NO 104 | SEQ ID NO 152 | SEQ ID NO 176 |
| Talaromyces stipitatus | SEQ ID NO 105 | SEQ ID NO 153 | SEQ ID NO 177 |
| Malbranchea cinnamomea | SEQ ID NO 106 | SEQ ID NO 154 | SEQ ID NO 178 |
| Crassicarpon thermophilum | SEQ ID NO 107 | SEQ ID NO 155 | SEQ ID NO 179 |
| Penicillium emersonii | SEQ ID NO 108 | SEQ ID NO 156 | SEQ ID NO 180 |
| Aspergillus versicolor | SEQ ID NO 109 | SEQ ID NO 157 | SEQ ID NO 181 |
| Thermomucor indicae- | SEQ ID NO 110 | SEQ ID NO 158 | SEQ ID NO 182 |
| seudaticae | | | |
| Aspergillus fumigatus | SEQ ID NO 111 | SEQ ID NO 159 | SEQ ID NO 183 |
| Thermothelomyces | SEQ ID NO 112 | SEQ ID NO 160 | SEQ ID NO 184 |
| thermophilus | | | |
| Curvularia verruculosa | SEQ ID NO 113 | SEQ ID NO 161 | SEQ ID NO 185 |
| Mycothermus thermophilus | SEQ ID NO 114 | SEQ ID NO 162 | SEQ ID NO 186 |
| Mycothermus thermophilus | SEQ ID NO 115 | SEQ ID NO 163 | SEQ ID NO 187 |
| Penicillium oxalicum | SEQ ID NO 116 | SEQ ID NO 164 | SEQ ID NO 188 |
| Humicola hyalothermophila | SEQ ID NO 117 | SEQ ID NO 165 | SEQ ID NO 189 |
| Thermoascus crustaceus | SEQ ID NO 118 | SEQ ID NO 166 | SEQ ID NO 190 |
| Thielavia australiensis | SEQ ID NO 119 | SEQ ID NO 167 | SEQ ID NO 191 |
| Thielavia hyrcaniae | SEQ ID NO 120 | SEQ ID NO 168 | SEQ ID NO 192 |
| Neurospora crassa | SEQ ID NO 121 | SEQ ID NO 169 | SEQ ID NO 193 |
| Thermoascus aurantiacus | SEQ ID NO 122 | SEQ ID NO 170 | SEQ ID NO 194 |
| Neurospora crassa | SEQ ID NO 123 | SEQ ID NO 171 | SEQ ID NO 195 |
| Thermoascus aurantiacus | SEQ ID NO 124 | SEQ ID NO 172 | SEQ ID NO 196 |
| Thermoascus aurantiacus | SEQ ID NO 125 | SEQ ID NO 173 | SEQ ID NO 197 |
| Thermoascus aurantiacus | SEQ ID NO 126 | SEQ ID NO 174 | SEQ ID NO 198 |
| Thermoascus aurantiacus | SEQ ID NO 250 | SEQ ID NO 251 | |

### DETAILED DESCRIPTION OF THE INVENTION

The invention is related one or more polypeptides having superoxide dismutase activity in animal feed wherein the superoxide dismutase is of fungal origin.

### Definitions

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Feed Conversion Ratio (FCR): FCR is a measure of an animal's efficiency in converting feed mass into increases of the desired output. Animals raised for meat such as swine, poultry and fish the output is the mass gained by the animal. Specifically, FCR is calculated as feed intake divided by weight gain, all over a specified period. Improvement in FCR means reduction of the FCR value. An FCR improvement of 2% means that the FCR was reduced by 2%.

Feed Premix: The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

European Production Efficiency Factor (EPEF): The European Production Efficiency Factor is a way of comparing the performance of animals. This single-figure facilitates comparison of performance within and among farms and can be used to assess environmental, climatic and animal management variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g.

Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has SOD activity.several) amino acids absent from the amino and/or carboxyl terminus of a mature polypeptide or domain; wherein the fragment has SOD activity.

In one aspect, a fragment of a GH24 SOD (such as one of SEQ ID NO: 63 to 71) comprises at least 230 amino acids, such as at least 235 amino acids, at least 240 amino acids, or at least 245 amino acids and has SOD activity. In another aspect, a fragment of a GH24 SOD (such as one of SEQ ID NO: 63 to 71) comprises at least 90% of the length of the mature polypeptide, such as at least 92%, at least 94%, at least 96%, at least 98% or at least 99% of the length of the mature polypeptide and has SOD activity.

In one aspect, a fragment of a GH25 SOD (such as one of SEQ ID NO: 1 to 72) comprises at least 180 amino acids, such as at least 185 amino acids, at least 190 amino acids, at least 195 amino acids, at least 200 amino acids, at least 205 amino acids or at least 210 amino acids and has SOD activity. In another aspect, a fragment of a GH25 SOD (such as one of SEQ ID NO: 1 to 72) comprises at least 90% of the length of the mature polypeptide, such as at least 92%, at least 94%, at least 96%, at least 98% or at least 99% of the length of the mature polypeptide and has SOD activity.

Fungal origin: The term “fungal origin is intended to mean, in reference to a superoxide dismutase, that the source of the enzyme in a fungus. A fungus is any member of the group of eukaryotic organisms that includes microorganisms such as yeasts and molds, as well as the more familiar mushrooms. These organisms are classified as a kingdom, fungi. Currently, seven phyla are proposed: Microsporidia, Chytridiomycota, Blastocladiomycota, Neocallimastigomycota, Glomeromycota, Ascomycota, and Basidiomycota. Suitable examples include, without limitation, Trichoderma reesei, Aspergillus versicolor, Aspergillus deflectus, Aspergillus egyptiacus, Westerdykella sp. AS85-2, Aspergillus sp. XZ2669, Preussia terricola, Kionochaeta sp., Metapochonia bulbillosa, Xylomelasma sp. XZ0718, Preussia flanaganii, Cladobotryum sp., Westerdykella sp-46156, Trichoderma hamatum, Mycothermus thermophilus, Cephalotrichiella penicillate, Chaetomium megalocarpum, Chaetomium thermophilum var. thermophilum, Humicola hyalothermophila, Subramaniula anamorphosa, Sphingobacterium sp. T2, Trichoderma rossicum, Trichoderma lixii, Trichoderma sp-54723, Aspergillus niveus, Aspergillus templicola, Pochonia chlamydosporia var. spinulospora, Trichoderma sp-44174, Trichoderma rossicum, Trichoderma sp-54723, Trichoderma sp-44174, Metapochonia suchlasporia, Metarhizium marquandii, Diaporthe nobilis, Tolypocladium sp. XZ2627, Aspergillus japonicus, Metarhizium sp. XZ2431, Armillaria ostoyae, Trichoderma spirale, Aspergillus elegans, Trichoderma sinuosum, Trichoderma virens, Trichoderma harzianum, Fusicolla acetilerea, Plectosphaerella sp. 1-29, Mariannaea punicea, Penicillium oxalicum, Colletotrichum sp-71086, Aspergillus sp. nov. XZ3202, Trichoderma parapiluliferum, Aspergillus sp. nov. XZ3202, Mucor sp. XZ2651, Rhizomucor miehei, Mucor sp. XZ2651, Amphisphaeriaceae-sp 43674, Humicola fuscoatra and Valsaria rubricosa.

Heat Stress: Heat stress occurs when an animal's heat load is greater than its capacity to lose heat. Pigs andother animals likely experience headaches, irritability and lethargy when they are too hot and have insufficient water. One or more of the following are typically observed with heat stress: increased breathing rate and sweating, increased water intake, decreased feed intake.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Physical determination of the mature N terminus of SODs was done with Mass Spectrometry. Samples were diluted to 0.1 mg/ml in water. If they were to be deglycosylated before analysis, the samples were suspended in 50 mM Ammonium acetate buffer pH 5.5. The samples are then placed in an Ultimate 3000 UHPLC system (Thermo Scientific) at 8 degrees C. and run over an Advance Bio-RP desalting column (Agilent) The solvents used were A: LC/MS grade water with 0.1% formic acid, solvent: B 95% acetonitrile with 0.1% formic acid. The gradient was 5-80% B over 5 minutes. Post column the protein eluent was analyzed in a Bruker Maxis II mass spectrometer (Bremen Germany) and the resulting trace was analyzed by the supplied Bruker data analysis software. The deconvoluted spectrum was then compared to the calculated molecular weight with the expected N and C terminals using GPMAW (General Protein/Mass Analysis for Windows) software version 12.20. If the values match within 1 Dalton, a match was concluded.

Nutritional Stress: In pigs, poultry and other animals, symptoms of nutritional stress include impaired growth, immune suppression, reduced gut health, reduced gut integrity, shift in gut microflora and vomiting. In poultry, further symptoms include decreased egg production, decreased hatchability, gizzaed lesions, increased suspectibility to necrotic enteritis.

Obtained or obtainable from: The term “obtained or obtainable from” means that the polypeptide may be found in an organism from a specific taxonomic rank. In one embodiment, the polypeptide is obtained or obtainable from the kingdom Fungi, wherein the term kingdom is the taxonomic rank. In a preferred embodiment, the polypeptide is obtained or obtainable from the phylum Ascomycota, wherein the term phylum is the taxonomic rank. In another preferred embodiment, the polypeptide is obtained or obtainable from the subphylum Pezizomycotina, wherein the term subphylum is the taxonomic rank. In another preferred embodiment, the polypeptide is obtained or obtainable from the class Eurotiomycetes, wherein the term class is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Oxidative Stress: The term “oxidative stress” is intended to mean an imbalance between oxidants and reductants (antioxidants) at the cellular or individual level. Oxidative damage is one result of such an imbalance and includes oxidative modification of cellular macromolecules, cell death by apoptosis or necrosis, as well as structural tissue damage by means of reactive oxygen and nitrogen species (ROS, RNS).

Roughage: The term “roughage” means dry plant material with high levels of fiber, such as fiber, bran, husks from seeds and grains and crop residues (such as stover, copra, straw, chaff, sugar beet waste).

Secreted Enzyme: A secreted enzyme is an exoenzyme, or extracellular enzyme, in that is an enzyme that is secreted by a cell and functions outside that cell.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Tm: The term Tm, as used in the Examples refers to the temperature at which 50% of the protein molecules are unfolded and 50% of the protein molecules are folded.

Variant: The term “variant” means a polypeptide having SOD activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a SOD variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the SOD activity of the parent SOD, such as SEQ ID NO: 1 to 5.

Nutrient: The term “nutrient” in the present invention means components or elements contained in dietary feed for an animal, including water-soluble ingredients, fat-soluble ingredients and others. The example of water-soluble ingredients includes but is not limited to carbohydrates such as saccharides including glucose, fructose, galactose and starch; minerals such as calcium, magnesium, zinc, phosphorus, potassium, sodium and sulfur; nitrogen source such as amino acids and proteins, vitamins such as vitamin B1, vitamin B2, vitamin B3, vitamin B6, folic acid, vitamin B12, biotin and phatothenic acid. The example of the fat-soluble ingredients includes but is not limited to fats such as fat acids including saturated fatty acids (SFA); mono-unsaturated fatty acids (MUFA) and poly-unsaturated fatty acids (PUFA), fibre, vitamins such as vitamin A, vitamin E and vitamin K.

### Super Oxide Dismutases

The invention is directed at least in part to novel polypeptides having superoxide dismutase activity.

One aspect of the invention is directed to an isolated polypeptide having superoxide dismutase activity selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

One aspect of the invention is directed to an isolated polypeptide having superoxide dismutase activity comprising an amino acid sequence selected from the group consisting of:
a. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1;
b. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2;
c. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3;
d. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4;
e. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9;
f. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10;
g. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11;
h. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 12;
i. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 13;
j. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 14;
k. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 15;
l. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 16;
m. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17;
n. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19;
o. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 20;
p. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21;
q. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22;
r. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23;
s. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 24;
t. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 sequence identity to the amino acid sequence of SEQ ID NO: 25;
u. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26;
v. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 27;
w. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 28;
x. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 29;
y. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 30;
z. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 31;
aa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 32;
bb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33;
cc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 34;
dd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 36;
ee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 38;
ff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 39;
gg. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 42;
jj. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43;
kk. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44;
ll. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 45;
mm. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 46;
nn. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 47; and
oo. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 48;
pp. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:199;
qq. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:200;
rr. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO 201;
ss. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:202;
tt. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:203;
uu. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:204;
vv. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 205;
ww. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:206;
xx. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:207;
yy. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:208;
zz. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:209;
aaa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:232;
bbb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:233;
ccc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:234;
ddd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:235;
eee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:236; and
fff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:237.

On aspect of the invention is directed to an isolated polypeptide having superoxide dismutase activity selected from the group consisting of
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

One aspect of the invention is directed to an isolated polypeptide of having superoxide dismutase activity selected from the group consisting of
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 307 of SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 206 of SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 154 of SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 188 of SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 184 of SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 249 of SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 178 of SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 248 of SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 274 of SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 26 to 257 of SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 261 of SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 269 of SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 151 of SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 228 of SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 37 to 233 of SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 227 of SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 34 to 230 of SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 37 to 233 of SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 25 to 227 of SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 211 of SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 218 of SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 218 of SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 182 of SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 185 of SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 185 of SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 304 of SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 310 of SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 303 of SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 209 of SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 209 of SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 22 to 183 of SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 193 of SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 177 of SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 215 of SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 182 of SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 154 of SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 150 of SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 247 of SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 154 of SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 316 of SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 193 of SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 14 to 164 of SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 184 of SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 242 of SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 261 of SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 258 of SEQ ID NO 243.

One aspect of the invention is directed to an isolated polypeptide of having superoxide dismutase activity selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.

### Sequences of Superoxide Dismutases of the Animal Feed

Superoxide dismutase (SOD, EC 1.15.1.1) is an enzyme that alternately catalyzes the dismutation (or partitioning) of the superoxide (O₂⁻) radical into either ordinary molecular oxygen (O₂) or hydrogen peroxide (H₂O₂).

The invention is furthermore directed to an animal feed or animal feed additive comprising polypeptides of fungal origin having superoxide dismutase activity.

More particularly, according to the invention, the animal feed or animal feed additive comprising polypeptides of fungal origin having superoxide dismutase activity may comprise a polypeptide of fungal origin having superoxide dismutase activity selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

Alternatively defined, the polypeptide having superoxide dismutase activity may comprise an amino acid sequence selected from the group consisting of:
a. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1;
b. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2;
c. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3;
d. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4;
e. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9;
f. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10;
g. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11;
h. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 12;
i. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 13;
j. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 14;
k. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 15;
l. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 16;
m. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17;
n. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19;
o. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 20;
p. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21;
q. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22;
r. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23;
s. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 24;
t. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 sequence identity to the amino acid sequence of SEQ ID NO: 25;
u. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26;
v. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 27;
w. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 28;
x. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 29;
y. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 30;
z. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 31;
aa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 32;
bb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33;
cc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 34;
dd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 36;
ee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 38;
ff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 39;
gg. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 42;
jj. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43;
kk. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44;
ll. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 45;
mm. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 46;
nn. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 47; and
oo. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 48;
pp. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:199;
qq. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:200;
rr. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO 201;
ss. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:202;
tt. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:203;
uu. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:204;
vv. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 205;
ww. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:206;
xx. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:207;
yy. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:208;
zz. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:209;
aaa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:232;
bbb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:233;
ccc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:234;
ddd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:235;
eee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:236; and
fff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:237.

The polypeptide of fungal origin having superoxide dismutase activity may be selected from the group consisting
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

The polypeptide of fungal origin having superoxide dismutase activity may be selected from the group consisting of
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 307 of SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 206 of SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 154 of SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 188 of SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 184 of SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 249 of SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 178 of SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 248 of SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 274 of SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 26 to 257 of SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 261 of SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 269 of SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 151 of SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 228 of SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 37 to 233 of SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 227 of SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 34 to 230 of SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 233 of SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 25 to 227 of SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 211 of SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 218 of SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 218 of SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 182 of SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 185 of SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 185 of SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 304 of SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 310 of SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 303 of SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 209 of SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 209 of SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 22 to 183 of SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 193 of SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 177 of SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 215 of SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 182 of SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 154 of SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 150 of SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 247 of SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 154 of SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 316 of SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 193 of SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 14 to 164 of SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 184 of SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 242 of SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 261 of SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 258 of SEQ ID NO 243.

The polypeptide of fungal origin having superoxide dismutase activity may be selected from the group consisting
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.

As the SODs defined above are novel enzymes, the invention further relates to an isolated polypeptide as defined herein having superoxide dismutase activity.

The amino acid changes in the sequences disclosed above may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in the polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for SOD activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Sources of Superoxide Dismutases

The polypeptide of the invention having superoxide dismutase activity is of fungal origin.

As shown from the Examples, the superoxide dismutase of the invention may be obtainable, may be obtained, may be derived from a superoxide dismutase obtainable from a fungus selected from the group consisting of Trichoderma reesei, Aspergillus versicolor, Aspergillus deflectus, Aspergillus egyptiacus, Westerdykella sp. AS85-2, Aspergillus sp. XZ2669, Preussia terricola, Kionochaeta sp., Metapochonia bulbillosa, Xylomelasma sp. XZ0718, Preussia flanaganii, Cladobotryum sp., Westerdykella sp-46156, Trichoderma hamatum, Mycothermus thermophilus, Cephalotrichiella penicillate, Chaetomium megalocarpum, Chaetomium thermophilum var. thermophilum, Humicola hyalothermophila, Subramaniula anamorphosa, Sphingobacterium sp. T2, Trichoderma rossicum, Trichoderma lixii, Trichoderma sp-54723, Aspergillus niveus, Aspergillus templicola, Pochonia chlamydosporia var. spinulospora, Trichoderma sp-44174, Trichoderma rossicum, Trichoderma sp-54723, Trichoderma sp-44174, Metapochonia suchlasporia, Metarhizium marquandii, Diaporthe nobilis, Tolypocladium sp. XZ2627, Aspergillus japonicus, Metarhizium sp. XZ2431, Armillaria ostoyae, Trichoderma spirale, Aspergillus elegans, Trichoderma sinuosum, Trichoderma virens, Trichoderma harzianum, Fusicolla acetilerea, Plectosphaerella sp. 1-29, Mariannaea punicea, Penicillium oxalicum, Colletotrichum sp-71086, Aspergillus sp. nov. XZ3202, Trichoderma parapiluliferum, Aspergillus sp. nov. XZ3202, Mucor sp. XZ2651, Rhizomucor miehei, Mucor sp. XZ2651, Amphisphaeriaceae-sp 43674, Humicola fuscoatra and Valsaria rubricosa.

The polypeptide having superoxide dismutase activity may be selected from the group consisting of:

a. a polypeptide Trichoderma reesei from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide from Cladobotryum sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide Mariannaea punicea from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

The polypeptide having superoxide dismutase activity may be selected from the group consisting
a. a polypeptide from Trichoderma reesei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide from Cladobotryum sp having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide from Mariannaea punicea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

### Metals of Superoxide Dismutases

In an embodiment of the invention the polypeptide having superoxide dismutase activity is of fungal origin and is selected from the group consisting of a Cu-SOD, a Zn-SOD, a Cu/Zn-SOD, a Mn-SOD, and an Fe-SOD, more typically a Cu-SOD, a Zn-SOD, a Cu/Zn-SOD, a Mn-SOD, and an Fe-SOD, even more typically a Cu-SOD, a Zn-SOD, a Cu/Zn-SOD, or a Mn-SOD.

In a suitable embodiment of the invention the polypeptide having superoxide dismutase activity is of fungal origin and is selected from the group consisting of a Cu-SOD and a Cu/Zn-SOD.

The copper ion is involved in the catalysis of Cu-SODs whereas the zinc ion plays a structural role. The redox active copper catalyzes the disproportionation of superoxide anion to oxygen and hydrogen peroxide, whereas the zinc helps stabilize the protein and promotes pH independence of the reaction.

Table of Metals

| | SEQ ID NO | SEQ ID NO | SEQ ID NO | |
|---|---|---|---|---|
| Source | Mature peptide | Full polypeptide | DNA | Metal |
| Trichoderma reesei | SED ID NO 1 | SEQ ID NO 83 | SEQ ID NO 5 | Cu |
| Aspergillus versicolor | SED ID NO 2 | SEQ ID NO 84 | SEQ ID NO 87 | Cu |
| Aspergillus deflectus | SEQ ID NO 3 | SEQ ID NO 85 | SEQ ID NO 88 | Cu/Zn |
| Aspergillus egyptiacus | SEQ ID NO 4 | SEQ ID NO 86 | SEQ ID NO 89 | Cu |
| Westerdykella sp. | SEQ ID NO 9 | SEQ ID NO 49 | SEQ ID NO 90 | Fe |
| AS85-2 | | | | |
| Aspergillus sp. XZ2669 | SEQ ID NO 10 | SEQ ID NO 50 | SEQ ID NO 91 | Cu |
| Preussia terricola | SEQ ID NO 11 | SEQ ID NO 51 | SEQ ID NO 92 | Fe |
| Kionochaeta sp. | SEQ ID NO 12 | SEQ ID NO 52 | SEQ ID NO 93 | Cu/Zn |
| Kionochaeta sp. | SEQ ID NO 13 | SEQ ID NO 53 | SEQ ID NO 94 | Cu/Zn |
| Metapochonia bulbillosa | SEQ ID NO 14 | SEQ ID NO 54 | SEQ ID NO 95 | Cu |
| Xylomelasma sp. | SEQ ID NO 15 | SEQ ID NO 55 | SEQ ID NO 96 | Cu |
| XZ0718 | | | | |
| Preussia flanaganii | SEQ ID NO 16 | SEQ ID NO 56 | SEQ ID NO 97 | Fe |
| Cladobotryum sp. | SEQ ID NO 17 | SEQ ID NO 18 | SEQ ID NO 98 | Fe |
| Westerdykella sp- | SEQ ID NO 19 | SEQ ID NO 57 | SEQ ID NO 99 | Fe |
| 46156 | | | | |
| Trichoderma hamatum | SEQ ID NO 20 | SEQ ID NO 58 | SEQ ID NO 100 | Fe |
| Mycothermus thermophilus | SEQ ID NO 21 | SEQ ID NO 59 | SEQ ID NO 101 | Cu/Zn |
| Cephalotrichiella penicillata | SEQ ID NO 22 | SEQ ID NO 60 | SEQ ID NO 102 | Fe |
| Chaetomium | SEQ ID NO 23 | SEQ ID NO 61 | SEQ ID NO 127 | Fe |
| megalocarpum | | | | |
| Chaetomium thermophilum | SEQ ID NO 24 | SEQ ID NO 62 | SEQ ID NO 128 | Fe |
| var. thermophilum | | | | |
| Humicola hyalothermophila | SEQ ID NO 25 | SEQ ID NO 63 | SEQ ID NO 129 | Fe |
| Subramaniula anamorphosa | SEQ ID NO 26 | SEQ ID NO 64 | SEQ ID NO 130 | Fe |
| Sphingobacterium sp. | SEQ ID NO 27 | SEQ ID NO 65 | SEQ ID NO 131 | Fe |
| T2 | | | | |
| Trichoderma rossicum | SEQ ID NO 28 | SEQ ID NO 66 | SEQ ID NO 132 | Cu |
| Trichoderma lixii | SEQ ID NO 29 | SEQ ID NO 67 | SEQ ID NO 133 | Cu |
| Trichoderma sp-54723 | SEQ ID NO 30 | SEQ ID NO 68 | SEQ ID NO 134 | Cu |
| Aspergillus niveus | SEQ ID NO 31 | SEQ ID NO 69 | SEQ ID NO 135 | Cu |
| Aspergillus templicola | SEQ ID NO 32 | SEQ ID NO 70 | SEQ ID NO 136 | Cu |
| Pochonia chlamydosporia | SEQ ID NO 33 | SEQ ID NO 71 | SEQ ID NO 137 | Cu |
| var. spinulospora | | | | |
| Trichoderma sp-44174 | SEQ ID NO 34 | SEQ ID NO 35 | SEQ ID NO 138 | Cu |
| Trichoderma rossicum | SEQ ID NO 36 | SEQ ID NO 37 | SEQ ID NO 139 | Cu |
| Trichoderma sp-54723 | SEQ ID NO 38 | SEQ ID NO 72 | SEQ ID NO 140 | Cu |
| Trichoderma sp-44174 | SEQ ID NO 39 | SEQ ID NO 73 | SEQ ID NO 141 | Cu |
| Metapochonia suchlasporia | SEQ ID NO 40 | SEQ ID NO 74 | SEQ ID NO 142 | Cu |
| Metarhizium marquandii | SEQ ID NO 41 | SEQ ID NO 75 | SEQ ID NO 143 | Cu |
| Diaporthe nobilis | SEQ ID NO 42 | SEQ ID NO 76 | SEQ ID NO 144 | Cu |
| Tolypocladium sp. | SEQ ID NO 43 | SEQ ID NO 77 | SEQ ID NO 145 | Cu/Zn |
| XZ2627 | | | | |
| Aspergillus japonicus | SEQ ID NO 44 | SEQ ID NO 78 | SEQ ID NO 146 | Cu |
| Metarhizium sp. | SEQ ID NO 45 | SEQ ID NO 79 | SEQ ID NO 147 | Cu |
| XZ2431 | | | | |
| Armillaria ostoyae | SEQ ID NO 46 | SEQ ID NO 80 | SEQ ID NO 148 | Cu/Zn |
| Trichoderma spirale | SEQ ID NO 47 | SEQ ID NO 81 | SEQ ID NO 149 | Cu |
| Aspergillus elegans | SEQ ID NO 48 | SEQ ID NO 82 | SEQ ID NO 150 | Cu/Zn |
| Trichoderma sinuosum | SEQ ID NO 199 | SEQ ID NO 210 | SEQ ID NO 221 | Cu |
| Trichoderma virens | SEQ ID NO 200 | SEQ ID NO 211 | SEQ ID NO 222 | Cu/Zn |
| Trichoderma | SEQ ID NO 201 | SEQ ID NO 212 | SEQ ID NO 223 | Cu |
| harzianum | | | | |
| Fusicolla acetilerea | SEQ ID NO 202 | SEQ ID NO 213 | SEQ ID NO 224 | Cu/Zn |
| Plectosphaerella sp. 1- | SEQ ID NO 203 | SEQ ID NO 214 | SEQ ID NO 225 | Cu/Zn |
| 29 | | | | |
| Mariannaea punicea | SEQ ID NO 204 | SEQ ID NO 215 | SEQ ID NO 226 | Cu/Zn |
| Penicillium oxalicum | SEQ ID NO 205 | SEQ ID NO 216 | SEQ ID NO 227 | Cu/Zn |
| Colletotrichum sp- | SEQ ID NO 206 | SEQ ID NO 217 | SEQ ID NO 228 | Cu |
| 71086 | | | | |
| Aspergillus sp. nov. | SEQ ID NO 207 | SEQ ID NO 218 | SEQ ID NO 229 | Cu/Zn |
| XZ3202 | | | | |
| Trichoderma | SEQ ID NO 208 | SEQ ID NO 219 | SEQ ID NO 230 | Cu |
| parapiluliferum | | | | |
| Aspergillus sp. nov. | SEQ ID NO 209 | SEQ ID NO 220 | SEQ ID NO 231 | Cu |
| XZ3202 | | | | |
| Mucor sp. XZ2651 | SEQ ID NO 232 | SEQ ID NO 238 | SEQ ID NO 244 | Cu |
| Rhizomucor miehei | SEQ ID NO 233 | SEQ ID NO 239 | SEQ ID NO 245 | Cu |
| Mucor sp. XZ2651 | SEQ ID NO 234 | SEQ ID NO 240 | SEQ ID NO 246 | Cu |
| Amphisphaeriaceae | SEQ ID NO 235 | SEQ ID NO 241 | SEQ ID NO 247 | Cu |
| sp-43674 | | | | |
| Humicola fuscoatra | SEQ ID NO 236 | SEQ ID NO 242 | SEQ ID NO 248 | Cu/Zn |
| Valsaria rubricosa | SEQ ID NO 237 | SEQ ID NO 243 | SEQ ID NO 249 | Cu |

### Catalytic Site of Superoxide Dismutases

In an embodiment of the invention, the polypeptide having superoxide dismutase activity has a conserved catalytic site selected from the group consisting of H69, H71, H86, H150; H66, H68, H84, H148; H66, H68, H84, H148; H68, H70, H85, H149; H69, H71, H86, H150: H68, H70, H86, H150; H62, H64, H80, H144; H44, H46, H61, H118; H79, H81, H97, H161: H82, H84, H100, H164; H65, H67, H83, H150; H45, H47, H62, H119; H67, H69, H85, H149; H50, H52, H67, H126; H67, H69, H85, H149; H63, H65, H81, H148; H67, H69, H85, H149; H62, H64, H80, H147; H62, H64, H80, H147; H68, H70, H85, H149; H69, H71, H86, H150; H75, H77, H93, H156; H61, H63, H79, H146; H68, H70, H85, H149.
In a suitable embodiment, the SOD of the invention is a polypeptide having super oxide dismutase activity is selected from the group consisting of
i. a polypeptide which has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 34, comprises amino acid residues H69, H71, H86, H150 at its catalytic site and suitably obtainable from Trichoderma sp-44174;
ii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 39, comprises amino acid residues H69, H71, H86, H150 at its catalytic site and suitably obtainable from Trichoderma sp-44174;
iii. a polypeptide which invention has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 33, comprises amino acid residues H66, H68, H84, H148 at its catalytic site and suitably obtainable from Pochonia chlamydosporia var. spinulospora. iv. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 37, comprises amino acid residues H68, H70, H85, H149 at its catalytic site and suitably obtainable from Trichoderma sp-54723;
v. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 28, comprises amino acid residues H69, H71, H86, H150 at its catalytic site and suitably obtainable from Trichoderma rossicum;
vi. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 45, comprises amino acid residues H68, H70, H86, H150 at its catalytic site and suitably obtainable from Metarhizium sp. XZ2431;
vii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 29, comprises amino acid residues H69, H71, H86, H150 at its catalytic site and suitably obtainable from Trichoderma lixii;
viii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 12, comprises amino acid residues H69, H71, H86, H150 at its catalytic site and suitably obtainable from Kionochaeta sp;
ix. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 21, comprises amino acid residues H44, H46, H61, H118 at its catalytic site and suitably obtainable from Mycothermus thermophilus;
x. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 30, comprises amino acid residues H68, H70, H85, H149 at its catalytic site and suitably obtainable from Trichoderma sp-54723;
xi. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 13, comprises amino acid residues H79, H81, H97, H161 at its catalytic site and suitably obtainable from Kionochaeta sp;
xii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 15, comprises amino acid residues H82, H84, H100, H164 at its catalytic site and suitably obtainable from Xylomelasma sp. XZ0718;
xiii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 48, comprises amino acid residues H65, H67, H83, H150 at its catalytic site and suitably obtainable from Aspergillus elegans;
xiv. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 14, comprises amino acid residues H67, H69, H85, H149 at its catalytic site and suitably obtainable from Metapochonia bulbillosa;
xv. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 46, comprises amino acid residues H50, H52, H67, H126 at its catalytic site and suitably obtainable from Armillaria ostoyae;
xvi. a polypeptide which invention has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 40, comprises amino acid residues H67, H69, H85, H149 at its catalytic site and suitably obtainable from Metapochonia suchlasporia;
xvii. a polypeptide which as a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 31, comprises amino acid residues H63, H65, H81, H148 at its catalytic site and suitably obtainable from Aspergillus niveus;
xviii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 41, comprises amino acid residues H67, H69, H85, H149 at its catalytic site and suitably obtainable from Metarhizium marquandii;
xix. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 10, comprises amino acid residues H62, H64, H80, H147 at its catalytic site and suitably obtainable from Aspergillus sp. XZ2669;
xx. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 32, comprises amino acid residues H62, H64, H80, H147 at its catalytic site and suitably obtainable from Aspergillus templicola;
xxi. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 35, comprises amino acid residues H69, H71, H86, H150 at its catalytic site and suitably obtainable from Trichoderma rossicum;
xxii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 42, comprises amino acid residues H75, H77, H93, H156 at its catalytic site and suitably obtainable from Diaporthe nobilis;
xxiii. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 44, comprises amino acid residues H61, H63, H79, H146 at its catalytic site and suitably obtainable from Aspergillus japonicus;
xxiv. a polypeptide which has a at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 43, comprises amino acid residues H68, H70, H85, H149 at its catalytic site and suitably obtainable from Tolypocladium sp. XZ2627; and
xxv. a polypeptide which has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the Cu-SOD of SEQ ID NO: 47, comprises amino acid residues H69, H71, H86, H150 at its catalytic site and suitably obtainable from Trichoderma spirale.

### Activity of Superoxide Dismutases

In a preferred embodiment, the SOD of the invention has an activity level above 1000 U/mg EP when measured according to the method described in Example 9 at pH 7.8. Accordingly, in one embodiment of the invention the SOD is selected from a polypeptide which has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a mature polypeptide selected from the group consisting of SEQ ID NO 1, SEQ ID NO 15, SEQ ID NO 21, SEQ ID NO 24, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 39, SEQ ID NO 41, SEQ ID NO 42, SEQ ID NO 44, SEQ ID NO 45, SEQ ID NO 48, SEQ ID NO 202, SEQ ID NO 203, SEQ ID NO 204, SEQ ID NO 205, SEQ ID NO 206, SEQ ID NO 207, SEQ ID NO 208, SEQ ID NO 209, SEQ ID NO 232, SEQ ID NO 233, SEQ ID NO 234, SEQ ID NO 235, SEQ ID NO 236, SEQ ID NO 237 or with a full length polypeptide selected from the group consisting of SEQ ID NO 83, SEQ ID NO 50, SEQ ID NO 51, SEQ ID NO 52, SEQ ID NO 53, SEQ ID NO 54, SEQ ID NO 55, SEQ ID NO 56, SEQ ID NO 18, SEQ ID NO 57, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 35, SEQ ID NO 37, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 210, SEQ ID NO 211, SEQ ID NO 212, SEQ ID NO 213, SEQ ID NO 214, SEQ ID NO 215, SEQ ID NO 216, SEQ ID NO 217, SEQ ID NO 218, SEQ ID NO 219, SEQ ID NO 220, SEQ ID NO 238, SEQ ID NO 239, SEQ ID NO 240, SEQ ID NO 241, SEQ ID NO 242 and SEQ ID NO 243.

Activity Table

| | Mature polypeptide | Full polypeptide | Relative Activity |
|---|---|---|---|
| Source | SEQ ID NO | SEQ ID NO | Unit (U/mg EP) |
| Trichoderma reesei | SED ID NO 1 | SEQ ID NO 83 | 6055 |
| Westerdykella sp. AS85-2 | SEQ ID NO 9 | SEQ ID NO 49 | 699 |
| Aspergillus sp. XZ2669 | SEQ ID NO 10 | SEQ ID NO 50 | 1000 |
| Preussia terricola | SEQ ID NO 11 | SEQ ID NO 51 | 634 |
| Kionochaeta sp. | SEQ ID NO 12 | SEQ ID NO 52 | 1184 |
| Kionochaeta sp. | SEQ ID NO 13 | SEQ ID NO 53 | 2108 |
| Metapochonia bulbillosa | SEQ ID NO 14 | SEQ ID NO 54 | 1494 |
| Xylomelasma sp. XZ0718 | SEQ ID NO 15 | SEQ ID NO 55 | 3900 |
| Preussia flanaganii | SEQ ID NO 16 | SEQ ID NO 56 | 1451 |
| Cladobotryum sp. | SEQ ID NO 17 | SEQ ID NO 18 | na |
| Westerdykella sp-46156 | SEQ ID NO 19 | SEQ ID NO 57 | 1309 |
| Trichoderma hamatum | SEQ ID NO 20 | SEQ ID NO 58 | 388 |
| Mycothermus thermophilus | SEQ ID NO 21 | SEQ ID NO 59 | 2678 |
| Cephalotrichiella penicillata | SEQ ID NO 22 | SEQ ID NO 60 | 1034 |
| Chaetomium megalocarpum | SEQ ID NO 23 | SEQ ID NO 61 | 1641 |
| Chaetomium thermophilum | SEQ ID NO 24 | SEQ ID NO 62 | 2451 |
| var. thermophilum | | | |
| Humicola hyalothermophila | SEQ ID NO 25 | SEQ ID NO 63 | 1293 |
| Subramaniula anamorphosa | SEQ ID NO 26 | SEQ ID NO 64 | na |
| Sphingobacterium sp. T2 | SEQ ID NO 27 | SEQ ID NO 65 | 279 |
| Trichoderma rossicum | SEQ ID NO 28 | SEQ ID NO 66 | 1889 |
| Trichoderma lixii | SEQ ID NO 29 | SEQ ID NO 67 | 2701 |
| Trichoderma sp-54723 | SEQ ID NO 30 | SEQ ID NO 68 | 2662 |
| Aspergillus niveus | SEQ ID NO 31 | SEQ ID NO 69 | 3646 |
| Aspergillus templicola | SEQ ID NO 32 | SEQ ID NO 70 | 5483 |
| Pochonia chlamydosporia | SEQ ID NO 33 | SEQ ID NO 71 | 2026 |
| var. Spinulospora | | | |
| Trichoderma sp-44174 | SEQ ID NO 34 | SEQ ID NO 35 | 2937 |
| Trichoderma rossicum | SEQ ID NO 36 | SEQ ID NO 37 | 2615 |
| Trichoderma sp-54723 | SEQ ID NO 38 | SEQ ID NO 72 | 1298 |
| Trichoderma sp-44174 | SEQ ID NO 39 | SEQ ID NO 73 | 2314 |
| Metapochonia suchlasporia | SEQ ID NO 40 | SEQ ID NO 74 | 1938 |
| Metarhizium marquandii | SEQ ID NO 41 | SEQ ID NO 75 | 2808 |
| Diaporthe nobilis | SEQ ID NO 42 | SEQ ID NO 76 | 4348 |
| Tolypocladium sp. | SEQ ID NO 43 | SEQ ID NO 77 | 2052 |
| XZ2627 | | | |
| Aspergillus japonicus | SEQ ID NO 44 | SEQ ID NO 78 | 7754 |
| Metarhizium sp. XZ2431 | SEQ ID NO 45 | SEQ ID NO 79 | 6941 |
| Armillaria ostoyae | SEQ ID NO 46 | SEQ ID NO 80 | 1877 |
| Trichoderma spirale | SEQ ID NO 47 | SEQ ID NO 81 | 1290 |
| Aspergillus elegans | SEQ ID NO 48 | SEQ ID NO 82 | 2651 |
| Trichoderma sinuosum | SEQ ID NO 199 | SEQ ID NO 210 | 328 |
| Trichoderma virens | SEQ ID NO 200 | SEQ ID NO 211 | 684 |
| Trichoderma harzianum | SEQ ID NO 201 | SEQ ID NO 212 | 654 |
| Fusicolla acetilerea | SEQ ID NO 202 | SEQ ID NO 213 | 9527 |
| Plectosphaerella sp. 1-29 | SEQ ID NO 203 | SEQ ID NO 214 | 5952 |
| Mariannaea punicea | SEQ ID NO 204 | SEQ ID NO 215 | 4410 |
| Penicillium oxalicum | SEQ ID NO 205 | SEQ ID NO 216 | 16555 |
| Colletotrichum sp-71086 | SEQ ID NO 206 | SEQ ID NO 217 | 2483 |
| Aspergillus sp. nov. | SEQ ID NO 207 | SEQ ID NO 218 | 4018 |
| XZ3202 | | | |
| Trichoderma | SEQ ID NO 208 | SEQ ID NO 219 | 2525 |
| parapiluliferum | | | |
| Aspergillus sp. nov. | SEQ ID NO 209 | SEQ ID NO 220 | 5287 |
| XZ3202 | | | |
| Mucor sp. XZ2651 | SEQ ID NO 232 | SEQ ID NO 238 | 3,762 |
| Rhizomucor miehei | SEQ ID NO 233 | SEQ ID NO 239 | 4,406 |
| Mucor sp. XZ2651 | SEQ ID NO 234 | SEQ ID NO 240 | 8,182 |
| Amphisphaeriaceae sp- | SEQ ID NO 235 | SEQ ID NO 241 | 3,018 |
| 43674 | | | |
| Humicola fuscoatra | SEQ ID NO 236 | SEQ ID NO 242 | 5,744 |
| Valsaria rubricosa | SEQ ID NO 237 | SEQ ID NO 243 | 2,977 |

In one embodiment of the invention, the superoxide dismutase of fungal origin has an activity of at least 1000 U/mg at pH 7.8, such as at least 1500 U/mg, or at least 2000 U/mg, or at least 3000 U/mg, or at least 4000 U/mg, or at least 5000 U/mg, suitably as measured by the method described in Example 9.

SEQ ID NO 1, SEQ ID NO 9, SEQ ID NO 15, SEQ ID NO 21, SEQ ID NO 24, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 39, SEQ ID NO 41, SEQ ID NO 42, SEQ ID NO 44, SEQ ID NO 45, and SEQ ID NO 48 all have an activity near, at or over 3000 U/mg EP when measured according to the method described in Example 9 or at pH 7.8.

### Gastric Stability of Superoxide Dismutases

In a preferred embodiment, the SOD, given it is intended for use as an animal feed additive, is gastric stable, when measured according to the method described in Example 10. As can be seen from the table in Example 10, the commercially available superoxide dismutase from bovine erythrocytes and expressed in E. coli of sequence A:

MATKAVCVLKGDGPVQGTIHFEAKGDTVVVTGSITGLTEGDHGFHVHQFG
DNTQGCTSAGPHFNPLSKKHGGPKDEERHVGDLGNVTADKNGVAIVDIVD
PLISLSGEYSIIGRTMVVHEKPDDLGRGGNEESTKTGNAGSRLACGVIGI
AK

retains only 26% of its activity upon gastric treatment in the presence of pepsin.

Two commercial SODs were tested as references: SOD-Mn, from E. coli Sigma S5639 with a activity of 10240 U/mg EP and only 26% activity left after gastric press for 30 min; and SOD-Cu/Zn from Bovine expressed in E. coli Sigma S9697 with activity of 6108 U/mg EP and only 35% activity left after gastric press for 30 min.

The fungal SODs of the invention were overwhelmingly and surprisingly found to have a very high gastric stability (see Gastric Stability Table). Moreover, some SODs even have higher activity under gastric stability studies than at the reference pH, indicating that they are more active at low pH. This makes fungal SODs very well suited for use in vivo by means of administration through animal feed.

Gastric Stability Table

| | | | Gastric stability after |
|---|---|---|---|
| | SEQ ID NO | SEQ ID NO | 15 min (remaining |
| Source | Mature peptide | Full polypeptide | activity, approx, %) |
| Trichoderma reesei | SED ID NO 1 | SEQ ID NO 83 | 100 |
| Preussia terricola | SEQ ID NO 11 | SEQ ID NO 51 | 70 |
| Kionochaeta sp. | SEQ ID NO 12 | SEQ ID NO 52 | 88 |
| Kionochaeta sp. | SEQ ID NO 13 | SEQ ID NO 53 | 92 |
| Metapochonia bulbillosa | SEQ ID NO 14 | SEQ ID NO 54 | 100 |
| Xylomelasma sp. XZ0718 | SEQ ID NO 15 | SEQ ID NO 55 | 100 |
| Preussia flanaganii | SEQ ID NO 16 | SEQ ID NO 56 | 90 |
| Cladobotryum sp. | SEQ ID NO 17 | SEQ ID NO 18 | 100 |
| Westerdykella sp-46156 | SEQ ID NO 19 | SEQ ID NO 57 | 52 |
| Trichoderma hamatum | SEQ ID NO 20 | SEQ ID NO 58 | 98 |
| Mycothermus thermophilus | SEQ ID NO 21 | SEQ ID NO 59 | 40 |
| Cephalotrichiella penicillata | SEQ ID NO 22 | SEQ ID NO 60 | 85 |
| Chaetomium megalocarpum | SEQ ID NO 23 | SEQ ID NO 61 | 45 |
| Chaetomium thermophilum | SEQ ID NO 24 | SEQ ID NO 62 | 57 |
| var. Thermophilum | | | |
| Humicola hyalothermophila | SEQ ID NO 25 | SEQ ID NO 63 | 62 |
| Subramaniula anamorphosa | SEQ ID NO 26 | SEQ ID NO 64 | 97 |
| Sphingobacterium sp. T2 | SEQ ID NO 27 | SEQ ID NO 65 | 78 |
| Trichoderma rossicum | SEQ ID NO 28 | SEQ ID NO 66 | 128 |
| Trichoderma lixii | SEQ ID NO 29 | SEQ ID NO 67 | 144 |
| Trichoderma sp-54723 | SEQ ID NO 30 | SEQ ID NO 68 | 124 |
| Aspergillus niveus | SEQ ID NO 31 | SEQ ID NO 69 | 126 |
| Aspergillus templicola | SEQ ID NO 32 | SEQ ID NO 70 | 90 |
| Pochonia chlamydosporia var. | SEQ ID NO 33 | SEQ ID NO 71 | 105 |
| Spinulospora | | | |
| Trichoderma sp-44174 | SEQ ID NO 34 | SEQ ID NO 35 | 120 |
| Trichoderma rossicum | SEQ ID NO 36 | SEQ ID NO 37 | 96 |
| Trichoderma sp-54723 | SEQ ID NO 38 | SEQ ID NO 72 | 100 |
| Trichoderma sp-44174 | SEQ ID NO 39 | SEQ ID NO 73 | 104 |
| Metapochonia suchlasporia | SEQ ID NO 40 | SEQ ID NO 74 | 83 |
| Metarhizium marquandii | SEQ ID NO 41 | SEQ ID NO 75 | 118 |
| Diaporthe nobilis | SEQ ID NO 42 | SEQ ID NO 76 | 98 |
| Tolypocladium sp. XZ2627 | SEQ ID NO 43 | SEQ ID NO 77 | 109 |
| Aspergillus japonicus | SEQ ID NO 44 | SEQ ID NO 78 | 104 |
| Metarhizium sp. XZ2431 | SEQ ID NO 45 | SEQ ID NO 79 | 104 |
| Armillaria ostoyae | SEQ ID NO 46 | SEQ ID NO 80 | 79 |
| Trichoderma spirale | SEQ ID NO 47 | SEQ ID NO 81 | 41 |
| Aspergillus elegans | SEQ ID NO 48 | SEQ ID NO 82 | 61 |
| Trichoderma sinuosum | SEQ ID NO 199 | SEQ ID NO 210 | 99 |
| Trichoderma virens | SEQ ID NO 200 | SEQ ID NO 211 | 73 |
| Trichoderma harzianum | SEQ ID NO 201 | SEQ ID NO 212 | 80 |
| Fusicolla acetilerea | SEQ ID NO 202 | SEQ ID NO 213 | 25 |
| Plectosphaerella sp. 1-29 | SEQ ID NO 203 | SEQ ID NO 214 | 23 |
| Mariannaea punicea | SEQ ID NO 204 | SEQ ID NO 215 | 23 |
| Penicillium oxalicum | SEQ ID NO 205 | SEQ ID NO 216 | 30 |
| Colletotrichum sp-71086 | SEQ ID NO 206 | SEQ ID NO 217 | 81 |
| Aspergillus sp. nov. XZ3202 | SEQ ID NO 207 | SEQ ID NO 218 | 72 |
| Trichoderma parapiluliferum | SEQ ID NO 208 | SEQ ID NO 219 | 97 |
| Aspergillus sp. nov. XZ3202 | SEQ ID NO 209 | SEQ ID NO 220 | 92 |
| Mucor sp. XZ2651 | SEQ ID NO 232 | SEQ ID NO 238 | 99 |
| Rhizomucor miehei | SEQ ID NO 233 | SEQ ID NO 239 | 90 |
| Mucor sp. XZ2651 | SEQ ID NO 234 | SEQ ID NO 240 | 113 |
| Amphisphaeriaceae sp- | SEQ ID NO 235 | SEQ ID NO 241 | 34 |
| 43674 | | | |
| Humicola fuscoatra | SEQ ID NO 236 | SEQ ID NO 242 | 57 |
| Valsaria rubricosa | SEQ ID NO 237 | SEQ ID NO 243 | 72 |
| S5639 from Sigma Mn-SOD | | | 26 |
| Escherichia coli | | | |
| S9697 Bovine Cu/Zn SOD | | | 35 |
| SIGMA | | | |

One aspect of the invention is directed to an animal feed additive comprising a superoxide dismutase of fungal origin, wherein the superoxide dismutase retains 40% activity, at least 50% activity, such as retains at least 55% activity, such as retains at least 60% activity, such as retains at least 65% activity, such as retains at least 70% activity, such as retaining at least 75% activity, such as retains at least 80% activity, most preferably at least 85% activity, or 90%, or 95% activity under gastric stability studies as measure by the method of Example 10.

One aspect of the invention is directed to an animal feed additive comprising a fungal superoxide dismutase wherein the superoxide dismutase has improved gastric stability compared to sequence A, such that the superoxide dismutase is gastric stable such that it retains at least 40% of its activity when measured according to the test method described in Example 10, such as retaining at least 50% activity, such as retaining at least 55% activity, such as retaining at least 60% activity, such as retaining at least 65% activity, such as retaining at least 70% activity, such as retaining at least 75% activity, such as retaining at least 80% activity.

As can be seen from the Table in Example 10 and the Gastric Stability Table, each of SEQ ID NO 1, SEQ ID NO 83, SEQ ID NO 11, SEQ ID NO 51, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 19, SEQ ID NO 57, SEQ ID NO 20, SEQ ID NO 58, SEQ ID NO 21, SEQ ID NO 59, SEQ ID NO 22, SEQ ID NO 60, SEQ ID NO 23, SEQ ID NO 61, SEQ ID NO 24, SEQ ID NO 62, SEQ ID NO 25, SEQ ID NO 63, SEQ ID NO 26, SEQ ID NO 64, SEQ ID NO 27, SEQ ID NO 65, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 47, SEQ ID NO 81, SEQ ID NO 48, SEQ ID NO 82, SEQ ID NO 199, SEQ ID NO 210, SEQ ID NO 200, SEQ ID NO 211, SEQ ID NO 201, SEQ ID NO 212, SEQ ID NO 206, SEQ ID NO 217, SEQ ID NO 207, SEQ ID NO 218, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234, SEQ ID NO 240, SEQ ID NO 236, SEQ ID NO 242, SEQ ID NO 237 and SEQ ID NO 243 retain at least 40% of their activity under gastric stability studies, when measured according to the test method described in Example 10.

Accordingly, in one embodiment the polypeptide having superoxide dismutase activity may be selected a polypeptide having at least 80% sequence identity, such as at least 85% sequence identity, such as at least 90% sequence identity, such as at least 95% sequence identity, such as at least 98% sequence identity, such as at least 99% sequence identity of a polypeptide selected from the group consisting of SEQ ID NO 1, SEQ ID NO 83, SEQ ID NO 11, SEQ ID NO 51, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 19, SEQ ID NO 57, SEQ ID NO 20, SEQ ID NO 58, SEQ ID NO 21, SEQ ID NO 59, SEQ ID NO 22, SEQ ID NO 60, SEQ ID NO 23, SEQ ID NO 61, SEQ ID NO 24, SEQ ID NO 62, SEQ ID NO 25, SEQ ID NO 63, SEQ ID NO 26, SEQ ID NO 64, SEQ ID NO 27, SEQ ID NO 65, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 47, SEQ ID NO 81, SEQ ID NO 48, SEQ ID NO 82, SEQ ID NO 199, SEQ ID NO 210, SEQ ID NO 200, SEQ ID NO 211, SEQ ID NO 201, SEQ ID NO 212, SEQ ID NO 206, SEQ ID NO 217, SEQ ID NO 207, SEQ ID NO 218, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234, SEQ ID NO 240, SEQ ID NO 236, SEQ ID NO 242, SEQ ID NO 237 and SEQ ID NO 243. Typically, these SODs retain at least 40% of their activity under gastric stability studies, when measured according to the test method described in Example 10.

In a more preferred embodiment, the fungal SOD of the invention retains at least 50% of its activity under gastric stability studies. Accordingly, in more preferred embodiment, the polypeptide having superoxide dismutase activity may be selected a polypeptide having at least 80% sequence identity, such as at least 85% sequence identity, such as at least 90% sequence identity, such as at least 95% sequence identity, such as at least 98% sequence identity, such as at least 99% sequence identity of a polypeptide selected from the group consisting of SEQ ID NO 1, SEQ ID NO 83, SEQ ID NO 11, SEQ ID NO 51, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 19, SEQ ID NO 57, SEQ ID NO 20, SEQ ID NO 58, SEQ ID NO 22, SEQ ID NO 60, SEQ ID NO 24, SEQ ID NO 62, SEQ ID NO 25, SEQ ID NO 63, SEQ ID NO 26, SEQ ID NO 64, SEQ ID NO 27, SEQ ID NO 65, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 48, SEQ ID NO 82, SEQ ID NO 199, SEQ ID NO 210, SEQ ID NO 200, SEQ ID NO 211, SEQ ID NO 201, SEQ ID NO 212, SEQ ID NO 206, SEQ ID NO 217, SEQ ID NO 207, SEQ ID NO 218, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234, SEQ ID NO 240, SEQ ID NO 236, SEQ ID NO 242, SEQ ID NO 237 and SEQ ID NO 243. Typically, these SODs retain at least 50% of their activity under gastric stability studies, when measured according to the test method described in Example 10.

In a more preferred embodiment, the fungal SOD of the invention retains at least 60% of its activity under gastric stability studies. Accordingly, in more preferred embodiment, the polypeptide having superoxide dismutase activity may be selected a polypeptide having at least 80% sequence identity, such as at least 85% sequence identity, such as at least 90% sequence identity, such as at least 95% sequence identity, such as at least 98% sequence identity, such as at least 99% sequence identity of a polypeptide selected from the group consisting of SEQ ID NO 1, SEQ ID NO 83, SEQ ID NO 11, SEQ ID NO 51, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 20, SEQ ID NO 58, SEQ ID NO 22, SEQ ID NO 60, SEQ ID NO 25, SEQ ID NO 63, SEQ ID NO 26, SEQ ID NO 64, SEQ ID NO 27, SEQ ID NO 65, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 48, SEQ ID NO 82, SEQ ID NO 199, SEQ ID NO 210, SEQ ID NO 200, SEQ ID NO 211, SEQ ID NO 201, SEQ ID NO 212, SEQ ID NO 206, SEQ ID NO 217, SEQ ID NO 207, SEQ ID NO 218, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234, SEQ ID NO 240, SEQ ID NO 237 and SEQ ID NO 243. Typically, these SODs retain at least 60% of their activity under gastric stability studies, when measured according to the test method described in Example 10.

In a more preferred embodiment, the fungal SOD of the invention retains at least 70% of its activity under gastric stability studies. Accordingly, in more preferred embodiment, the polypeptide having superoxide dismutase activity may be selected a polypeptide having at least 80% sequence identity, such as at least 85% sequence identity, such as at least 90% sequence identity, such as at least 95% sequence identity, such as at least 98% sequence identity, such as at least 99% sequence identity of a polypeptide selected from the group consisting of SEQ ID NO 1, SEQ ID NO 83, SEQ ID NO 11, SEQ ID NO 51, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 20, SEQ ID NO 58, SEQ ID NO 22, SEQ ID NO 60, SEQ ID NO 26, SEQ ID NO 64, SEQ ID NO 27, SEQ ID NO 65, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 199, SEQ ID NO 210, SEQ ID NO 200, SEQ ID NO 211, SEQ ID NO 201, SEQ ID NO 212, SEQ ID NO 206, SEQ ID NO 217, SEQ ID NO 207, SEQ ID NO 218, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234, SEQ ID NO 240, SEQ ID NO 237 and SEQ ID NO 243. Typically, these SODs retain at least 70% of their activity under gastric stability studies, when measured according to the test method described in Example 10.

In a more preferred embodiment, the fungal SOD of the invention retains at least 80% of its activity under gastric stability studies. Accordingly, in more preferred embodiment, the polypeptide having superoxide dismutase activity may be selected a polypeptide having at least 80% sequence identity, such as at least 85% sequence identity, such as at least 90% sequence identity, such as at least 95% sequence identity, such as at least 98% sequence identity, such as at least 99% sequence identity of a polypeptide selected from the group consisting of SEQ ID NO 1, SEQ ID NO 83, SEQ ID NO 11, SEQ ID NO 12, SEQ ID NO 52, SEQ ID NO 13, SEQ ID NO 53, SEQ ID NO 14, SEQ ID NO 54, SEQ ID NO 15, SEQ ID NO 55, SEQ ID NO 16, SEQ ID NO 56, SEQ ID NO 17, SEQ ID NO 18, SEQ ID NO 20, SEQ ID NO 58, SEQ ID NO 22, SEQ ID NO 60, SEQ ID NO 26, SEQ ID NO 64, SEQ ID NO 28, SEQ ID NO 66, SEQ ID NO 29, SEQ ID NO 67, SEQ ID NO 30, SEQ ID NO 68, SEQ ID NO 31, SEQ ID NO 69, SEQ ID NO 32, SEQ ID NO 70, SEQ ID NO 33, SEQ ID NO 71, SEQ ID NO 34, SEQ ID NO 35, SEQ ID NO 36, SEQ ID NO 37, SEQ ID NO 38, SEQ ID NO 72, SEQ ID NO 39, SEQ ID NO 73, SEQ ID NO 40, SEQ ID NO 74, SEQ ID NO 41, SEQ ID NO 75, SEQ ID NO 42, SEQ ID NO 76, SEQ ID NO 43, SEQ ID NO 77, SEQ ID NO 44, SEQ ID NO 78, SEQ ID NO 45, SEQ ID NO 79, SEQ ID NO 46, SEQ ID NO 80, SEQ ID NO 199, SEQ ID NO 210, SEQ ID NO 206, SEQ ID NO 217, SEQ ID NO 208, SEQ ID NO 219, SEQ ID NO 209, SEQ ID NO 220, SEQ ID NO 232, SEQ ID NO 238, SEQ ID NO 233, SEQ ID NO 239, SEQ ID NO 234 and SEQ ID NO 240. Typically, these SODs retain at least 80% of their activity under gastric stability studies, when measured according to the test method described in Example 10.

In a combination of preferred embodiments, the superoxide dismutase is of fungal origin, and preferably has an activity of at least 1500 U/mg at pH 7.8 and is gastric stable such that it retains at least 70% of its activity under the gastric stability test. In an alternative combination of preferred embodiments, the superoxide dismutase is of fungal origin, and preferably has an activity of at least 5000 U/mg at pH 7.8 and is gastric stable such that it retains at least 20% of its activity under the gastric stability test.

Preferred polypeptide of fungal origin having superoxide dismutase activity have at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to polypeptides selected from the group consisting of SEQ ID NO 1, SEQ ID NO 83, SEQ ID NO 32, SEQ ID NO 70, SEQ ID NO 42, SEQ ID NO 76, SEQ ID NO 44, SEQ ID NO 78, SEQ ID NO 46, SEQ ID NO 80, SEQ ID NO 202 and SEQ ID NO 213.

### Thermal Stability of Superoxide Dismutase

The commercially available E. coli and bovine SODs are tested at 25° C. and stored at −20° C. An animal feed additive for use in animal feed is prepared by a pelleting process, transported by land or sea, stored, and consumed by animals with body temperatures over 30° C. and at gastric pH conditions. Transport and pelleting may lead to temperatures reaching 45° C. Accordingly, thermal stability in each of these conditions is highly pertinent for use of a super oxide dismutase in an animal feed additive.

Test were performed on the super oxide dismutases of the invention to measure the temperature at different acidity/alkalinity to determine the temperature at which the polypeptide unfolds and denatures. As can be seen, from the “Thermal Stability Table with pH”, unfolding of the fungal superoxide dismutase takes place at over 45° C., as measured by the Tm, for all fungal superoxide dismutases at neutral pH (pH7). A further embodiment is directed to an animal feed additive comprising a fungal superoxide dismutase thermally stable at pH 7, when measured according to the method described in Example 11, such that protein unfolding (Tm) is 45° C. or more.

At gastric pH (pH 3), fungal superoxide dismutases have been found to be stable at normal body temperatures for farm animals. A further embodiment of the invention is directed to an animal feed additive comprising a fungal superoxide dismutase thermally stable at pH 3, when measured according to the method described in Example 11, such that protein unfolding (Tm) is 35° C. or more, such as 40° C. or more, such as 45° C. or more.

In a preferred embodiment, the animal feed additive of the invention is thermal stable, when measured according to the method described in Example 11. The commercially available superoxide dismutase from bovine erythrocytes and expressed in E. coli of sequence A:

MATKAVCVLKGDGPVQGTIHFEAKGDTVVVTGSITGLTEGDHGFHVHQFG
DNTQGCTSAGPHFNPLSKKHGGPKDEERHVGDLGNVTADKNGVAIVDIVD
PLISLSGEYSIIGRTMVVHEKPDDLGRGGNEESTKTGNAGSRLACGVIGI
AK

retains very little of its activity at 50° C.

Accordingly, one aspect of the invention is directed to an animal feed additive comprising a fungal superoxide dismutase wherein the superoxide dismutase is thermal stable such that it retains at least 40% of its activity when measured at 50° C. according to the test method described in Example 11, such as retaining at least 50% activity, such as retaining at least 55% activity, such as retaining at least 60% activity, such as retaining at least 65% activity, such as retaining at least 70% activity, such as retaining at least 75% activity, such as retaining at least 80% activity.

Thermal Stability Table at different pH - Tm (unfolding temperature)

| SEQ ID | SEQ ID | Tₘ/pH | | | | | | | |
|---|---|---|---|---|---|---|---|---|---|
| NO | NO | 3 | 4 | 5 | 6 | 7 | 8 | 9 | 10 |
| SED ID | SEQ ID | 67.1 | 75.0 | 75.5 | 75.4 | 63.6 | 63.0 | 61.2 | 59.4 |
| NO 1 | NO 83 | | | | | | | | |
| SEQ ID | SEQ ID | na | na | 56.7 | 63.4 | 67.9 | 66.6 | 64.8 | 69.2 |
| NO 9 | NO 49 | | | | | | | | |
| SEQ ID | SEQ ID | 46.6 | 54.3 | 55.5 | 56.9 | 56.7 | 53.4 | 48.9 | 44.1 |
| NO 10 | NO 50 | | | | | | | | |
| SEQ ID | SEQ ID | 44.6 | 56.7 | 61.9 | 61.2 | 56.6 | 54.0 | 50.5 | 45.8 |
| NO 11 | NO 51 | | | | | | | | |
| SEQ ID | SEQ ID | na | 43.0 | 48.0 | 48.8 | 48.1 | 47.0 | 46.2 | 45.3 |
| NO 12 | NO 52 | | | | | | | | |
| SEQ ID | SEQ ID | na | 45.2 | 49.6 | 50.1 | 49.3 | 48.4 | 47.3 | na |
| NO 13 | NO 53 | | | | | | | | |
| SEQ ID | SEQ ID | na | na | na | na | na | 55.5 | 54.4 | 53.2 |
| NO 14 | NO 54 | | | | | | | | |
| SEQ ID | SEQ ID | 52.9 | 63.0 | 64.6 | 59.1 | 53.6 | 51.7 | 50.7 | 49.7 |
| NO 15 | NO 55 | | | | | | | | |
| SEQ ID | SEQ ID | na | 37.6 | 48.7 | 54.3 | 55.0 | 55.6 | 54.6 | 53.7 |
| NO 16 | NO 56 | | | | | | | | |
| SEQ ID | SEQ ID | na | 37.9 | 48.4 | 53.6 | 53.7 | 54.3 | 53.3 | 53.0 |
| NO 17 | NO 18 | | | | | | | | |
| SEQ ID | SEQ ID | na | na | Na | 66.3 | 70.7 | 70.6 | 71.1 | 71.6 |
| NO 19 | NO 57 | | | | | | | | |
| SEQ ID | SEQ ID | na | na | 50.9 | 61.8 | 63.5 | 61.1 | 59.8 | 51.8 |
| NO 21 | NO 59 | | | | | | | | |
| SEQ ID | SEQ ID | 43.7 | 53.3 | 59.3 | 59.3 | 58.1 | 62.1 | 57.7 | 51.1 |
| NO 22 | NO 60 | | | | | | | | |
| SEQ ID | SEQ ID | 43.3 | 51.9 | 58.8 | 59.5 | 57.6 | 53.9 | 52.8 | 50.6 |
| NO 23 | NO 61 | | | | | | | | |
| SEQ ID | SEQ ID | 43.9 | 52.3 | 59.2 | 63.7 | 62.3 | 61.8 | 59.9 | 59.2 |
| NO 24 | NO 62 | | | | | | | | |
| SEQ ID | SEQ ID | 40.6 | 49.2 | 58.1 | 62.8 | 62.1 | 54.7 | 54.2 | 60.8 |
| NO 25 | NO 63 | | | | | | | | |
| SEQ ID | SEQ ID | 50.9 | 64.6 | 68.4 | 73.4 | 74.9 | 74.2 | 73.9 | 72.8 |
| NO 27 | NO 65 | | | | | | | | |
| SEQ ID | SEQ ID | 62.0 | 72.3 | 74.6 | 74.5 | 71.9 | 63.8 | 58.3 | 56.0 |
| NO 28 | NO 66 | | | | | | | | |
| SEQ ID | SEQ ID | 63.7 | 71.0 | 73.0 | 72.9 | 70.6 | 62.5 | 57.2 | 55.7 |
| NO 29 | NO 67 | | | | | | | | |
| SEQ ID | SEQ ID | 62.8 | 71.3 | 73.2 | 73.2 | 71.3 | 63.9 | 57.4 | 55.3 |
| NO 30 | NO 68 | | | | | | | | |
| SEQ ID | SEQ ID | 74.5 | 78.7 | 78.5 | 76.1 | 70.4 | 64.6 | 58.6 | 46.8 |
| NO 31 | NO 69 | | | | | | | | |
| SEQ ID | SEQ ID | 54.3 | 66.7 | 65.2 | 71.6 | 72.5 | 69.7 | 62.2 | 54.6 |
| NO 32 | NO 70 | | | | | | | | |
| SEQ ID | SEQ ID | 54.9 | 63.0 | 68.2 | 68.2 | 66.7 | 64.1 | 62.8 | 61.8 |
| NO 33 | NO 71 | | | | | | | | |
| SEQ ID | SEQ ID | 67.1 | 75.5 | 77.0 | 76.4 | 73.6 | 66.5 | 60.7 | 58.3 |
| NO 34 | NO 35 | | | | | | | | |
| SEQ ID | SEQ ID | 58.8 | 60.9 | 59.9 | 65.2 | 66.5 | 63.6 | 56.3 | 54.4 |
| NO 36 | NO 37 | | | | | | | | |
| SEQ ID | SEQ ID | 60.5 | 71.0 | 72.8 | 72.7 | 71.3 | 60.9 | 57.6 | 55.3 |
| NO 38 | NO 72 | | | | | | | | |
| SEQ ID | SEQ ID | 68.0 | 76.2 | 76.9 | 76.2 | 73.7 | 65.0 | 60.6 | 58.1 |
| NO 39 | NO 73 | | | | | | | | |
| SEQ ID | SEQ ID | 52.8 | 67.6 | 74.7 | 74.2 | 69.2 | 55.9 | 54.1 | 51.9 |
| NO 40 | NO 74 | | | | | | | | |
| SEQ ID | SEQ ID | 53.3 | 68.3 | 75.1 | 77.9 | 75.3 | 64.8 | 59.7 | 56.5 |
| NO 41 | NO 75 | | | | | | | | |
| SEQ ID | SEQ ID | 47.8 | 58.4 | 62.8 | 64.3 | 64.0 | 59.4 | 54.7 | 50.3 |
| NO 42 | NO 76 | | | | | | | | |
| SEQ ID | SEQ ID | 49.5 | 52.9 | 58.5 | 60.3 | 59.0 | 57.0 | 52.8 | 49.8 |
| NO 43 | NO 77 | | | | | | | | |
| SEQ ID | SEQ ID | 51.8 | 67.7 | 74.2 | 76.8 | 77.2 | 83.7 | 77.6 | 65.8 |
| NO 44 | NO 78 | | | | | | | | |
| SEQ ID | SEQ ID | 52.5 | 56.4 | 63.3 | 65.8 | 58.9 | 61.4 | 53.5 | 50.1 |
| NO 45 | NO 79 | | | | | | | | |
| SEQ ID | SEQ ID | 75.3 | 86.5 | >95 | >95 | >95 | 91.1 | 88.9 | 86.8 |
| NO 46 | NO 80 | | | | | | | | |
| SEQ ID | SEQ ID | 46.5 | 57.9 | 62.0 | 61.5 | 57.8 | 56.1 | 52.1 | 51.6 |
| NO 47 | NO 81 | | | | | | | | |
| SEQ ID | SEQ ID | 53.5 | 58.4 | 64.3 | 65.8 | 63.3 | 59.5 | 49.4 | 43.4 |
| NO 48 | NO 82 | | | | | | | | |
| SEQ ID | SEQ ID | 48.9 | na | na | 58.8 | 58.4 | na | 53.5 | 48 |
| NO 199 | NO 210 | | | | | | | | |
| SEQ ID | SEQ ID | na | 61.1 | na | na | 57.1 | 54.9 | na | 47.5 |
| NO 200 | NO 211 | | | | | | | | |
| SEQ ID | SEQ ID | na | na | na | 56.9 | 55.5 | 53.5 | 51.9 | 49.6 |
| NO 201 | NO 212 | | | | | | | | |
| SEQ ID | SEQ ID | 56.7 | 69.4 | 59.9 | 62 | 64.8 | 64.7 | 63.4 | 56.5 |
| NO 202 | NO 213 | | | | | | | | |
| SEQ ID | SEQ ID | 55.3 | na | 63.9 | na | 58.2 | 55.1 | 52.6 | 46 |
| NO 203 | NO 214 | | | | | | | | |
| SEQ ID | SEQ ID | na | na | 59.4 | 60 | na | na | na | 49.2 |
| NO 204 | NO 215 | | | | | | | | |
| SEQ ID | SEQ ID | 57.3 | 66.6 | 68.8 | 68.2 | 64.3 | 57 | 54.1 | 51.8 |
| NO 205 | NO 216 | | | | | | | | |
| SEQ ID | SEQ ID | na | na | na | na | 65.7 | 63.6 | 51.2 | 45.2 |
| NO 206 | NO 217 | | | | | | | | |
| SEQ ID | SEQ ID | na | 72.4 | 81.2 | 84.8 | 86.1 | 86.3 | 86.1 | 85.4 |
| NO 207 | NO 218 | | | | | | | | |
| SEQ ID | SEQ ID | na | 70.5 | 80.1 | 83.1 | 85 | 85.4 | 85.5 | 84.5 |
| NO 208 | NO 219 | | | | | | | | |
| SEQ ID | SEQ ID | na | 72.9 | 81.7 | 84.8 | 86.1 | 86.6 | 86.3 | 85.6 |
| NO 209 | NO 220 | | | | | | | | |

| | | | | | | | | |
|---|---|---|---|---|---|---|---|---|
| Bovine SOD S9697 | 50.8 | 59.3 | 63.9 | 66.2 | 65.2 | 63.1 | 61.0 | 57.0 |
| SIGMA | | | | | | | | |

### Catalases

In a preferred embodiment, the method of the invention, the animal feed additive of the invention and the animal feed of the invention further comprise a polypeptide having catalase activity. The catalase from Bovine Liver (Enzyme Commission (EC) Number: 1.11.1.6, CAS Number: 9001-05-2, Molecular weight: 250 kDa) has an activity of 3524 U/mg EP and a gastric stability wherein it retains only 40% of its activity under the gastric stability studies.

The catalase of the invention is of bacterial origin or of fungal origin, preferably of fungal origin. The catalases of the invention have shown to be either highly active, highly gastric stable or highly thermal stable, or combinations thereof.

### Sequences of Catalases

In a preferred embodiment, the method of the invention, the animal feed additive of the invention and the animal feed of the invention further comprise a polypeptide having catalase activity.

Typically, the polypeptide having catalase activity is selected from the group consisting of:
a. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6; and
b. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7
c. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 103
d. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 104;
e. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 105;
f. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 106;
g. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 107;
h. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 108;
i. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 109
j. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 110
k. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 111
l. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 112
m. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 113
n. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 114
o. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 115
p. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 116
q. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 117
r. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 118
s. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 119
t. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 120
u. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121
v. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122
w. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 123
x. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 124
y. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 125
z. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 126;
aa. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 250;
bb. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 151;
cc. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 152;
dd. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 153;
ee. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 154;
ff. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 155;
gg. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 156;
hh. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 157;
ii. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 158;
jj. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 159;
kk. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 160;
ll. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 161;
mm. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 162;
nn. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 163;
oo. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 164;
pp. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 165;
qq. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 166;
rr. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 167;
ss. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 168;
tt. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 169;
uu. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 170;
vv. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 171;
ww. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 172;
xx. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 173;
yy. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 174;
zz. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 250; and
aaa. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 251.

In a preferred embodiment, the polypeptide having catalase activity is selected from the group consisting of:

a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 6;

a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 7;

a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 250; and

a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 251.

### Sources of Catalases

SEQ ID NO 6 and SEQ ID NO 7 are polypeptide having catalase activity available from Aspergillus niger. SEQ ID NO 7 is sold under the tradename Catazyme™. SEQ ID NO: 103 is a polypeptide having catalase activity available from Thermoascus aurantiacus. SEQ ID NO: 104 is a polypeptide having catalase activity available from Aspergillus lentulus. SEQ ID NO: 105 is a polypeptide having catalase activity available from Talaromyces stipitatus. SEQ ID NO: 106 is a polypeptide having catalase activity available from Malbranchea cinnamomea. SEQ ID NO: 107 is a polypeptide having catalase activity available from Crassicarpon thermophilum. SEQ ID NO: 108 is a polypeptide having catalase activity available from Penicillium emersonii. SEQ ID NO: 109 is a polypeptide having catalase activity available from Aspergillus versicolor. SEQ ID NO: 110 is a polypeptide having catalase activity available from Thermomucor indicae-seudaticae. SEQ ID NO: 111 is a polypeptide having catalase activity available from Aspergillus fumigatus. SEQ ID NO: 112 is a polypeptide having catalase activity available from Thermothelomyces thermophilus. SEQ ID NO: 113 is a polypeptide having catalase activity available from Curvularia verruculosa. SEQ ID NO: 114 is a polypeptide having catalase activity available from Mycothermus thermophilus. SEQ ID NO: 115 is a polypeptide having catalase activity available from Mycothermus thermophilus. SEQ ID NO: 116 is a polypeptide having catalase activity available from Penicillium oxalicum. SEQ ID NO: 117 is a polypeptide having catalase activity available from Humicola hyalothermophila. SEQ ID NO: 118 is a polypeptide having catalase activity available from Thermoascus crustaceus. SEQ ID NO: 119 is a polypeptide having catalase activity available from Thielavia australiensis. SEQ ID NO: 120 is a polypeptide having catalase activity available from Thielavia hyrcaniae. SEQ ID NO: 121 is a polypeptide having catalase activity available from Neurospora crassa. SEQ ID NO: 122 is a polypeptide having catalase activity available from Thermoascus aurantiacus. SEQ ID NO: 123 is a polypeptide having catalase activity available from Neurospora crassa. SEQ ID NO: 124 is a polypeptide having catalase activity available from Thermoascus aurantiacus. SEQ ID NO: 125 is a polypeptide having catalase activity available from Thermoascus aurantiacus. SEQ ID NO: 126 is a polypeptide having catalase activity available from Thermoascus aurantiacus.

Accordingly, the catalase of the invention may be selected from a polypeptide having catalase activity and obtained from, obtainable from, or derivable from a source selected from the group consisting of Aspergillus niger, Thermoascus aurantiacus, Aspergillus lentulus, Scytalidium thermophilum, Talaromyces stipitatus, Malbranchea cinnamomea, Crassicarpon thermophilum, Penicillium emersonii, Aspergillus versicolor, Thermomucor indicae-seudaticae, Aspergillus fumigatus, Thermothelomyces thermophilus, Curvularia verruculosa, Mycothermus thermophilus, Penicillium oxalicum, Humicola hyalothermophila, Thermoascus crustaceus, Thielavia australiensis, Thielavia hyrcaniae and Neurospora crassa.

In a preferred embodiment, the catalase is selected from the group consisting of a catalase obtained from, obtainable from, or derivable from a source selected from the group consisting of Aspergillus niger, Scytalidium thermophilum and Thermoascus aurantiacus, more preferably from Aspergillus niger and Thermoascus aurantiacus

### Activity of Catalases

The catalase from Bovine Liver (Enzyme Commission (EC) Number: 1.11.1.6 CAS Number: 9001-05-2, Molecular weight: 250 kDa) has an activity of 3524 U/mg EP and a gastric stability wherein it retains only 40% of its activity under the gastric stability studies of Example 12.

In a preferred embodiment, the catalase of the invention has an activity level above 5000 U/mg EP when measured according to the method described in Example 11 and/or at pH 7.8. Accordingly, in one embodiment of the invention the catalase is selected from a polypeptide which has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a mature polypeptide selected from the group consisting of SEQ ID NO 250, SEQ ID NO 7, SEQ ID NO 105, SEQ ID NO 108, SEQ ID NO 116, SEQ ID NO 118, SEQ ID NO 112, SEQ ID NO 113, SEQ ID NO 115, SEQ ID NO 104, SEQ ID NO 111, SEQ ID NO 123, SEQ ID NO 121, SEQ ID NO 106, SEQ ID NO 117, SEQ ID NO 119, SEQ ID NO 110, SEQ ID NO 107 or with a full length polypeptide selected from the group consisting of SEQ ID NO 251, SEQ ID NO 8, SEQ ID NO 153, SEQ ID NO 156, SEQ ID NO 164, SEQ ID NO 166, SEQ ID NO 160, SEQ ID NO 161, SEQ ID NO 163, SEQ ID NO 152, SEQ ID NO 159, SEQ ID NO 171, SEQ ID NO 169, SEQ ID NO 154, SEQ ID NO 165, SEQ ID NO 167, SEQ ID NO 158, and SEQ ID NO 155.

| | Mature | Full length | |
|---|---|---|---|
| Source | Polypeptide | polypeptide | Activity |
| Talaromyces stipitatus | SEQ ID NO 105 | SEQ ID NO 153 | 1667 |
| Penicillium emersonii | SEQ ID NO 108 | SEQ ID NO 156 | 8265 |
| Penicillium oxalicum | SEQ ID NO 116 | SEQ ID NO 164 | 30953 |
| Thermoascus crustaceus | SEQ ID NO 118 | SEQ ID NO 166 | 10919 |
| Thermothelomyces thermophilus | SEQ ID NO 112 | SEQ ID NO 160 | 18314 |
| Curvularia verruculosa | SEQ ID NO 113 | SEQ ID NO 161 | 20836 |
| Aspergillus oryzae (NN051131) | SEQ ID NO 115 | SEQ ID NO 163 | 43975 |
| Aspergillus lentulus | SEQ ID NO 104 | SEQ ID NO 152 | 52327 |
| Aspergillus fumigatus | SEQ ID NO 111 | SEQ ID NO 159 | 51648 |
| Neurospora crassa | SEQ ID NO 123 | SEQ ID NO 171 | 8651 |
| Neurospora crassa | SEQ ID NO 121 | SEQ ID NO 169 | 51184 |
| Malbranchea cinnamomea | SEQ ID NO 106 | SEQ ID NO 154 | 82637 |
| Humicola hyalothermophila | SEQ ID NO 117 | SEQ ID NO 165 | 56454 |
| Thielavia australiensis | SEQ ID NO 119 | SEQ ID NO 167 | 5273 |
| Thermomucor indicae-seudaticae | SEQ ID NO 110 | SEQ ID NO 158 | 7750 |
| Crassicarpon thermophilum | SEQ ID NO 107 | SEQ ID NO 155 | 39459 |

### Thermal Stability of Catalases

An aspect of the invention is directed to an animal feed additive comprising a catalase wherein the catalase is thermal stable such that it retains at least 40% of its activity when measured at 50° C. and pH 7 such as retaining at least 50% activity, such as retaining at least 55% activity, such as retaining at least 60% activity, such as retaining at least 65% activity, such as retaining at least 70% activity, such as retaining at least 75% activity, such as retaining at least 80% activity.

An aspect of the invention is directed to an animal feed additive comprising a catalase wherein the catalase is thermal stable such that it retains at least 50% of its activity when measured at 40° C. at pH 7 such as retaining at least 55% activity, such as retaining at least 60% activity, such as retaining at least 65% activity, such as retaining at least 70% activity, such as retaining at least 75% activity, such as retaining at least 80% activity.

Thermal Stability at different pHs

| | Mature | Tₘ | | | | | | | |
|---|---|---|---|---|---|---|---|---|---|
| Source | SEQ ID NO | 3 | 4 | 5 | 6 | 7 | 8 | 9 | 10 |
| Thermoascus | SEQ ID | 58.5 | 70.2 | 80.1 | 82.1 | 84.0 | 83.8 | 77.9 | 79.9 |
| aurantiacus | NO 250 | | | | | | | | |
| Aspergillus | SEQ ID | 56.0 | 68.2 | 72.2 | 72.3 | 70.2 | 67.0 | 65.7 | 66.1 |
| niger | NO 7 | | | | | | | | |
| Thermoascus | SEQ ID | 67.1 | 75.7 | 79.9 | 80.7 | 79.7 | 79.9 | 78.8 | 71.0 |
| aurantiacus | NO 251 | | | | | | | | |
| Talaromyces | SEQ ID | na | na | na | na | 41.8 | 41.9 | 42.6 | 42.6 |
| stipitatus | NO 105 | | | | | | | | |
| Penicillium | SEQ ID | na | na | na | 45.3 | 50.4 | 50.4 | 50.2 | 50.0 |
| emersonii | NO 108 | | | | | | | | |
| Penicillium | SEQ ID | na | 51.9 | 59.6 | 63.6 | 73.8 | 65.7 | 63.1 | 57.1 |
| oxalicum | NO 116 | | | | | | | | |
| Thermoascus | SEQ ID | 52.8 | 57.7 | 59.7 | 59.2 | 55.4 | 53.7 | 53.1 | 52.3 |
| crustaceus | NO 118 | | | | | | | | |
| Thermothelomyces | SEQ ID | na | na | na | na | na | 60.1 | 60.6 | na |
| thermophilus | NO 112 | | | | | | | | |
| Scytalidium | Note A | 78.3 | >95 | >95 | >95 | >95 | 89.8 | 86.0 | 78.7 |
| thermophilum | | | | | | | | | |
| Curvularia | SEQ ID | 44.8 | 57.3 | 60.4 | 59.8 | 57.8 | 57 | 54.2 | 49.1 |
| verruculosa | NO 113 | | | | | | | | |
| Aspergillus | SEQ ID | na | na | 60.3 | 61.7 | 63 | na | 62.1 | 55.3 |
| oryzae | NO 115 | | | | | | | | |
| Aspergillus | SEQ ID | na | 66.5 | na | na | 85.1 | na | 72.6 | 73.4 |
| lentulus | NO 104 | | | | | | | | |
| Aspergillus | SEQ ID | na | na | na | na | 83.7 | 82.1 | 78.6 | 73.1 |
| fumigatus | NO 111 | | | | | | | | |
| Neurospora | SEQ ID | na | na | na | na | 67.6 | 61.5 | 61.7 | na |
| crassa | NO 123 | | | | | | | | |
| Neurospora | SEQ ID | na | na | na | 9.5 | 81.8 | 81.2 | 80.5 | na |
| crassa | NO 121 | | | | | | | | |
| Malbranchea | SEQ ID | na | na | 69.7 | 77.3 | 84.2 | 85 | 85.2 | na |
| cinnamomea | NO 106 | | | | | | | | |
| Humicola | SEQ ID | na | na | 6.8 | na | na | na | 89.2 | 83.6 |
| hyalothermophila | NO 117 | | | | | | | | |
| Thielavia | SEQ ID | na | na | na | 47.4 | 64.3 | 57 | 52.1 | 45.2 |
| australiensis | NO 119 | | | | | | | | |
| Crassicarpon | SEQ ID | na | na | na | na | na | 85.3 | 83.3 | 73.4 |
| thermophilum | NO 107 | | | | | | | | |

Note A:
SEQ ID NO: 2 of U.S. Pat. No. 5,646,025

An aspect of the invention is directed to an animal feed additive comprising a catalase wherein the catalase is thermal stable such that it retains at least 50% of its activity when measured at 50° C. and pH 5 such as retaining at least 55% activity, such as retaining at least 60% activity, such as retaining least 65% activity, such as retaining at least 70% activity, such as retaining at least 75% activity, such as retaining at least 80% activity. An aspect of the invention is directed to an animal feed additive comprising a catalase wherein the catalase is thermal stable such that its Tm is at least 50° C. at pH 5.

An aspect of the invention is directed to an animal feed additive comprising a catalase wherein the catalase is thermal stable such that it retains at least 50% of its activity when measured at 50° C. and pH 4 such as retaining at least 55% activity, such as retaining at least 60% activity, such as retaining least 65% activity, such as retaining at least 70% activity, such as retaining at least 75% activity, such as retaining at least 80% activity. An aspect of the invention is directed to an animal feed additive comprising a catalase wherein the catalase is thermal stable such that its Tm is at least 50° C. at pH 4.

An aspect of the invention is directed to an animal feed additive comprising a catalase wherein the catalase is thermal stable such that it retains at least 50% of its activity when measured at 40° C. and pH 3 such as retaining at least 55% activity, such as retaining at least 60% activity, such as retaining least 65% activity, such as retaining at least 70% activity, such as retaining at least 75% activity, such as retaining at least 80% activity. An aspect of the invention is directed to an animal feed additive comprising a catalase wherein the catalase is thermal stable such that its Tm is at least 40° C. at pH 3.

### Gastric Stability of Catalases

The catalase from Bovine Liver (Enzyme Commission (EC) Number: 1.11.1.6 CAS Number: 9001-05-2, Molecular weight: 250 kDa) has an activity of 3524 U/mg EP and a gastric stability wherein it retains only 40% of its activity under the gastric stability studies of Example 12.

One aspect of the invention is directed to an animal feed additive further comprising a catalase wherein the catalase is gastric stable such that it retains at least 40% of its activity when measured according to the test method described in Example 12, such as retaining at least 50% activity, such as retaining at least 55% activity, such as retaining at least 60% activity, such as retaining at least 65% activity, such as retaining at least 70% activity, such as retaining at least 75% activity, such as retaining at least 80% activity. As can be seen from the Table in Example 12, each of SEQ ID NO 109, SEQ ID NO 126, SEQ ID NO 7, (SEQ ID NO 250), SEQ ID NO 105, SEQ ID NO 108, SEQ ID NO 116, SEQ ID NO 105, SEQ ID NO 112, SEQ ID NO 115, SEQ ID NO 104, SEQ ID NO 117, SEQ ID NO 107, SEQ ID NO 110, SEQ ID NO 123 retain at least 50% of their activity at pH 3 and exposure to pepsin.

### Animal Feed Additive and Animal Feed

The invention is directed to an animal feed additive and to an animal feed comprising the animal feed additive as defined herein. An aspect of the invention is directed to an animal feed additive comprising a polypeptide of fungal origin having superoxide dismutase activity. The animal feed additive may further comprising a catalase. The animal feed additive may alternatively be defined as comprising an enzyme component, wherein the enzyme component comprises all of the enzymes of the additive and the enzyme component consists of a superoxide dismutase of fungal origin and optionally of a catalase. In a further aspect, the invention is directed to an animal feed additive comprising at least one and no more than two enzyme classes, wherein the at least one enzyme class is a superoxide dismutase of fungal origin and the optional second enzyme class is a catalase.

In an embodiment, the animal feed additive is wherein the polypeptide having superoxide dismutase activity is selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

In an embodiment, the animal feed additive is wherein the polypeptide having superoxide dismutase activity is selected from the group consisting of:
a. a polypeptide Trichoderma reesei from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
m. a polypeptide from Cladobotryum sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
ee. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
uu. a polypeptide Mariannaea punicea from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.

In an embodiment, the animal feed additive is wherein the polypeptide having superoxide dismutase activity comprises an amino acid sequence selected from the group consisting of:
a. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1;
b. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2;
c. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3;
d. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4;
e. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9;
f. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10;
g. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11;
h. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 12;
i. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 13;
j. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 14;
k. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 15;
l. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 16;
m. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17;
n. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19;
o. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 20;
p. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21;
q. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22;
r. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23;
s. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 24;
t. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 sequence identity to the amino acid sequence of SEQ ID NO: 25;
u. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26;
v. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 27;
w. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 28;
x. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 29;
y. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 30;
z. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 31;
aa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 32;
bb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33;
cc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 34;
dd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 36;
ee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 38;
ff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 39;
gg. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
hh. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
ii. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 42;
jj. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43;
kk. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44;
ll. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 45;
mm. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 46;
nn. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 47; and
oo. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 48;
pp. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:199;
qq. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:200;
rr. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO 201;
ss. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:202;
tt. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:203;
uu. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:204;
vv. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 205;
ww. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:206;
xx. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:207;
yy. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:208;
zz. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:209;
aaa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:232;
bbb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:233;
ccc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:234;
ddd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:235;
eee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:236; and
fff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:237.

In an embodiment, the animal feed additive is wherein the polypeptide having superoxide dismutase activity is selected from the group consisting
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

In an embodiment, the animal feed additive is wherein the polypeptide having superoxide dismutase activity is selected from the group consisting of
a. a polypeptide from Trichoderma reesei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
m. a polypeptide from Cladobotryum sp having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
ee. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
uu. a polypeptide from Mariannaea punicea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.

The animal feed additive suitably comprises one or more polypeptides of fungal origin having superoxide dismutase (SOD) activity wherein the polypeptide having superoxide dismutase activity is selected from the group consisting of:
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.

In a further embodiment, the invention relates to an animal feed additive or animal feed premix comprising one or more polypeptides having superoxide dismutase (SOD), wherein the feed additive or premix further comprises
a. one or more polypeptides having catalase activity and/or
b. one or more vitamins, wherein the one or more vitamins is preferably a fat-soluble vitamin, for example vitamin E.

In a preferred embodiment, a catalase is further added to the animal feed additive or animal feed or animal feed premix comprising the fungal SOD. A catalase may be classified as an EC 1.11.1.6 catalase or as an EP 1.11.1.21 catalase peroxidase. A preferred example of the catalase according to the invention is a polypeptide having at least 80% sequence identity to SEQ ID NO:6 and SEQ ID NO:7.

A preferred animal feed premix, animal feed additive, or animal feed comprises one or more polypeptides having superoxide dismutase (SOD), vitamin E and optionally selenium and is used as antioxidant, preferably in feed and feed premixes or as a replacement or partial replacement of antibiotics in animal feed.

Examples of commercial vitamin E and selenium are Rovimix®E50 and SePlex (DSM Nutritional Products).

In a further embodiment, the animal feed or animal feed additive further comprises one or more components selected from the list consisting of:
i. one or more carriers;
ii. one or more microbes;
iii. one or more vitamins;
iv. one or more minerals;
v. one or more amino acids;
vi. one of more organic acids;
vii. and one or more other feed ingredients.

The animal feed or animal feed additive may furthermore comprise additional enzymes but enzymes other than fungal superoxide dismutase and optionally a catalase, are not essential for the beneficial effects of the invention. The feed additive typically comprises one or more fungal superoxide dismutases, one or more polypeptides having catalase activity and/or further comprising one or more vitamins.

In one aspect, the invention relates to an animal feed comprising an animal feed additive, one or more protein sources and one or more energy sources characterised in the animal feed further comprises one or more polypeptides having SOD activity, wherein the animal feed
a. reduces FCR by between 1 to 10%; or
b. increases average daily WG by between 1 to 10%; or
c. increases average daily feed intake by between 1 to 10%; or
d. any combinations of a.-c.;
when feed to the animal as compared to animals fed a feed composition without the polypeptides.

In a further aspect, the invention relates to an animal feed comprising an animal feed additive, one or more protein sources and one or more energy sources characterised in the animal feed further comprises one or more polypeptides having SOD activity, wherein the animal feed
a. improves or enhances immune response; or
b. reduces inflammation;
when feed to the animal as compared to animals fed a feed composition without the polypeptides.

A further aspect of the invention is directed to the prophylactic care or management, reduction or prevention of oxidative stress in a monogastric animal comprising administrating to said animal superoxide dismutase and optionally catalase. Oxidative stress is a disturbance between antioxidant/oxidant status in favor of excessive generation, or slower removal of free radicals, such as reactive oxygen species (ROS). Excessive ROS content leads to damage of proteins, lipids and nucleic acids, with consequent loss of their biological functions and subsequent tissue injury. Oxidative stress has been linked to initiation and progression of several infectious diseases. Accordingly, a further aspect of the invention is the prophylactic care or management of infectious diseases in monogastric animal comprising administrating to said animal superoxide dismutase and optionally catalase. The administration is typically by means of feeding said animal a feed additive comprising an enzyme component, wherein the enzyme component comprises all of the enzymes of the additive and consists of a superoxide dismutase and optionally of a catalase.

In respect to the improvement of one or more performance parameters, the inventions is particularly characterized in that the EPEF and/or FCR and/or GR and/or WG is improved by at least 1% and that the MR is reduced by at least 1%.

The animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pets (including but not limited to cats and dogs); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick, typically wherein the animal has experienced heat stress, cold stress, nutritional stress and/or oxidative stress.

The polypeptide having catalase activity is preferably dosed at a level of 100 to 1000 U enzyme protein per kg animal feed, such as 200 to 900 U, 300 to 800, 400 to 700, 500 to 600 enzyme protein per kg animal feed, or any combination of these intervals.

The polypeptide having superoxide dismutase activity is preferably dosed at a level of 100 to 5000 U enzyme protein per kg animal feed, such as 200 to 3000 U, 500 to 2500 U, 500 to 2000 U, 500 to 1500 U enzyme protein per kg animal feed, or any combination of these intervals.

Examples of dosing include SEQ ID No. 1 at 1250 or 2500 U/kg, SEQ ID NO 7 and SEQ ID NO 250 at 100 U/kg) and combinations (SEQ ID No. 1: 500 U/kg+SEQ ID NO 7: 100 U/kg; and SEQ ID No. 1: 500 U/kg+SEQ ID NO 250: 100 U/kg.

The protein source of the animal feed is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

The energy source of the animal feed is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

In a preferred example, the animal feed further comprises one or more components selected from the list consisting of one or more additional enzymes; one or more microbes; one or more vitamins; one or more minerals; one or more amino acids; and one or more other feed ingredients, as described herein.

### Methods of the Invention

In a further aspect, the invention relates to a method of improving one or more performance parameters in an animal comprising administering to the animal an animal feed or animal feed additive comprising one or more polypeptides having superoxide dismutase (SOD) activity of fungal origin, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).

In a further aspect, the invention relates to a method of improving or enhancing immune response and/or reducing inflammation and/or for the modulation of the gut flora in an animal comprising administering to the animal an animal feed or animal feed additive comprising one or more polypeptides having superoxide dismutase activity. A related aspect of the invention is directed to the prophylactic care or management, reduction or prevention of inflammation in the intestinal tract of a monogastric animal.

In a further aspect, the invention relates to a method of reducing or eliminating the use of antibiotics administered to animal feed, comprising administering to the animal an animal feed or animal feed additive comprising of one or more polypeptides having superoxide dismutase activity.

In a further aspect, the invention relates to a method of reducing cellular markers of reactive oxygen species or free radicals in animal body comprising administering to the animal an animal feed or animal feed additive comprising one or more polypeptides having superoxide dismutase (SOD) activity.

A further aspect of the invention is directed to the prophylactic care or management, reduction or prevention of oxidative stress in a monogastric animal comprising administrating to said animal superoxide dismutase and optionally catalase. Oxidative stress is a disturbance between antioxidant/oxidant status in favor of excessive generation, or slower removal of free radicals, such as reactive oxygen species (ROS). Excessive ROS content leads to damage of proteins, lipids and nucleic acids, with consequent loss of their biological functions and subsequent tissue injury. Oxidative stress has been linked to initiation and progression of several infectious diseases. Accordingly, a further aspect of the invention is the prophylactic care or management of infectious diseases in monogastric animal comprising administrating to said animal superoxide dismutase and optionally catalase. The administration is typically by means of feeding said animal a feed additive comprising an enzyme component, wherein the enzyme component comprises all of the enzymes of the additive and consists of a superoxide dismutase and optionally of a catalase.

In respect to the improvement of one or more performance parameters, the inventions is particularly characterized in that the EPEF and/or FCR and/or GR and/or WG is improved by at least 1% and that the MR is reduced by at least 1%.

The animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pets (including but not limited to cats and dogs); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick, typically wherein the animal has experienced heat stress, cold stress, nutritional stress and/or oxidative stress.

A further aspect of the invention is directed to a method of feeding poultry or pigs comprising adding the animal feed additive of the invention to a raw feed material.

A further aspect of the invention is directed to a method of feeding an animal, wherein the animal feed or animal feed additive further comprises one or more components selected from the list consisting of:

viii. one or more carriers;

ix. one or more microbes;

x. one or more vitamins;

xi. one or more minerals;

xii. one or more amino acids;

xiii. one of more organic acids;

xiv. and one or more other feed ingredients.

One may furthermore administer additional enzymes but enzymes other than fungal superoxide dismutase and optionally a catalase, are not essential for the beneficial effects of the invention.

### Enzyme Compositions and Formulations

The polypeptide having SOD activity of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus, in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

In one embodiment, the liquid formulation further comprises 20%-80% polyol (i.e. total amount of polyol), preferably 25%-75% polyol, more preferably 30%-70% polyol, more preferably 35%-65% polyol or most preferably 40%-60% polyol. In one embodiment, the liquid formulation comprises 20%-80% polyol, preferably 25%-75% polyol, more preferably 30%-70% polyol, more preferably 35%-65% polyol or most preferably 40%-60% polyol wherein the polyol is selected from the group consisting of glycerol, sorbitol, propylene glycol (MPG), ethylene glycol, diethylene glycol, triethylene glycol, 1, 2-propylene glycol or 1, 3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e. total amount of polyol), preferably 25%-75% polyol, more preferably 30%-70% polyol, more preferably 35%-65% polyol or most preferably 40%-60% polyol wherein the polyol is selected from the group consisting of glycerol, sorbitol and propylene glycol (MPG).

In one embodiment, the liquid formulation further comprises preservative, preferably selected from the group consisting of sodium sorbate, potassium sorbate, sodium benzoate and potassium benzoate or any combination thereof. In one embodiment, the liquid formulation comprises 0.02% to 1.5% w/w preservative, more preferably 0.05% to 1.0% w/w preservative or most preferably 0.1% to 0.5% w/w preservative. In one embodiment, the liquid formulation comprises 0.001% to 2.0% w/w preservative (i.e. total amount of preservative), preferably 0.02% to 1.5% w/w preservative, more preferably 0.05% to 1.0% w/w preservative or most preferably 0.1% to 0.5% w/w preservative wherein the preservative is selected from the group consisting of sodium sorbate, potassium sorbate, sodium benzoate and potassium benzoate or any combination thereof.

For a solid formulation, the formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In one embodiment, the composition is a solid composition, such as a spray dried composition, comprising the polypeptide having SOD activity of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate, magnesium sulfate and calcium carbonate.

The present invention also relates to enzyme granules/particles comprising the polypeptide having SOD activity of the invention optionally combined with one or more additional enzymes. The granule is composed of a core, and optionally one or more coatings (outer layers) surrounding the core.

Typically, the granule/particle size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known feed and granule formulation technologies, e.g.:

a) spray dried products, wherein a liquid enzyme-containing solution is atomized in a spray drying tower to form small droplets which during their way down the drying tower dry to form an enzyme-containing particulate material;

b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;

c) absorbed core particles, wherein rather than coating the enzyme as a layer around the core, the enzyme is absorbed onto and/or into the surface of the core. Such a process is described in WO 97/39116.

d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;

e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique;

f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.

g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);

h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;

i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In one embodiment, the core comprises a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In one embodiment, the core comprises a clay mineral such as kaolinite or kaolin.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In some embodiments the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

The granule may comprise a core comprising the polypeptide having SOD activity of the invention, one or more salt coatings and one or more wax coatings. Examples of enzyme granules with multiple coatings are shown in WO1993/07263, WO1997/23606 and WO2016/149636.

A salt coating may comprise at least 60% by weight of a salt, e.g., at least 65%, at least 70%, at least 75%, at least 80%, at least 85%, at least 90%, at least 95% or at least 99% by weight.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4)2HPO4 (CH20° C.=93%), NH4H2PO4 (CH20° C.=93.1%), (NH4)2SO4 (CH20° C.=81.1%), KCl (CH20° C.=85%), K2HPO4 (CH20° C.=92%), KH2PO4 (CH20° C.=96.5%), KNO3 (CH20° C.=93.5%), Na2SO4 (CH20° C.=93%), K2SO4 (CH20° C.=98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4)H2PO4, CuSO4, Mg(NO3)2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO4), magnesium sulfate heptahydrate (MgSO4.7H2O), zinc sulfate heptahydrate (ZnSO4.7H2O), sodium phosphate dibasic heptahydrate (Na2HPO4.7H2O), magnesium nitrate hexahydrate (Mg(NO3)2(6H2O)), sodium citrate dihydrate and magnesium acetate tetrahydrate.

Preferably the salt is applied as a solution of the salt, e.g., using a fluid bed.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

Non-dusting granulates may be produced, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

### Animal Feed

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore, such fish diets usually have a crude fat content of 200-310 g/kg.

An animal feed composition according to the invention has a crude protein content of 50-800 g/kg, and furthermore comprises one or more polypeptides having SOD activity as described herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) SOD/enzyme preparation may also be added before or during the feed ingredient step. Typically a liquid enzyme preparation comprises the SOD of the invention optionally with a polyol, such as glycerol, ethylene glycol or propylene glycol, and is added after the pelleting step, such as by spraying the liquid formulation onto the pellets. The SOD may also be incorporated in a feed additive or premix.

In an embodiment, the composition comprises one or more additional enzymes. In an embodiment, the composition comprises one or more microbes. In an embodiment, the composition comprises one or more vitamins. In an embodiment, the composition comprises one or more minerals. In an embodiment, the composition comprises one or more amino acids. In an embodiment, the composition comprises one or more other feed ingredients.

In another embodiment, the composition comprises one or more of the polypeptides of the invention, one or more formulating agents and one or more additional enzymes. In an embodiment, the composition comprises one or more of the polypeptides of the invention, one or more formulating agents and one or more microbes. In an embodiment, the composition comprises one or more of the polypeptides of the invention, one or more formulating agents and one or more vitamins. In an embodiment, the composition comprises one or more of the polypeptides of the invention and one or more minerals. In an embodiment, the composition comprises the polypeptide of the invention, one or more formulating agents and one or more amino acids. In an embodiment, the composition comprises one or more of the polypeptides of the invention, one or more formulating agents and one or more other feed ingredients.

In a further embodiment, the composition comprises one or more of the polypeptides of the invention, one or more formulating agents and one or more components selected from the list consisting of: one or more additional enzymes; one or more microbes; one or more vitamins; one or more minerals; one or more amino acids; and one or more other feed ingredients.

The final SOD concentration in the diet is within the range of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

The final SOD concentration in the diet can also be determined in Units/kg feed, which is within the range of 100 to 3000 Units per kg animal feed, such as 200 to 3000 U/kg, 300 to 2000 U/kg, 100 to 800 U/kg, 100 to 400 U/kg, or any combination of these intervals.

In another embodiment, the compositions described herein optionally include one or more enzymes for improving feed digestibility. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Thus the composition of the invention may also comprise at least one other enzyme selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), pullulanase (EC 3.2.1.41), pectinesterase (EC 3.1.1.11), beta-xylosidase (EC 3.2.1.37), or any combination thereof.

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

In one embodiment, the amount of vitamins is 0.001% to 10% by weight of the composition. In one embodiment, the amount of minerals is 0.001% to 10% by weight of the composition.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

In a still further embodiment, the animal feed additive of the invention comprises at least one of the below vitamins, preferably to provide an in-feed-concentration within the ranges specified in the below Table 1 (for piglet diets, and broiler diets, respectively).

In the suitable embodiments, the invention relates to an animal feed and a method of improving one or more performance parameters in an animal comprising administering to the animal an animal feed or animal feed additive comprising one or more polypeptides having superoxide dismutase (SOD) activity, wherein the one or more performance parameters is selected from the group consisting of the European Production Efficiency Factor (EPEF), Feed Conversion Ratio (FCR), Growth Rate (GR), Body Weight Gain (WG), Mortility Rate (MR) and Flock Uniformity (FU).

These features are supported by examples 1 and 2. As it is generally known, an improved FCR is lower than the control FCR. In particular embodiments, the FCR is improved (i.e., reduced) as compared to the control by at least 1.0%, preferably at least 1.5%, 1.6%, 1.7%, 1.8%, 1.9%, 2.0%, 2.1%, 2.2%, 2.3%, 2.4%, or at least 2.5%.

The term “mortality” as used herein refers to the ratio of life animals at the end of the growth phase versus the number of animals originally included into the pond. It may be determined on the basis of a fish challenge trial comprising two groups of fish challenged by a particular fish pathogen with the aim to provoke a mortality of 40 to 80% of the animals in the untreated group. However, in the challenge group fed with a suitable concentration per Kg of feed of a mixture of at least two compounds according to the invention, the mortality is reduced compared to the untreated group by at least 5%, preferably at least, 10%, 15%, 20%, 25%, 30%, 35%, 40%, 45%, or at least 50%.

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

In some embodiments the invention relates to an animal feed and a method of improving or enhancing immune response and/or reducing inflammation and/or for the modulation of the gut flora in an animal comprising administering to the animal an animal feed or animal feed additive comprising one or more polypeptides having superoxide dismutase activity.

These features are supported by example 1 as the first two features are very much linked to oxidative stress. Different in-vitro models tested by the applicant also show that SODs optionally in combinations with a catalase are very effective to decrease oxidative stress/burst.

Dysregulating effects of heat stress and oxidative stress also help in maintaining gut integrity and function. Therefore, the invention also supports a positive modulation of the gut flora, in particular of the microbial gut flora.

The term “gut” as used herein designates the gastrointestinal or digestive tract (also referred to as the alimentary canal) and it refers to the system of organs within multi-cellular animals which takes in food, digests it to extract energy and nutrients, and expels the remaining waste.

The term gut “microflora” as used herein refers to the natural microbial cultures residing in the gut and maintaining health by aiding in proper digestion.

The term “modulate” as used herein in connection with the gut microflora generally means to change, manipulate, alter, or adjust the function or status thereof in a healthy and normally functioning animal, i.e. a non-therapeutic use.

The term “supporting immune system function” as used herein refers to the immune stimulation effect obtained by the compounds according to the invention.

In the fifth and sixth embodiment, the invention relates to a method of reducing or eliminating the use of antibiotics administered to animal feed or to a method of reducing cellular markers of reactive oxygen species or free radicals in animal body comprising administering to the animal an animal feed or animal feed additive comprising one or more polypeptides having superoxide dismutase (SOD) activity. These embodiments are supported by example 1.

In the seventh embodiment, the invention relates to an animal feed additive or animal feed premix comprising one or more polypeptides having superoxide dismutase (SOD), wherein the feed additive or premix further comprises
one or more polypeptides having catalase activity and/or
one or more vitamins, wherein the one or more vitamins is preferably a fat-soluble vitamin, for example vitamin E.

If a catalase (CAT) is added to the final feed, the final CAT concentration in the diet is within the range of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

The final CAT concentration in the diet can also be determined in Units/kg feed, which is within the range of 50 to 1000 Units per kg animal feed, such as 50 to 800 U/kg, 100 to 800 U/kg, 100 to 500 U/kg or any combination of these intervals.

As shown in example 1, such a premix has strong antioxidative properties and can be used, optionally in combination with selenium as an antioxidant in feed and feed premixes or as a replacement or partial replacement of antibiotics in animal feed.

### The Invention Comprising the Following Aspects and Embodiments:

1. An isolated polypeptide having superoxide dismutase activity selected from the group consisting of:
   a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
   c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
   d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
   e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
   f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
   g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
   h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
   i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
   j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
   k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
   l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
   m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
   n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
   o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
   p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
   q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
   r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
   s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
   t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
   u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
   v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
   w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
   x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
   y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
   z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
   aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
   bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
   cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
   dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
   ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
   ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
   gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
   hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
   ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
   jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
   kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
   ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
   mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
   nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
   oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
   pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
   qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
   rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
   ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
   tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
   uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
   vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
   ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
   xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
   yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
   zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
   aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
   bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
   ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
   ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
   eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
   fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.
2. An isolated polypeptide having superoxide dismutase activity comprising an amino acid sequence selected from the group consisting of:
   a. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1;
   b. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2;
   c. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3;
   d. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4;
   e. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9;
   f. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10;
   g. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11;
   h. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 12;
   i. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 13;
   j. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 14;
   k. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 15;
   l. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 16;
   m. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17;
   n. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19;
   o. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 20;
   p. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21;
   q. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22;
   r. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23;
   s. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 24;
   t. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 sequence identity to the amino acid sequence of SEQ ID NO: 25;
   u. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26;
   v. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 27;
   w. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 28;
   x. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 29;
   y. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 30;
   z. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 31;
   aa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 32;
   bb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33;
   cc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 34;
   dd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 36;
   ee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 38;
   ff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 39;
   gg. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
   hh. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
   ii. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 42;
   jj. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43;
   kk. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44;
   ll. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 45;
   mm. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 46;
   nn. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 47; and
   oo. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 48;
   pp. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:199;
   qq. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:200;
   rr. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO 201;
   ss. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:202;
   tt. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:203;
   uu. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:204;
   vv. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 205;
   ww. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:206;
   xx. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:207;
   yy. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:208;
   zz. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:209;
   aaa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:232;
   bbb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:233;
   ccc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:234;
   ddd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:235;
   eee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:236; and
   fff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:237.
3. An isolated polypeptide having superoxide dismutase activity selected from the group consisting of
   a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
   b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
   c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
   d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
   e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
   f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
   g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
   h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
   i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
   j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
   k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
   l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
   m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
   n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
   o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
   p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
   q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
   r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
   s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
   t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
   u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
   v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
   w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
   x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
   y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
   z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
   aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
   bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
   cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
   dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
   ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
   ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
   gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
   hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
   ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
   jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
   kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
   ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
   mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
   nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
   oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
   pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
   qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
   rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
   ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
   tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
   uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
   vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
   ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
   xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
   yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
   zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
   aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
   bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
   ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
   ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
   eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
   fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.
4. An isolated polypeptide of having superoxide dismutase activity selected from the group consisting of
   a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 307 of SEQ ID NO 83;
   b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 206 of SEQ ID NO 84;
   c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 154 of SEQ ID NO 85;
   d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 188 of SEQ ID NO 86;
   e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 49;
   f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 184 of SEQ ID NO 50;
   g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 249 of SEQ ID NO 51;
   h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 178 of SEQ ID NO 52;
   i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 248 of SEQ ID NO 53;
   j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 54;
   k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 274 of SEQ ID NO 55;
   l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 26 to 257 of SEQ ID NO 56;
   m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 261 of SEQ ID NO 18;
   n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 247 of SEQ ID NO 57;
   o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 269 of SEQ ID NO 58;
   p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 151 of SEQ ID NO 59,
   q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 228 of SEQ ID NO 60,
   r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 37 to 233 of SEQ ID NO 61;
   s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 33 to 227 of SEQ ID NO 62;
   t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 34 to 230 of SEQ ID NO 63;
   u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 233 of SEQ ID NO 64;
   v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 25 to 227 of SEQ ID NO 65;
   w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 211 of SEQ ID NO 66;
   x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 218 of SEQ ID NO 67;
   y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 218 of SEQ ID NO 68;
   z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 17 to 182 of SEQ ID NO 69;
   aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 185 of SEQ ID NO 70;
   bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 185 of SEQ ID NO 71;
   cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 35;
   dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 30 μL of SEQ ID NO 37;
   ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 310 of SEQ ID NO 72;
   ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 303 of SEQ ID NO 73;
   gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 74;
   hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 75;
   ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 209 of SEQ ID NO 76;
   jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 209 of SEQ ID NO 77;
   kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 22 to 183 of SEQ ID NO 78;
   ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 193 of SEQ ID NO 79;
   mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 177 of SEQ ID NO 80;
   nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 280 of SEQ ID NO 81;
   oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 23 to 215 of SEQ ID NO 82;
   pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 210;
   qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 181 of SEQ ID NO 211;
   rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 182 of SEQ ID NO 212;
   ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 20 to 154 of SEQ ID NO 213;
   tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 214;
   uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 154 of SEQ ID NO 215;
   vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 1 to 150 of SEQ ID NO 216;
   ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 247 of SEQ ID NO 217;
   xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 154 of SEQ ID NO 218;
   yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 316 of SEQ ID NO 219;
   zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 21 to 193 of SEQ ID NO 220;
   aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 186 of SEQ ID NO 238;
   bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 14 to 164 of SEQ ID NO 239;
   ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 184 of SEQ ID NO 240;
   ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 242 of SEQ ID NO 241;
   eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 18 to 261 of SEQ ID NO 242; and
   fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 19 to 258 of SEQ ID NO 243.
5. An isolated polypeptide of having superoxide dismutase activity selected from the group consisting of
   a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
   b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
   c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
   d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
   e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
   f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
   g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
   h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
   i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
   j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
   k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
   l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
   m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
   n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
   o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
   p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
   q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
   r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
   s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
   t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
   u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
   v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
   w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
   x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
   y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
   z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
   aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
   bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
   cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
   dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
   ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
   ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
   gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
   hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
   ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
   jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
   kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
   ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
   mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
   nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
   oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
   pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
   qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
   rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
   ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
   tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
   uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
   vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
   ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
   xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
   yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
   zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
   aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
   bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
   ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
   ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
   eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
   fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.
6. The polypeptide as defined in any of embodiments 1 to 5 of fungal origin.
7. An animal feed additive comprising a polypeptide of fungal origin having superoxide dismutase activity.
8. The animal feed additive according to claim 1 further comprising a catalase.
9. The animal feed additive according to claim 1 comprising an enzyme component, wherein the enzyme component comprises all of the enzymes of the additive and the enzyme component consists of a superoxide dismutase of fungal origin and optionally of a catalase.
10. An animal feed additive comprising at least one and no more than two enzyme classes, wherein the at least one enzyme class is a superoxide dismutase of fungal origin and the optional second enzyme class is a catalase.
11. The animal feed additive according to any of embodiments 7 to 10 wherein the polypeptide having superoxide dismutase activity is as defined in any of embodiments 1 to 5.
12. The animal feed additive according to any of embodiments 7 to 10 wherein the polypeptide having superoxide dismutase activity is selected from the group consisting of
   a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
   c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
   d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
   e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
   f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
   g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
   h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
   i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
   j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
   k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
   l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
   m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
   n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
   o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
   p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
   q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
   r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
   s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
   t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
   u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
   v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
   w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
   x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
   y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
   z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
   aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
   bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
   cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
   dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
   ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
   ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
   gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
   hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
   ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
   jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
   kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
   ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
   mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
   nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
   oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
   pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
   qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
   rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
   ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
   tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
   uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
   vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
   ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
   xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
   yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
   zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
   aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
   bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
   ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
   ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
   eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
   fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.
13. The animal feed additive according to any of embodiments 7 to 10 wherein the polypeptide having superoxide dismutase activity is selected from the group consisting of:
   a. a polypeptide Trichoderma reesei from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
   c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
   d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
   e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
   f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10;
   g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
   h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
   i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
   j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
   k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
   l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
   m. a polypeptide from Cladobotryum sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
   n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19;
   o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20;
   p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21;
   q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22;
   r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23;
   s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
   t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 25;
   u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 26;
   v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27;
   w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28;
   x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29;
   y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30;
   z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31;
   aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32;
   bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33;
   cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34;
   dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36;
   ee. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38;
   ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39;
   gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
   hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
   ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42;
   jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43;
   kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
   ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
   mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46;
   nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; and
   oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
   pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:199;
   qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:200;
   rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 201;
   ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:202;
   tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:203;
   uu. a polypeptide Mariannaea punicea from having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:204;
   vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 205;
   ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:206;
   xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:207;
   yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:208;
   zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:209;
   aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:232;
   bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:233;
   ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:234;
   ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:235;
   eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:236; and
   fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:237.
14. The animal feed additive according to any of embodiments 7 to 10 wherein the polypeptide having superoxide dismutase activity comprises an amino acid sequence selected from the group consisting of:
   a. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 1;
   b. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 2;
   c. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3;
   d. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4;
   e. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9;
   f. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10;
   g. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 11;
   h. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 12;
   i. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 13;
   j. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 14;
   k. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 15;
   l. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 16;
   m. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17;
   n. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19;
   o. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 20;
   p. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21;
   q. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22;
   r. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23;
   s. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 24;
   t. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 sequence identity to the amino acid sequence of SEQ ID NO: 25;
   u. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26;
   v. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 27;
   w. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 28;
   x. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 29;
   y. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 30;
   z. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 31;
   aa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 32;
   bb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33;
   cc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 34;
   dd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 36;
   ee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 38;
   ff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 39;
   gg. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40;
   hh. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
   ii. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 42;
   jj. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43;
   kk. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44;
   ll. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 45;
   mm. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 46;
   nn. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 47; and
   oo. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 48;
   pp. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:199;
   qq. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:200;
   rr. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO 201;
   ss. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:202;
   tt. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:203;
   uu. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:204;
   vv. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 205;
   ww. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:206;
   xx. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:207;
   yy. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:208;
   zz. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:209;
   aaa. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:232;
   bbb. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:233;
   ccc. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:234;
   ddd. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:235;
   eee. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:236; and
   fff. an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:237.
15. The animal feed additive according to any of embodiments 7 to 10 wherein the polypeptide having superoxide dismutase activity is selected from the group consisting
   a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
   b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
   c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
   d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
   e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
   f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
   g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
   h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
   i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
   j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
   k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
   l. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
   m. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
   n. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
   o. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
   p. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
   q. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
   r. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
   s. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
   t. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
   u. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
   v. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
   w. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
   x. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
   y. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
   z. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
   aa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
   bb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
   cc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
   dd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
   ee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
   ff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
   gg. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
   hh. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
   ii. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
   jj. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
   kk. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
   ll. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
   mm. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
   nn. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
   oo. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
   pp. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
   qq. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
   rr. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
   ss. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
   tt. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
   uu. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
   vv. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
   ww. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
   xx. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
   yy. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
   zz. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
   aaa. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
   bbb. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
   ccc. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
   ddd. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
   eee. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
   fff. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.
16. The animal feed additive according to any of embodiments 7 to 10 wherein the polypeptide having superoxide dismutase activity is selected from the group consisting of
   a. a polypeptide from Trichoderma reesei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 83;
   b. a polypeptide from Aspergillus versicolor having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 84;
   c. a polypeptide from Aspergillus deflectus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 85;
   d. a polypeptide from Aspergillus egyptiacus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 86;
   e. a polypeptide from Westerdykella sp. AS85-2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 49;
   f. a polypeptide from Aspergillus sp. XZ2669 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 50;
   g. a polypeptide from Preussia terricola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 51;
   h. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 52;
   i. a polypeptide from Kionochaeta sp. having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 53;
   j. a polypeptide from Metapochonia bulbillosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 54;
   k. a polypeptide from Xylomelasma sp. XZ0718 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 55;
   l. a polypeptide from Preussia flanaganii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 56;
   m. a polypeptide from Cladobotryum sp having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 18;
   n. a polypeptide from Westerdykella sp-46156 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 57;
   o. a polypeptide from Trichoderma hamatum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 58;
   p. a polypeptide from Mycothermus thermophilus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 59,
   q. a polypeptide from Cephalotrichiella penicillata having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 60,
   r. a polypeptide from Chaetomium megalocarpum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 61;
   s. a polypeptide from Chaetomium thermophilum var. thermophilum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 62;
   t. a polypeptide from Humicola hyalothermophila having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 63;
   u. a polypeptide from Subramaniula anamorphosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 64;
   v. a polypeptide from Sphingobacterium sp. T2 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 65;
   w. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 66;
   x. a polypeptide from Trichoderma lixii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 67;
   y. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 68;
   z. a polypeptide from Aspergillus niveus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 69;
   aa. a polypeptide from Aspergillus templicola having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 70;
   bb. a polypeptide from Pochonia chlamydosporia var. spinulospora having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 71;
   cc. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 35;
   dd. a polypeptide from Trichoderma rossicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 37;
   ee. a polypeptide from Trichoderma sp-54723 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 72;
   ff. a polypeptide from Trichoderma sp-44174 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 73;
   gg. a polypeptide from Metapochonia suchlasporia having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 74;
   hh. a polypeptide from Metarhizium marquandii having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 75;
   ii. a polypeptide from Diaporthe nobilis having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 76;
   jj. a polypeptide from Tolypocladium sp. XZ2627 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 77;
   kk. a polypeptide from Aspergillus japonicus having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 78;
   ll. a polypeptide from Metarhizium sp. XZ2431 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 79;
   mm. a polypeptide from Armillaria ostoyae having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 80;
   nn. a polypeptide from Trichoderma spirale having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 81;
   oo. a polypeptide from Aspergillus elegans having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 82;
   pp. a polypeptide from Trichoderma sinuosum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 210;
   qq. a polypeptide from Trichoderma virens having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 211;
   rr. a polypeptide from Trichoderma harzianum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 212;
   ss. a polypeptide from Fusicolla acetilerea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 213;
   tt. a polypeptide from Plectosphaerella sp. 1-29 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 214;
   uu. a polypeptide from Mariannaea punicea having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 215;
   vv. a polypeptide from Penicillium oxalicum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 216;
   ww. a polypeptide from Colletotrichum sp-71086 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 217;
   xx. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 218;
   yy. a polypeptide from Trichoderma parapiluliferum having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 219;
   zz. a polypeptide from Aspergillus sp. nov. XZ3202 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 220;
   aaa. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 238;
   bbb. a polypeptide from Rhizomucor miehei having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 239;
   ccc. a polypeptide from Mucor sp. XZ2651 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 240;
   ddd. a polypeptide from Amphisphaeriaceae -sp 43674 having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 241;
   eee. a polypeptide from Humicola fuscoatra having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 242; and
   fff. a polypeptide from Valsaria rubricosa having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 243.
17. The animal feed additive according to any of embodiments 7 to 10 comprising one or more polypeptides having superoxide dismutase (SOD) activity wherein the polypeptide having superoxide dismutase activity is selected from the group consisting of:
   a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 5;
   b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 87;
   c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 88;
   d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 89;
   e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 90;
   f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 91;
   g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 92;
   h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 93;
   i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 94;
   j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 95;
   k. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 96;
   l. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 97;
   m. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 98;
   n. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 99;
   o. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 100;
   p. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 101;
   q. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 102;
   r. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 127;
   s. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 128;
   t. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 129;
   u. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 130;
   v. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 131;
   w. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 132;
   x. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 133;
   y. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 134;
   z. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 135;
   aa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 136;
   bb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 137;
   cc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 138;
   dd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 139;
   ee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 140;
   ff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 141;
   gg. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 142;
   hh. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 143;
   ii. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 144;
   jj. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 145;
   kk. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 146;
   ll. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 147;
   mm. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 148;
   nn. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 149;
   oo. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 150;
   pp. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 221;
   qq. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 222;
   rr. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 223;
   ss. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 224;
   tt. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 225;
   uu. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 226;
   vv. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 227;
   ww. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of 228;
   xx. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 229;
   yy. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 230;
   zz. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 231;
   aaa. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 244;
   bbb. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO SEQ ID NO 245;
   ccc. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 246;
   ddd. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 247;
   eee. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 248; and
   fff. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 249.
18. The animal feed additive according to any of embodiments 7 to 17 wherein the feed additive comprises one or more polypeptides having catalase activity and/or further comprising one or more vitamins.
19. The animal feed additive of embodiment 18, wherein the one or more vitamins is a fat-soluble vitamin, preferably vitamin E.
20. The animal feed additive according to any of claims 7 to 19, wherein the polypeptide having catalase activity is selected from the group consisting of:
   a. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6; and
   b. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
   c. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 103;
   d. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 104;
   e. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 105;
   f. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 106;
   g. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 107;
   h. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 108;
   i. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 109;
   j. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 110;
   k. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 111;
   l. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 112;
   m. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 113;
   n. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 114;
   o. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 115;
   p. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 116;
   q. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 117;
   r. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 118;
   s. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 119;
   t. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 120;
   u. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121;
   v. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122;
   w. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 123;
   x. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 124;
   y. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 125;
   z. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 126; and
   aa. a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 127.
21. A method of improving one or more performance parameters in an animal comprising administering to the animal an animal feed or animal feed additive comprising one or more polypeptides having superoxide dismutase (SOD) activity of fungal origin, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).
22. A method of improving or enhancing immune response and/or reducing inflammation and/or for the modulation of the gut flora in an animal comprising administering to the animal an animal feed or animal feed additive comprising one or more polypeptides having superoxide dismutase activity of fungal origin.
23. A method of reducing or eliminating the use of antibiotics administered to an animal feed, comprising administering to the animal an animal feed or animal feed additive comprising of one or more polypeptides having superoxide dismutase activity of fungal origin.
24. The method according to any of embodiments 21 to 23, wherein the animal has experienced heat stress, cold stress, nutritional stress and/or oxidative stress.
25. The method of embodiments 21 to 24 to reduce cellular markers of reactive oxygen species or free radicals.
26. The method of any of embodiments 21 to 25, wherein the polypeptide having superoxide dismutase activity is an EC 1.15.1.1 super oxide dismutase (SOD) of fungal origin.
27. The method of any of embodiments 21 to 26, wherein the polypeptide having superoxide dismutase activity is as defined in any of embodiments 1 to 6.
28. The method of any of embodiments 21 to 27, wherein the polypeptide having superoxide dismutase (SOD) activity is selected from the group consisting of a Cu-SOD, a Zn-SOD, a Mn-SOD, and an Fe-SOD.
29. The method according to any of embodiments 21 to 28, wherein the animal feed or animal feed additive further comprises one or more polypeptides having catalase activity.
30. The method according to any of embodiments 21 to 28, wherein the polypeptide having catalase activity is classified as an EC 1.11.1.6 catalase or as an EP 1.11.1.21 catalase peroxidase.
31. The method according to any of embodiments 21 to 30, wherein the polypeptide having catalase activity is selected from the group consisting of:
   a. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6; and
   b. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7
   c. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 103
   d. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 104;
   e. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 105;
   f. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 106;
   g. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 107;
   h. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 108;
   i. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 109
   j. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 110
   k. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 111
   l. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 112
   m. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 113
   n. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 114
   o. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 115
   p. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 116
   q. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 117
   r. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 118
   s. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 119
   t. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 120
   u. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 121
   v. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122
   w. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 123
   x. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 124
   y. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 125
   z. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 126;
   aa. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 250;
   bb. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 151;
   cc. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 152;
   dd. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 153;
   ee. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 154;
   ff. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 155;
   gg. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 156;
   hh. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 157;
   ii. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 158;
   jj. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 159;
   kk. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 160;
   ll. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 161;
   mm. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 162;
   nn. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 163;
   oo. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 164;
   pp. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 165;
   qq. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 166;
   rr. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 167;
   ss. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 168;
   tt. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 169;
   uu. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 170;
   vv. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 171;
   ww. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 172;
   xx. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 173;
   yy. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 174; and
   zz. a polypeptide with catalase activity having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO 251.
32. Use of an animal feed additive as defined in any of embodiments 7 to 20 as an antioxidant, preferably in feed and feed premixes.
33. Use of an animal feed additive as defined in any of embodiments 7 to 20 for replacing or partially replacing antibiotics in animal feed.
34. An animal feed comprising one or more protein sources and one or more energy sources characterised in that the animal feed further comprises one or more polypeptides having superoxide dismutase (SOD) activity, wherein the animal feed
   a. improves or enhances immune response; or
   b. reduces inflammation;
   when fed to the animal as compared to animals fed a feed composition without the polypeptides; and
      said feed optionally further comprising one or more polypeptides having catalase activity.

### EXAMPLES

### Example 1: Heat Stress Model in Broilers: SOD and CAT Reduce Oxidative Stress Response

### Background

Different stress factors inevitably influence the animal's physiology and performance with varying degrees during the production period. High ambient temperature is one of the most common stressor in modern poultry production resulting in reduced feed intake, body weight gain, and increased mortality. Because of their physiological state and greater metabolic activity, broilers are more susceptible to temperature associated environmental challenges. In addition to its effect on bird performance, the adverse effects of heat stress can range from minimal discomfort to multi-organ damage and failure, including alteration of gut permeability and function due to weakened tight junctions of the epithelial layer. Therefore, heat stress-induced disturbance of gut integrity may lead to leaky gut syndrome thus increasing the susceptibility of birds to enteric pathogens that may lead to systemic bacterial infections. Moreover, heat stress can cause oxidative damage by increasing the formation of reactive oxygen species.

### Hypothesis and Objective

The central hypothesis is that the adverse effects of heat stress on performance and health of chickens can be mitigated by incorporating effective feed additives into poultry diets. Certain compounds that can alter the dysregulating effects of heat stress may help in maintaining gut integrity and function. Therefore, the main objective is to alleviate the impact of heat stress challenge on performance and overall health of broilers by timely supplementation of non-drug dietary additives.

### Experimental Design

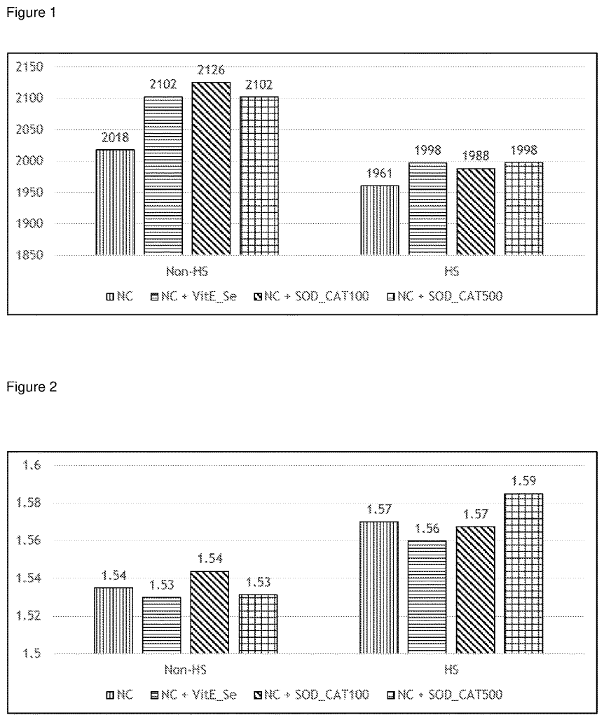

A 35-day trial with only male broiler chicks will be run in floor pens with eight replicate pens per treatment and 20 birds/pen. Birds are randomly assigned to treatment groups in a 2×4 factorial arrangement that includes heat stress (optimal or high heat) and dietary treatments (basal diet or feed additives) as per FIG. 1. A total of 4 animal rooms are used (2 controls and 2 heat stress) each housing half the replicate pens (n=4) per group.

Three birds per pen for each of the three sampling times (3×3=9) are randomly tagged at placement with necktags of different colour (or number) per sampling time to ensure random sampling. At sampling, the two of the three birds with the closest to average BW are selected for sampling and their BW is recorded. If less than two of those birds are available or healthy, other random birds can be selected in the pen to complete the two sampled birds.

Treatment groups including controls and challenge from d 28 to 35 (heat stress) are as follows:

TABLE 2
Treatment groups

| | | Heat Stress |
|---|---|---|
| | | Challenge |
| Treatment | Description | d 28-35 |
| 1. NC | Negative Control (NC) = basal | −−− |
| | diets (corn/soy) | |
| 2. VitE + Se | NC + Vit E + Se (d 0-35) | −−− |
| 3. Mix Low | NC + SOD SEQ: ID 1 500 U/kg + | −−− |
| | CAT SEQ ID: 7 .100 U/kg (d 0-35) | |
| 4. Mix High | NC + SOD SEQ: ID 1 500 U/kg + | −−− |
| | CAT SEQ ID: 7, 500 U/kg (d 0-35) | |
| 5. PC | Positive Control: NC + heat | +++ |
| | stress d 28-35 | |
| 6. VitE + Se | PC + Vit E + Se (d 0-35) | +++ |
| 7. Mix Low | PC + SOD SEQ ID 1, 500 U/kg + | +++ |
| | CAT SEQ ID: 7, 100 U/kg (d 0-35) | |
| 8. Mix High | PC + SOD SEQ: ID 1, 500 U/kg + | +++ |
| | CAT SEQ ID: 7, 500 U/kg (d 0-35) | |

*PC: Vit E - 250 mg/kg; SePlex - 1 mg/kg
**NC: Vit E - 10 mg/kg; SePlex - 0.14 mg/kg

TABLE 3
Treatment identification and doses

| Treatment | Application |
|---|---|
| VitE + Se | Dry, applied in mixer |
| | Vitamin E: 250 mg/kg (ROVIMIX ® E50) |
| | Selenium: 1 mg Se/kg (Sel-Plex ®) |
| Mix Low | Liquid, gently mix the products with distilled water to |
| | complete a total of 1,000 ml/MT (100 ml/100 kg feed) |
| | immediately before application, and spray uniformly into |
| | the mixer, directly into the feed with a micro-fluid nozzle |
| | applicator |
| | SOD: 80 ml/MT (8 ml/100 kg) |
| | CAT: 4 ml/MT (0.4 ml/100 kg) |
| Mix High | Liquid, gently mix the products with distilled water to |
| | complete a total of 1,000 ml/MT (100 ml/100 kg feed) |
| | immediately before application, and spray uniformly into |
| | the mixer, directly into the feed with a micro-fluid nozzle |
| | applicator |
| | SOD: 80 ml/MT (8 ml/100 kg) |
| | CAT: 20 ml/MT (2 ml/100 kg) |

### Handling of Products

Store refrigerated at 4 C.
Gently mix before applying.
Use industrial protective equipment for handling: gloves, mask, googles, and coat.
Mix with distilled water at room temperature to complete 1,000 ml/MT immediately before application, and spray uniformly in the mixer, directly into the feed with a micro-fluid nozzle applicator.

### Heat Stress Protocol

Heat stress protocol is performed as planned during the finisher feeding phase (d 28 to d 35). For the heat stress groups, temperature is maintained at 35±1° C. (10° C. above the recommended temp.) and applied once daily from 10 am to 2 μm, then reduced again to 25 C+/−1 for the remainder of the day. Relative humidity is monitored and maintained at RH<50%. Birds in the separate ‘optimal heat’ group are kept under normal constant recommended temperatures (25° C.+/−1). The birds' (2 birds/pen) rectal temperature was monitored daily with a rectal probe during the heat stress period (d 28-35).

### Feed

The diets consist of corn/soybean in mash as Starter (d 1-21) and Grower (d 22-35) and will be formulated according to commercial specifications for the broilers used (Ross or Cobb) to meet or exceed NRC recommendations (Table 4). Test products are sprayed onto a small amount (20 kg) of the basal diet and mixed with the rest of the batch for each diet.

TABLE 4
Basal diet formulation for Starter and Grower phases.

Basal Diet

| | 0-21 d | 22-35 d |
|---|---|---|
| Ingredient, g/kg | | |
| Corn | 558.80 | 618.20 |
| Soybean meal, CP 48% | 360.00 | 297.00 |
| Vegetable oil | 40.00 | 52.00 |
| Limestone | 5.00 | 4.00 |
| Dicalcium phosphate | 24.00 | 19.00 |
| DL-Methionine (98%) | 3.20 | 2.00 |
| L-Lysine HCl (78%) | 2.80 | 2.00 |
| L-Threonine | 1.30 | 0.80 |
| Salt | 2.50 | 2.50 |
| Vitamin premix¹ | 1.00 | 1.00 |
| Mineral premix² | 1.00 | 1.00 |
| Cholin chloride | 0.50 | 0.50 |
| Total | 1000 | 1000 |
| Chemical composition (Calculated) | | |
| Dry Matter, % | 87.95 | 87.90 |
| Crude Protein, % | 22.25 | 19.50 |
| AMEₙ, kcal/kg | 3060 | 3205 |
| Lysine, % | 1.39 | 1.15 |
| Dig. Lysine, % | 1.23 | 1.02 |
| Methionine + cysteine, % | 1.00 | 0.82 |
| Dig. Methionine + cysteine, % | 0.90 | 0.71 |
| Threonine, % | 0.94 | 0.80 |
| Dig. Threonine, % | 0.81 | 0.68 |
| Calcium, % | 0.96 | 0.78 |
| Available phosphorus, % | 0.47 | 0.39 |

^{1,2}Appended separately. Premix delivers 0.14 mg Se/kg, and 10 mg Vit E/kg

### Parameters Evaluated

1. Performance: Standard performance parameters including body weight (BW), weight gains (BWG), feed intake (FI), and feed conversion ratios (FCR) are evaluated on days 10, 21, 27, and 35 on a per pen basis. Any mortality is recorded daily and feed consumption (+FCR) corrected for accordingly. Average bird performance is calculated based on the number of birds per each pen (i.e. replicate) after adjusting for any mortality.
2. Rectal temperature: The birds' rectal temperature is monitored daily during the heat stress period (d 28-35). Two birds from each replicate pen are selected and tagged for body temperature measurement using a rectal probe daily at a pre-set time range (e.g. 1-2 μm).
3. Antioxidant parameters: Based on the stress challenge employed, blood serum and liver are sampled at 27, 28 and 35 d from two birds of average BW per pen to determine parameters related to oxidative stress:
   a. MDA, CAT, GSH-Px, and SOD: On d 27, two birds per replicate pen are tagged for blood sampling. Blood samples are taken by wing venipuncture on d 27, d 28, and d 35 (alternating sides), and collected into red-topped serum tubes. The tubes are transferred to the main lab and centrifuged at 2,000×g for 5 min to collect sera, which are stored at 20° C. until analysis. Antioxidant analyses in blood samples are carried out with commercial kits according to the manufacturer recommendations.
   b. Optional analyses. Depending on the results of the other parameters, it might be decided to measure Vitamin E, vitamin A, cysteine, cystine, methionine, and glutamine in the liver, using standard HPLC protocols are used to measure vitamins A and E, as well as profile amino acids (separated on a single column). They are not included in the initial budget.
4. Liver bacterial load: At the end of the study (d 35), liver samples are collected from one bird per pen in sterile sampling bags to determine total bacterial load in liver. To establish standard curves for bacterial quantification, pure bacterial (E. coli, and Clostridium perfringens) suspensions are individually cultured in corresponding selective media, the DNA extracted (commercial extraction kits), and its concentration determined (Nano-Drop). The DNA stocks are serially diluted (6 times) and real time quantitative PCR performed to determine the threshold cycle (Ct) value related to each pure sample. The standard curves are established with the DNA concentrations and corresponding relative Ct values. DNA from liver tissue samples are extracted via special kits. The abundance of DNA copies in DNA samples from liver tissue is determined by qPCR. For this purpose, specific primers for bacterial 16S rRNA gene are used, and the bacterial loads per gram tissue are calculated using the generated standard curves.
5. Carcass/body composition (no additional cost): Dual Energy X-Ray Absorptiometry (DXA) analysis is performed on 3 birds per pen on d 35 to assess the effects of products on various measurements of carcass/body composition. These include body fat percentage, fat tissue, lean tissue, and bone mineral density (BMD), in addition to bone mineral content (BMC) and carcass area, both used for calculation purposes.
6. Gene expression: Liver and intestinal (jejunum) tissue samples are taken (2/pen) on d 27, d 28 and d 35 from birds with average pen weight (sampled above for histology). Liver weight is measured and relative weight calculated per bird. Tissues are collected, minced, snap-frozen, and stored at 80° C. until qPCR analysis. Target genes to be measured include immunity-related to heat stress (oxidative stress, heat shock proteins, inflammatory markers), tight junction genes, host defense peptides, and nutrient transporters. The final list of targets is determined based on study outcome. Transcript analysis for a subset of targets is measured in each collected tissue.

### Heat Stress Protocol

Heat stress protocol was performed as planned during the last week of the experiment (d 28 to d 35). For the heat stress groups, temperature was raised and maintained at 35±1° C. (10° C. above the recommended temp.) and applied once daily from 10 am to 2 pm, then reduced again to 25° C.+/−1 for the remainder of the day. Relative humidity was monitored and maintained at RH<50%. Birds in the separate ‘optimal heat’ group were kept under normal constant recommended temperatures (25° C.+/−1). The birds' (2 birds/pen) rectal temperature was monitored daily with a rectal probe during the heat stress period (d 28-35).

### Sampling Procedures

Blood, liver and intestinal (jejunum) tissue samples were taken on d 27 (1/pen), d 28 (2/pen) and d 35 (2/pen) from birds with average pen weight for qPCR and antioxidant assay analyses. The livers were weighed and observed for any pathological findings.

### DXA Analysis

Dual Energy X-Ray Absorptiometry (DXA) analysis was performed on 2 birds per pen on d 35 to assess various measurements of carcass/body composition. Birds were individually wing-banded, sacrificed by cervical dislocation and subsequently defeathered and carcasses stored at −20° C. until DXA analysis. Birds were thawed and 10 birds were scanned at a time using GE Healthcare Lunar Prodigy Advance, System ID PA+130,744 (GE, Madison, Wis.).

### Total RNA Extraction and Reverse Transcription

Total RNA was extracted from individual liver and intestinal tissues using the Direct-zol RNA Kits (Zymo Research) according to the manufacturer's recommendations. Tissue samples were removed from −80° C. and placed on ice. A 20-30 mg aliquot of each sample was weighed, placed into a 2-mL microcentrifuge tube, and kept on ice until homogenization. Total RNA concentration was determined at optical density (OD) of 260 (NanoDrop-1000, Thermo Fisher Scientific, Waltham, Mass.), and RNA purity was verified by evaluating the 260/280 OD ratios. Total RNA was diluted to 0.2 μg/μL in nuclease-free water. Reverse transcription was accomplished using the high capacity cDNA Reverse Transcription kit (Applied Biosystems, Carlsbad, Calif.) following the manufacturer's protocol and the cDNA was stored at 20° C.

### Quantitative Real-Time PCR

Quantitative real-time PCR (qRT-PCR) was performed using an ABI 7500 Fast Real-Time PCR System (Applied Biosystems). The cDNA was diluted 1:20 in nuclease-free water, and 1 μL of the diluted cDNA was added to each well of a 96-well plate. Next, 9 μL of RT-PCR master mix containing 5 μL of Fast SYBR Green Master Mix (Applied Biosystems), 0.5 μL each of 2 μM forward and reverse primers, and 3 μL of sterile nuclease-free water per reaction were added to each well for a final volume of 10 μL. During the PCR reaction, samples were subjected to an initial denaturation phase of 95° C. for 20 s followed by 40 cycles of denaturation at 95° C. for 3 s and annealing and extension at 60° C. for 30 s. Gene expression was analyzed using glyceraldehyde-3-phosphate dehydrogenase (GAPDH) as an endogenous control. Each reaction was run in duplicate. Results from qRT-PCR were analyzed using the 7500 Real-Time PCR software (Applied Biosystems). The primer pairs used are shown in Table 4. Average gene expression relative to the GAPDH endogenous control for each sample was calculated using the 2^{-ΔΔct} method (Livak and Schmittgen, 2001).

### Antioxidant Analysis

Based on the stress challenge employed, blood serum and liver samples were analyzed to determine thiobarbituric acid reactive substances (TBARS), catalase (CAT), glutathione peroxidase (GPx), and superoxide dismutase (SOD) activities on d 27, d 28 and d 35. Antioxidant analyses were carried out with commercial assay kits (Cayman, Ann Arbor, Mich.).

### Statistical Analysis

Data were subjected to a 2-way ANOVA using the GLM procedure of JMP (Pro13). The models included heat stress (optimal and high heat) and dietary treatments (basal diet or feed additives) as the main factors, and the 2-way interactions. Post hoc testing was only carried out for significant interactions and was performed using simple effect analysis. The probability P<0.05 was considered significant unless otherwise noted.

TABLE 5
Primer sequences for qPCR analysis.

| Target Gene | Forward | Reverse |
|---|---|---|
| Intestinal tissue: | | |
| Occludin | TCATCGCCTCCATCGTCTAC | TCTTACTGCGCGTCTTCTGG |
| ZO-1 | TGTAGCCACAGCAAGAGGTG | CTGGAATGGCTCCTTGTGGT |
| Muc-2 | CAGCACCAACTTCTCAGTTC | TCTGCAGCCACACATTCTTT |
| TFF-2 | TGGTCCCCCAGGAATCTCA | CACCGACGCATTGAAGCA |
| PepT1 | CCCCTGAGGAGGATCACTGTT | CAAAAGAGCAGCAGCAACGA |
| SI | CGCAAAAGCACAGGGACAGT | TCGATACGTGGTGTGCTCAGTT |
| IAP | GAGCCTACACCAGCATCCTC | GCTGCCTGTAGTCCTTGTCC |
| NaPi-Ilb | GAAAGTGGTGAAGATGCC | AAGTATGAGACCGATGGC |
| SG LT-1 | GGGACAGTAGGTGGATTCTTT | CACCAATCGGCCACCAA |
| | CTG | |
| Liver: | | |
| CAT | TGAAGCGGTGGCAAAGC | GCCAGCACCAGTGGTCAAG |
| GSH-Px | TTGTAAACATCAGGGGCAAA | TGGGCCAAGATCTTTCTGTAA |
| SOD | AGGGGGTCATCCACTTCC | CCCATTTGTGTTGTCTCCAA |
| GST | GCCTGACTTCAGTCCTTGGT | CCACCGAATTGACTCCATCT |
| Intestine & liver: | | |
| IL-1β | CACAGAGATGGCGTTCGTTC | GCAGATTGTGAGCATTGGGC |
| IL-4 | GCTCTCAGTGCCGCTGATG | GAAACCTCTCCCTGGATGTCAT |
| IL-6 | TTCGACGAGGCAAGGAACC | AGGTCTGAAAGGCGAACAGG |
| IL-10 | GCTCTCCTTCCACCGAAACC | GGAGCAAAGCCATCAAGCAG |
| TLR-2 | GCGAGCCCCCACGAA | GGAGTCGTTCTCACTGTAGGAGACA |
| TLR-4 | TCCGTGCCTGGAGGTAAGT | TGCCTTGGTAACAGCCTTGA |
| LITAF | TGTTCTATGACCGCCCAGTTC | AGACGTGTCACGATCATCTGGTTA |
| iNOS | CCTGTACTGAAGGTGGCTATT | AGGCCTGTGAGAGTGTGCAA |
| | GG | |
| IFN-Y | GCATCTCCTCTGAGACTGGC | GCTCTCGGTGTGACCTTTGT |
| HSP70 | CATCAAGCGTAACACCACCATT | CCAGCAAGTTGTTGTCCTTTGTCATA |
| | CC | |
| HSP90AA1 | AGAAAAGGTTGTTGTGTCCAAT | CAGCCTCTGCCTTCTGCCTCA |
| | CGC | |
| Lysozyme | GAAGCGTCACGGACTTGATAA | CCCATCGGTGTTACGGTTTG |
| | CTA | |
| Housekeeping | House Keeping genes | |
| β-actin | CAACACAGTGCTGTCTGGTGG | ATCGTACTCCTGCTTGCTGATCC |
| | TA | |
| GAPDH | CCTAGGATACACAGAGGACCA | GGTGGAGGAATGGCTGTCA |
| | GGTT | |

*Not including host defense peptides.

TABLE 6
Sampling schedule

| Time point | # of birds | Samples | Analyses |
|---|---|---|---|
| 27 d, 28 d | 128/day | Blood | Antioxidant parameters |
| and 35 d; | (2 birds | serum | |
| start at 2pm | per pen) | Liver | Antioxidant parameters, gene |
| | | | expression (oxidative, immune |
| | | | genes); bacterial load at d 35 |
| | | Jejunal | gene expression (oxidative, immune, |
| | | mucosa | nutritional, gut integrity genes) |

### Results

Experimental results are shown in FIGS. 1 to 15 and can be summarized as follows:

### Performance Parameter:

Performance parameter are shown in

### FIG. 1: BWG, d 0-35

### FIG. 2: FCR, d 0-35

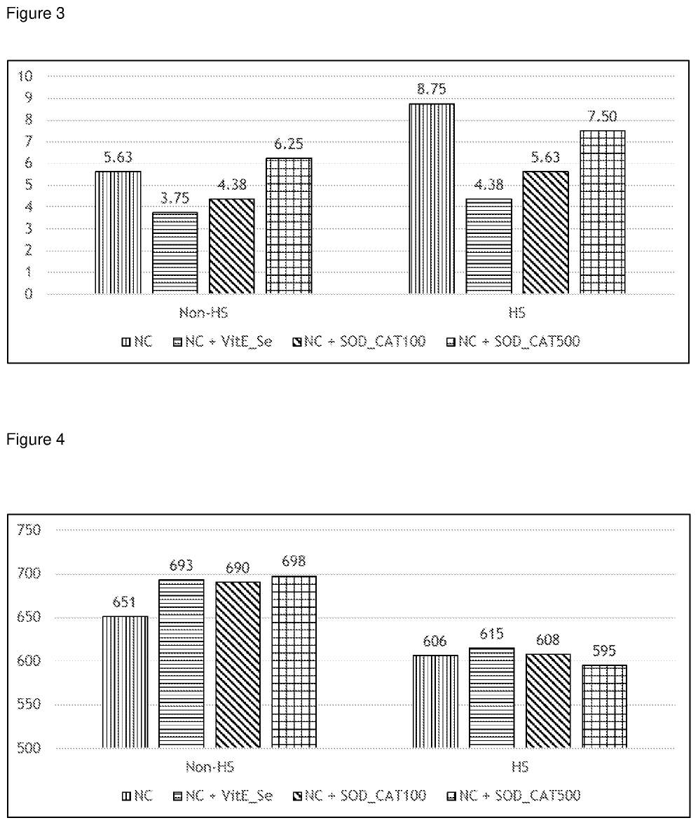

### FIG. 3: Mortality, d 0-35

### FIG. 4: BWG 27-35 d

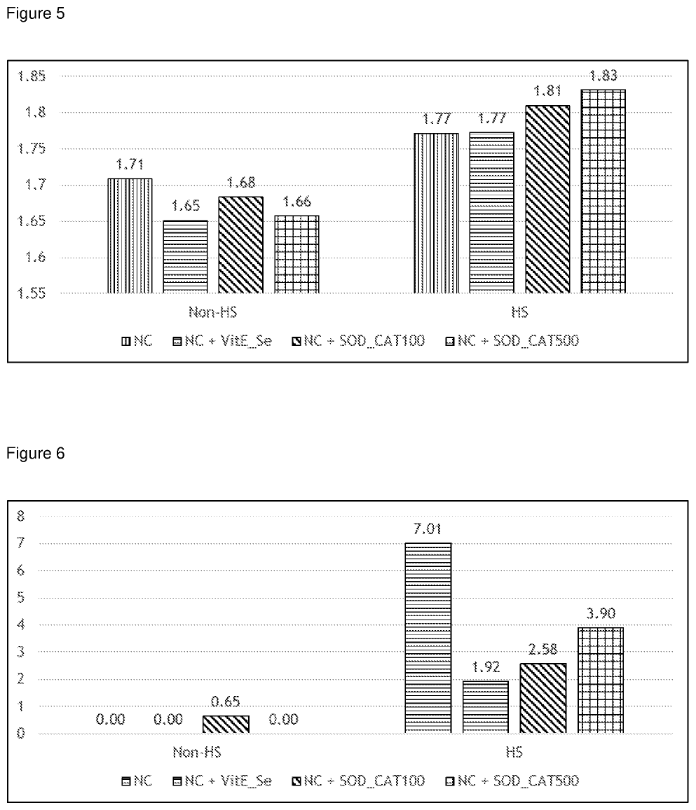

### FIG. 5: FCR 27-35 d

### FIG. 6: Mortality 27-35 d

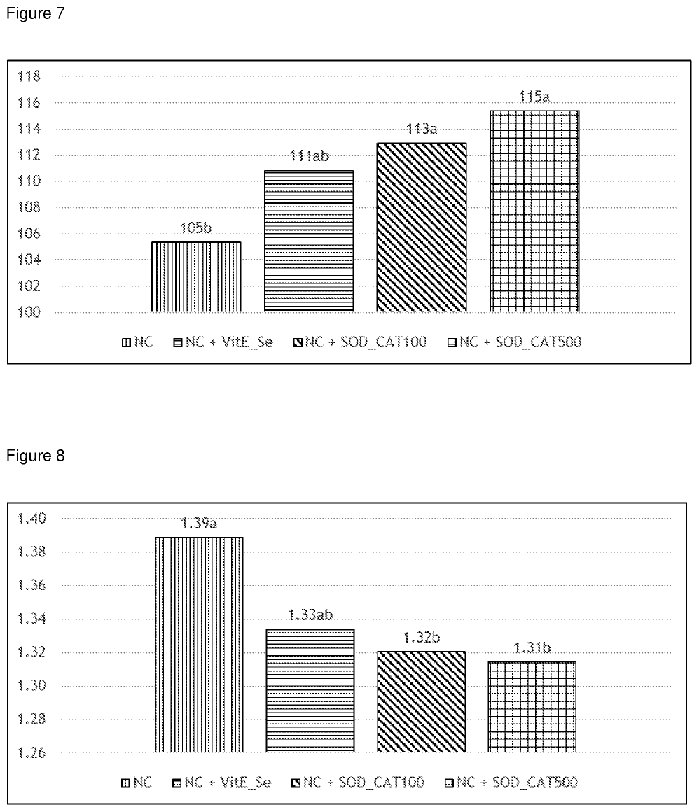

### FIG. 7: BWG 0-7 d

### FIG. 8: FCR 0-7 d

There was an effect of SOD 400+CAT 100 on BW gain and feed intake in the overall period.
A high performance effect of enzymes on BWG and FCR was present in the first week. As reported in FIGS. 7 & 8, body weight gain (BWG) and feed conversion ratio (FCR) were improved in birds fed the diets containing either Pro. Low (T3&T7) or Pro. High (T4&T8) compared to birds fed the control diet.
Feed intake (FI) and mortality were not affected among the treatment groups during the first week (d 0-7). In particular, a numeric reductions in mortality during heat stress and over the entire trial could be observed.
Birds fed the diets containing Vit E+Se, Pro. Low, and Pro. High had higher Feed intake compared to the control group and also tended to have higher BWG between d 7-14. Similarly, BW and FI were higher in birds fed those diets containing Vit E+Se, Pro. Low, and Pro. High between d 0-14.
Birds fed antioxidant enzymes or Vit E & Se did not perform better during heat stress. However, numerically there was less mortality in birds fed antioxidants (enzymes or Vit E& Se) compared to the control during HS.

The data presented above show that an animal feed additive comprising at least one or more polypeptides according to the present invention improves one or more performance parameters selected from the group consisting of
Feed Conversion Ratio (FCR),
Growth Rate (GR), Body Weight Gain (WG),
Mortality Rate (MR).

### Other Parameters:

With respect to markers for oxidative and cellular stress and immune function a significant interaction was observed in the HSP90 gene expression between dietary and heat stress treatments. In particular, the heat challenge significantly upregulated the expression of GPx, SOD, HSP70, HSP90, and TLR4, and downregulated that of GST, IL-10, TNFα, and IFNγ in the liver on d 27/28.

### A) Liver Gene Expression

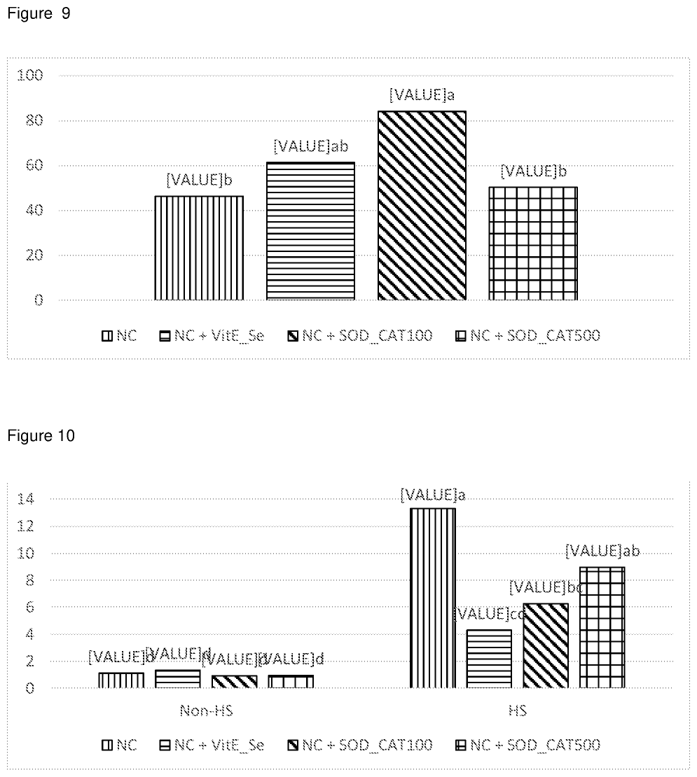

The dietary treatments had significant increase in liver GPx (pre-heat stress) with the lowest doses of enzymes (FIG. 9—liver GPx, nmol/min/mL (measured day 27))

B) Measurement of Heat Shock Proteins (HSP), which are Indicators of Cellular Stress.

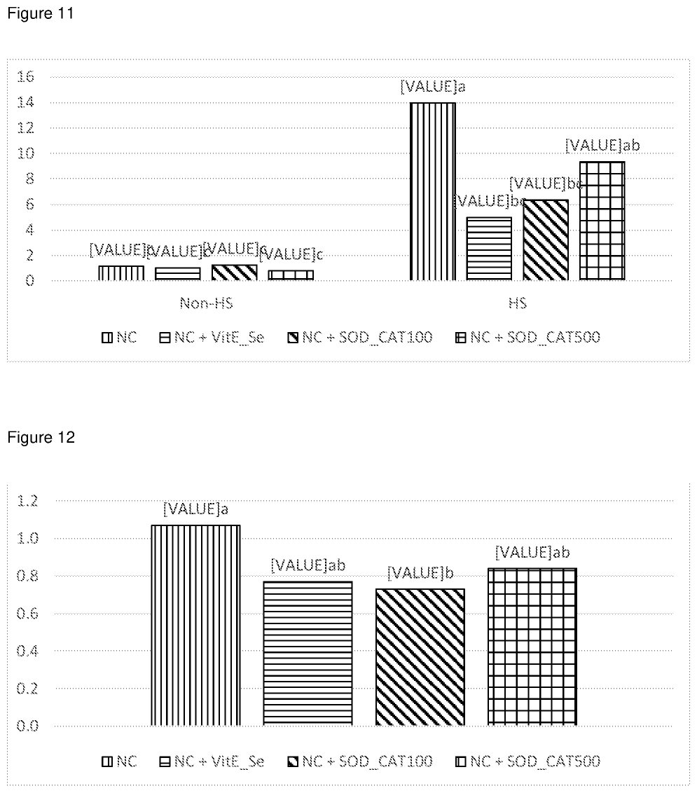

With respect to HSP
a significant reduction of jejunum gene expression of HSP70 (FIG. 10—(day 28)) and liver gene expression of HSP70 (FIG. 11—(day 35)) during heat stress at the lowest dose of enzymes and
a numeric reduction in liver gene expression of HSP90 during heat stress was observed.
   C) Immune function.

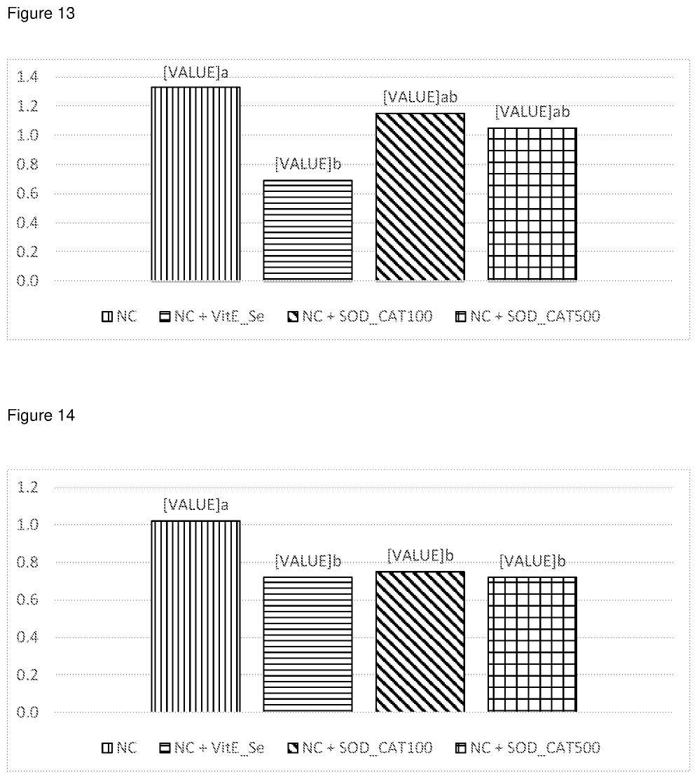

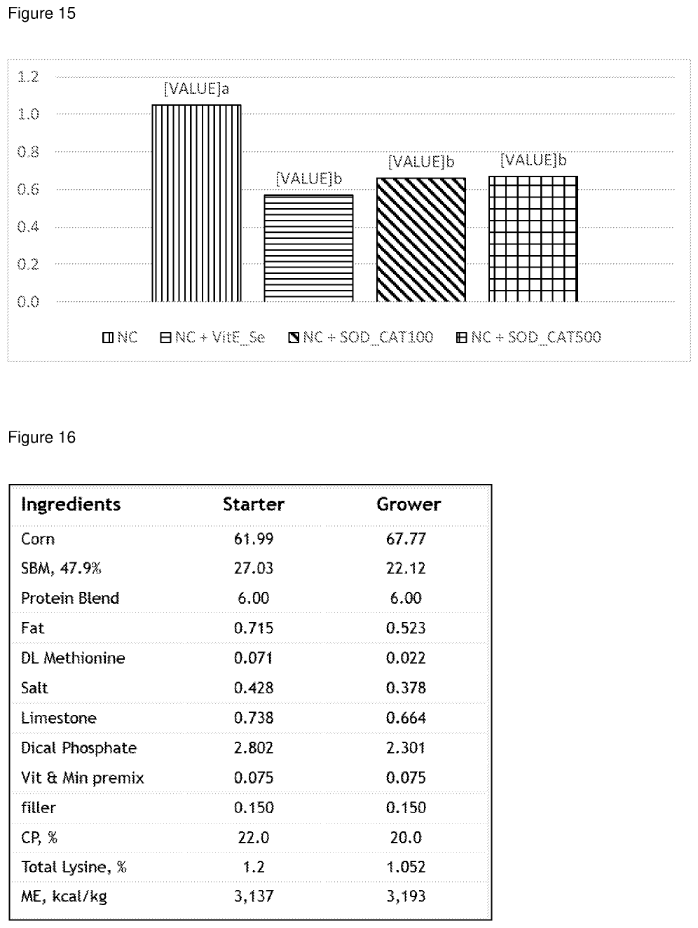

The enzyme according to the present invention also show a positive effect on immune function. The treatments show a significant reduction of liver iNOS prior to heat stress challenge (FIG. 12—liver iNOS (mediator of inflammation) pre-heat stress (d 27)) or TLR2, IL-1B, IL-10, TNFa, and INFy (FIG. 13—liver gene expression of IL-10 (anti-inflammatory) during heat stress (d 35); FIG. 14—liver gene expression of TNF alpha (pro-inflammatory) during heat stress (d 35); FIG. 15 liver gene expression of INF gamma (pro-inflammatory) during heat stress (d 35)).

The results on all these other parameters clearly show that the enzymes according to the present invention help
to reduce
   heat-, cold-, nutritional- and oxidative stress,
   cellular markers of reactive oxygen species or free radicals,
   inflammation and
to improve or enhance immune response in the animal.

### Example 2: Nutritional Model in Piglets

Experimental Unit: Slatted Floor Pens, 4 piglets per pen (2 barrows & 2 gilts per pen)

Feed Type: Starter (Day 0-21) and Grower (Day 21-42). MASH feed is employed, using a corn-soybean type commercial ration formulation with an increase of 5-6% crude protein compared to normal commercial rations. Test material is added to the feed beginning on Day 0 and continuing through Day 42.

TABLE 7
Treatments (Days 0-42)

| | SOD (U/kg) | CAT (U/kg) |
|---|---|---|
| Treatments | SEQ ID NO: 1 | SEQ ID NO: 7 |
| Positive Control (PC)* | 0 | 0 |
| Negative Control (NC, 22% CP) | 0 | 0 |
| NC + Low SOD | 500 | 0 |
| NC + High SOD | 2500 | 0 |
| NC + Low CAT | 0 | 100 |
| NC + High CAT | 0 | 500 |
| NC + Low SOD + Low CAT | 500 | 100 |
| NC + Low SOD + High CAT | 500 | 500 |

*PC: Avilamycin at 90.7 g/lb
12 replicates per treatment with 4 piglets (2 barrows + 2 gilts) per replicate
Weaning age 21 d

Replication/piglets per treatment: Treatments 1-8 fed to 12 replicates each of 4 piglets (2 barrows+2 gilts) per replicate.

TABLE 8
Test measurements

| Data/Sample | | | |
|---|---|---|---|
| Collected | When | Description | Measurements |
| Growth | Day 0, 21, | Individual body weights | FI, BW, BWG |
| performance | and 42 | (0, 21, and 42 days) and | to calculate |
| | | Feed Intake (Days 0-21 | adjusted FCR |
| | | and 21-42) | |

Piglet Source: Piglets (post-weaned, at 21 days of age, called Day 0) are collected in early am (day of placement or Day 0), weighed (2 barrows & 2 gilts per pen), and assigned to each experimental pen. Each piglet is weighed individually and assigned to a pen, with very high and very low body weights excluded from the study. After all piglets are assigned to a pen, the body weights are checked to ensure no treatment is more than 1 standard deviation from another treatment.

The basal diet is shown in FIG. 16.

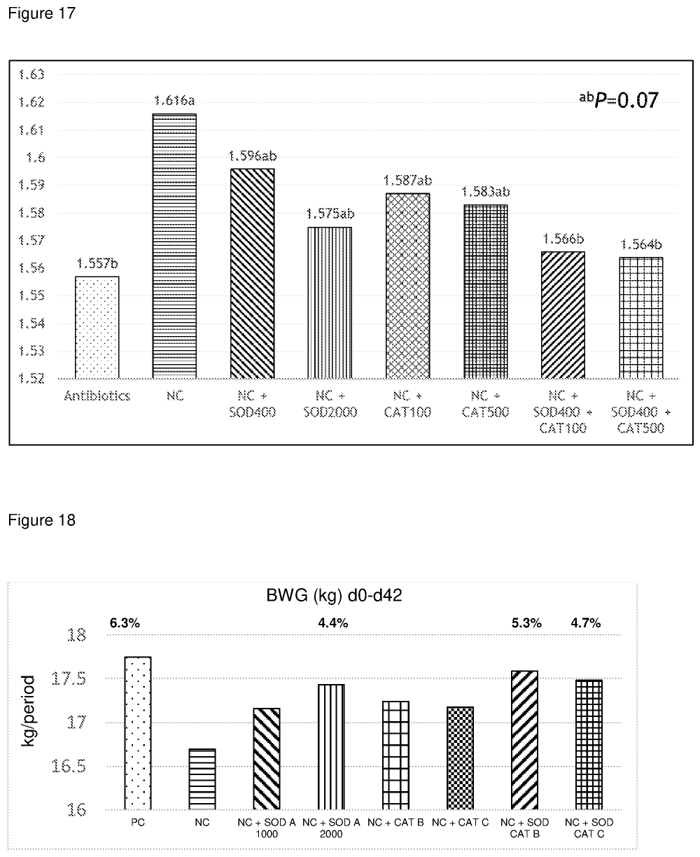

Experimental results on FCR (d 0-42) are shown in FIG. 17:
There was significant difference observed in growth performance between positive and negative control.
Combining SOD & CAT or feeding CAT and SOD alone gave similar performance to the antibiotic treatment and significantly better than NC.

### Example 3: Efficacy of Antioxidant Enzymes (SOD and CAT) Supplemented at Different Doses Alone or in Combination on the Growth Performance of Early Weaned Piglet

### Summary

The effect of antioxidant enzymes (SOD and CAT) alone at different doses and in combination on the growth performance of early weaned piglets was evaluated in a trial run. A total of eight treatments were tested during the study: 6 test groups: SOD (Product A, SEQ ID No. 1 at 1250 and 2500 U/kg), CAT (Product B, SEQ ID NO 7 and Product C, SEQ ID NO 250 at 100 U/kg) and 2 combinations (A 500 U/kg+B 100 U/kg; and A 500 U/kg+B 100 U/kg); were compared against a Positive Control feed (fed with Avilamycin at 90.7 g/lb) and a Negative Control (standard ration). Each of treatments contained 12 replicates per treatment randomly assigned and containing 5 male piglets per replicate for a total number of 480 animals on study. Piglets were randomly assigned to treatments on Trial Day 0 (post-weaning at 21 days of age). At 42 days of age, live performance (growth weight gain and feed conversion) was determined. All rations were formulated with an increase of approximately 5-6% additional crude protein above normal commercial levels. Blood samples were collected at day 0, 14 and 42 to evaluate oxidative stress parameters at DSM. Gut tissue samples at the duodenum and ileum to evaluate gene expression linked to oxidative stress, and intestinal content (ileum and caecum), to study the microbiome population, were collected from 12 animals at the beginning of the trial (baseline) and one animal per pen (12 piglets×8 treatments) at day 14 and at day 42. For the entire grow-out period (Days 0-42), body weight gain showed significant improvement over the Negative Control group when piglets were fed diets containing 2000 U/kg of SOD (Product A), 100 U/kg of CAT B, and both combination of 400 U/kg of SOD (Product A) with 100 U/kg of both CAT products B & C, with the greatest improvements coming with the combination of 400 U/kg of SOD with 100 U/kg of CAT B, which also performed statistically similar to the Positive Control ration that contained Avilamycin at 90.7 g/lb. Feed conversion improved significantly between Positive Control and Negative Control.

### Test System

The commercial-simulated test model employed in this study used piglets reared under a normal swine industry Starter diet (Trial Day 0-21) and Grower diet (Trial Day 21-42), reared on slatted floors. Ration formulations were conducted via computer-generated linear regression program that simulates formulations conducted during practical swine production techniques. Treatments were tested in male piglets. Piglets were continuously fed their experimental diets from time of placement on Trial Day 0 to Trial Day 42.

TABLE 9
Treatments

| Treatment | | |
|---|---|---|
| Number (T) | Treatment Description | Dose inclusion |
| 1 | Neg. control | — |
| 2 | PC: Avylamycin | 90.7 g/lb |
| 3 | NC + Product A | A = 1000 U/kg |
| 4 | NC + Product A | A = 2000 U/kg |
| 5 | NC + Product B | B = 100 U/kg |
| 6 | NC + Product C | C = 100 U/kg |
| 7 | NC + Product | A = 400 U/kg |
| | A + Product B | B = 100 U/kg |
| 8 | NC + Product | A = 400 U/kg |
| | A + Product C | C = 100 U/kg |

Piglets were weighed and randomly placed into each pen on Trial Day 0 and fed their respective diets. Each pen had sufficient floor density, feeder and waterer space for each grow-out area for the piglets. Following 42 days of grow-out, piglets were weighed, feed consumption determined, and feed conversion (feed consumed/body weight) calculated.

Materials and Methods

Experimental Design

A total of 500 male piglets (a sufficient number to ensure availability of at least 480 healthy male piglets for the conduct of the study) were obtained from a swine breeder on Trial Day 0 (upon weaning, when the piglets were 21 days of age). Piglets were fed their respective treatment feed ad libitum from Trial Day 0 to Trial Day 42.

### Housing and Daily Observations

Each experimental test unit of swine pens was housed in separated pens, located in a room containing forced air heaters with a cross-house ventilation system. Piglets were placed in a 6 ft×6 ft pen floor area with a minimum of 9.0 ft² per piglet (without feeder and waterer space) provided. At least two nipple drinkers per pen (via well water) provided water. Piglets were observed daily for overall health, behavior and/or evidence of toxicity, and environmental conditions. Temperature in the test facility was checked daily. Drinking water and feed were confirmed to be provided ad libitum. No type of medication (other than test material) was administered during the entire feeding period.

### Data and Observations

Live performance body weights and feed intakes were collected on Days 0, 14, 21, and 42 during the growing period. Weight gain, feed intake, feed:gain ratio (feed efficiency) were calculated for 0-42 days of age between weaning and Trial Day 42. Differences between piglets fed control and test groups were statistically evaluated at P<0.05 in a typical ANOVA analysis of variance test model, employing Treatment×Replicate RCB (Randomized Complete Block). Control group was considered to be the following: Treatment 2, containing Avilamycin at 90.7 g/lb.

At the end of the study, all piglets were humanely euthanized and were disposed of according to local regulations via on-farm composting techniques.

### Diet and Water Administration

Diets were fed in two feed phases: Starter diet (0-21 days of age) and Grower diet (21-42 days of age). All diets were offered ad libitum, without restriction. Fresh well water (from the research facility deep well) was provided ad libitum.

TABLE 10
Feed Formulation Parameters

| | Starter (PC) | Grower (PC) |
|---|---|---|
| Nutrient | 0-21 days | 21-42 days |
| Metabolizable Energy (kcal/kg) | 3150 | 3200 |
| Metabolizable Energy (kcal/#) | 1425 | 1450 |
| Protein (%) | 22.000 | 20.000 |
| Lysine (%) | 1.200 | 1.052 |
| Methionine + Cystine (%) | 0.800 | 0.700 |
| Total Phosphorus (%) Min | 0.941 | 0.819 |
| Available Phosphorus (%) | 0.650 | 0.550 |
| Total Calcium (%) | 0.750 | 0.650 |
| Dietary Sodium (%) | 0.200 | 0.180 |
| Dietary Choline (g/kg) | 1.305 | 1.211 |

### Results

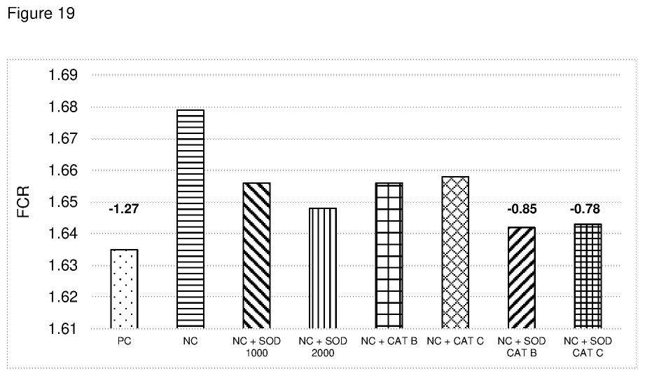

For the entire grow-out period (Days 0-42), body weight gain (FIG. 18) showed significant improvement over the Negative Control group (no added test materials) when piglets were fed diets containing 2000 U/kg of Product A, 100 U/kg of Product B, and a combination of 400 U/kg of Product A with 100 U/kg of both Product B and Product C, with the greatest improvements coming with the combination of 400 U/kg of Product A with 100 U/kg of Product B, which also performed statistically similar to the Positive Control ration that contained Avilamycin at 90.7 g/lb. As shown in FIG. 19, feed conversion improved between Treatment 2 (Positive Control) and Treatment 1 (Negative Control) over the period of d0-d42. In particular, inclusion of SOD+CAT (independently of the candidate B or C) in the ratio 400:100 U/kg showed significant improvement on FCR performance.

In conclusion, the use of a combination of 80 ml/MT of Product A and either 4 ml/MT or 20 ml/MT of Product B appeared to significantly improve 0-42 days of age body weight gains and feed conversion as compared to a standard control ration. Therefore, when commercial piglets are grown on concrete slat floors, this combination appears to have the great potential to result in better live performance.

Analysis and calculation of body weight coefficient of variation at day 14 shows a significant effect of the treatment according to the invention on CV of BW. The data is shown in table 11. The results show that the enzymes according to the invention improve flock uniformity compared to the control T1.

TABLE 11
Body weight coefficient of variation at day 14

| | Treatment | | | | | | | |
|---|---|---|---|---|---|---|---|---|
| Criterion | T1 | T2 | T3 | T4 | T5 | T6 | T7 | T8 |
| Average Body Wt. (kg) Day 14 | 8.998 | 9.215 | 9.133 | 9.149 | 9.111 | 9.105 | 9.182 | 9.173 |
| Stat ¹ | b | a | ab | ab | ab | ab | a | a |
| Feed Conversion Corrected Day 0-14 | 1.232 | 1.215 | 1.223 | 1.220 | 1.223 | 1.224 | 1.218 | 1.217 |
| Stat ¹ | a | a | a | a | a | a | a | a |
| Mortality % Day 0-14 | 1.667 | 0.000 | 0.000 | 0.000 | 0.000 | 0.000 | 1.667 | 0.000 |
| Stat ¹ | a | a | a | a | a | a | a | a |
| Average Body Wt. Gain (kg) Day 0-14 | 3.503 | 3.720 | 3.562 | 3.574 | 3.596 | 3.578 | 3.710 | 3.607 |
| Stat ¹ | b | a | ab | ab | ab | ab | a | ab |
| Body Weight Coefficient of Variation (Day 14) | 6.023 | 4.700 | 4.552 | 5.060 | 5.474 | 4.952 | 5.516 | 5.791 |
| Stat ¹ | c | ab | a | abc | abc | abc | abc | bc |
| Feed Intake (kg/pig) Day 0-14 | 4.36 | 4.52 | 4.36 | 4.36 | 4.40 | 4.38 | 4.57 | 4.39 |
| Stat ¹ | a | a | a | a | a | a | a | a |

¹ Means within a row without a common superscript are significantly different (P < 0.05) as determined by Least Significant Difference.

### Example 4: Effect of Antioxidant Enzymes (SOD and CAT) Alone or in Combination on Plasmatic Antioxidant Enzymes, Oxidative Stress Index and Intestinal Gene Expression Response of Weaned Piglets (Based on Animal Trial of Example 3)

### Summary

The objective of this study was to evaluate anti-oxidant enzymes supplementation (SOD and CAT) alone and in combination in early weaned piglets, and if these enzymes can potentially alleviate the oxidative stress impact on weaned piglets and consequently improve the growth performance. In the in vivo trial (example 3), plasma samples were collected at day 0 (baseline, weaning moment), day 14 and day 42 of the study for additional analysis.

Piglets supplemented with CAT alone showed a significant increase in the plasmatic antioxidant activity (PAT). At day 42, also piglets supplemented with CAT showed a significant increase of GPx activity. Gene expression showed significant modulation at day 14. In duodenum at day 14, the supplementation of antioxidant enzymes (SOD+CAT) showed a downregulation of the genes that encode NQO1 (protecting the cells against Oxidative Stress OS) and the anti-oxidant enzyme GSTP1 (p=0.07 and p=0.005 respectively) suggesting that diet supplementation with a combination of SOD and CAT decreases stress protein expressions at the beginning of small intestine of weaned piglets. SCARA3 showed also a significant increase in the same group (p=0.003), avoiding accumulation of ROS. At the duodenum, the gene that encodes the antioxidant enzyme GPX1 showed a significant upregulation in piglets supplemented with CAT alone compared to control group and those results are in agreement with the GPx plasmatic activity found. And at the ileum level, supplementation of CAT and SOD alone showed a upregulation on the protecting protein NQO1 but a downregulation on HSP70, and piglets supplemented with SOD+CAT showed a downregulation on the genes NOX5, oxidative stress marker, and CAT, antioxidant enzyme. Those data provide evidence that diet supplementation with a SOD, CAT and the combination of both enzymes, potentially decrease oxidative stress status in weaned piglets.

TABLE 12
Test System

| Treatment | | |
|---|---|---|
| Number (T) | Treatment Description | Dose inclusion |
| 1 | Neg. control | — |
| 2 | PC: Avylamycin | 90.7 g/lb |
| 3 | NC + Product A | A = 1000 U/kg |
| 4 | NC + Product A | A = 2000 U/kg |
| 5 | NC + Product B | B = 100 U/kg |
| 6 | NC + Product C | C = 100 U/kg |
| 7 | NC + Product | A = 400 U/kg |
| | A + Product B | B = 100 U/kg |
| 8 | NC + Product | A = 400 U/kg |
| | A + Product C | C = 100 U/kg |

TABLE 13
Biological samples and analysis

| | | | Enzymes | | |
|---|---|---|---|---|---|
| Type of | | | activity: SOD, | Vitamins | Gene |
| sample* | Treat. | Nb sample/treat. | CAT GPX, OSI | A & E | expression |
| Plasma d 0 | 0 | 12 | X | | |
| | 0 | 12 | | X | |
| Plasma d 14 | 1, 4, 6, 8 | 12 × 4 = 48 | X | | |
| | 1, 4, 6, 8 | 12 × 4 = 48 | | X | |
| Plasma d 42 | 1, 4, 6, 8 | 12 × 4 = 48 | X | | |
| | 1, 4, 6, 8 | 12 × 4 = 48 | | X | |
| Duodenum | 1, 4, 6, 8 | 12 samples x4 | | | x |
| and Ileumd 0 | | treatments x 2 | | | |
| | | tissues = 96 | | | |
| Duodenum | 1, 4, 6, 8 | 12 samples x4 | | | x |
| and Ileum | | treatments x 2 | | | |
| d 14 | | tissues = 96 | | | |
| Duodenum | 1, 4, 6, 8 | 12 samples x4 | | | x |
| and Ileum | | treatments x 2 | | | |
| d 42 | | tissues = 96 | | | |

### Methods

### Oxidative Stress PCR Array

Duodenum and ileum were collected for gene expression measurement of genes related to oxidative stress, at day 0 and day 14. The list of the genes evaluated is presented in the table 14 below.

TABLE 14
Genes related to OS

| Gene | | |
|---|---|---|
| Symbol | Official Full Name | Function |
| NQO1 | NAD(P)H dehydrogenase, | Protection against OS |
| | quinone 1 | |
| TXN | Thioredoxin | Anti-oxidant enzymes |
| NOX5 | NADPH oxidase, EF-hand | Oxidative stress marker |
| | calcium binding domain 5 | |
| HSP70 | Heat shock protein 70 | Protection against OS |
| GPX1 | Glutathione peroxidase 1 | Anti-oxidant enzymes |
| SCARA3 | Scavenger receptor class | Protection against OS |
| | A, member 3 | |
| GPX3 | Glutathione peroxidase 3 | Anti-oxidant enzymes |
| GSTP1 | Glutathione S-transferase P | Anti-oxidant enzymes |
| SOD3 | Superoxide dismutase 3, | Anti-oxidant enzymes |
| | extracellular | |
| CAT | Catalase | Anti-oxidant enzymes |
| PRDX5 | Peroxiredoxin 5 | Anti-oxidant enzymes |

Total RNA was extracted from tissues (stored at −20° C. in RNA later) by lysing tissue with FastPrep® 24 (MP Biomedicals, Illkirch, France), using the phenol-chloroform method (TRIzol reagent; Invitrogen, Invitrogen, Cergy Pontoise, France) followed by purification using RNeasy columns by automated method with the Qiacube HT (Qiagen, Courtaboeuf, France). The concentration of RNA was measured by NanoDrop ND-1000 Spectrophotometer (Thermo Fisher Scientific, Illkirch, France) and the purity was estimated by A260/A280 ratio. RNA integrity was assessed by using the Agilent 2100 Bioanalyzer (Agilent Technologies, Basel, Switzerland). The threshold of the RNA Integrity Number (RIN) was set at 7.5 to validate sufficient quality of the RNAs.

The reverse transcription was performed using RT² First Strand Kit (Qiagen, Courtaboeuf, France) with 500 ng of total RNA. The reaction mix was incubated 5 minutes at 42° C. for genomic DNA elimination, followed by the reverse transcription 15 minutes at 42° C. The inactivation of the enzyme was performed by heating 5 minutes at 95° C. The resultant cDNAs were amplified with RT² SYBR Green Mastermixes (Qiagen, Courtaboeuf, France) for real-time PCR. The expression of target genes was normalized with housekeeping genes listed in the table below:

| Gene Symbol | Official Full Name |
|---|---|
| UBC | ubiquitin C |
| GAPDH | glyceraldehyde-3-phosphate dehydrogenase |

The thermal cycling was run on Light Cycler 96 (Roche Diagnostics, Meylan, France) with the following program: 95° C. 10 min, followed by 40 cycles of denaturation at 95° C. 15 seconds and hybridization/elongation at 60° C. 1 min. The Delta Ct method was used to determine expression of target genes (Livak and Schmittgen, 2001)

### Oxidative Stress Index: FRAS5

An oxidative stress index was calculated as the ratio of reactive oxygen metabolites (ROM) and Plasma antioxidant capability (PAT) determined in plasma and liver lysate samples. ROM and

PAT were analyzed using commercially methods (d-ROM fast test and PAT test) on FRAS5 instrument (Innovatics Laboratory, Philadelphia, USA). For d-ROM test, 10 μL of sample was mixed with an acidic buffer. Then, a chromogen was added, and measured photometrically at 505 nm. The results are expressed in UCarr (1 UCarr=0.08 mg of H2O2/dl). For PAT test, 10 μl of sample was added in a colored solution (mix of source ferric ions and chromogen). The intensity of chromatic change was measured at 505 nm. The results are expressed in UCor (1 UCor=1.4 μM of Vitamin C equivalent). The oxidative stress index was calculated as the ratio of d-ROMs test value to PAT test value.

### Total Proteins Determination

The concentration of total proteins was determined in liver lysate and plasma, using the Pierce™ BCA Protein Assay Kit (Thermo Fisher Scientific, Illkirch-Graffenstaden, France). The samples were processed following the supplier's recommendations. Plasma samples were diluted 100 times. 25 μL of standards or samples were added to 200 μL of working reagent and incubated 30 minutes at 37° C. The colorimetric intensity was measured at 562 nm. Results are expressed in μg of total protein per mL of sample.

### Plasma CAT Activity

The activity of the catalase was determined using the Amplex® Red Catalase Assay Kit (Molecular Probes A22180). Briefly 25 μL of standards, plasma (adjusted at 1 μg of protein/well) was mixed with 25 μL of 40 μM H₂O₂ solution and incubated for 30 minutes at room temperature. 50 μL of the Amplex Red/HRP working solution was added to each microplate well containing the samples and standards and incubated 30 min at 37° C. protected from light. The absorbance was measured at 560 nm, and the concentration of CAT in the samples was calculated based on the standard curve.

### Plasma SOD Activity

The activity of the superoxide dismutase was determined using the SOD determination KIT (Sigma 19160) and quantity if reagent in each well is described in table 15 below:

TABLE 15

| | SOD | | | | |
|---|---|---|---|---|---|
| | standard | Samples | Blank1 | Blank2* | Blank 3 |
| SOD Standard | 20 μl | — | — | — | — |
| Sample solution | — | 20 μl | — | 20 μl | — |
| ddH₂O | | | 20 μl | | 20 μl |
| WST working solution | 200 μl | 200 μl | 200 μl | 200 μl | 200 μl |
| Enzyme working | 20 μl | 20 μl | 20 μl | | |
| solution | | | | | |
| Dilution buffer | — | — | — | 20 μl | 20 μl |

Plasma samples were adjusted to bring 60014 of total protein in the reaction. The plates were incubated for 20 min at 37° C., and the absorbance was read at 450 nm. The SOD activity (SODA) was measured as follow and calculated using the standard curve:

SODA={[(A blank 1−A blank 3)−(A sample−A blank 2)]/(A blank−1A blank 3)}*100

### Plasma GPx Activity

The activity of the Glutathione peroxidase was determined using the GPx determination KIT (abcamab102530). Briefly, 40 μL of the colorimetric reaction mix was added to 100 μL of standards and 10 μL of cumene hydroperoxide was added to the standards and to the 50 μL of samples (Plasma samples were adjusted to bring 600 μg of total protein in the reaction). The absorbance at 340 nm was measured (A1), and after 5 min at 25° C., a second measurement was done (A2).

The GPx activity was calculated as follow:

$$1 \text{)} Δ A \_ 340 nm = ( ( Sample A 1 - Sample A 2 ) - \\ ( Reagent control A 1 - Reagent control A 2 ) )$$

$$2 \text{)} B = \frac{( Δ A \_ 340 nm - intercept}{Slope )}$$

$$3 \text{)} GPx Activity = \frac{( B )}{( T 2 - T 1 ) * V )} * D$$

Where:

B=NADPH amount that was decreased between T1 and T2 (in nmol).

T1=Time of the first reading (A1) (minutes).

T2=Time of second reading (A2) (minutes).

V=Pretreated sample volume added into the reaction well (mL).

D=Sample dilution factor.

### Statistical Analysis

ANOVA was performed for plasmatic parameters and student test was calculated to evaluate the significance of the differences observed in gene expression.

### Results

### Plasmatic Parameters

Results of the plasmatic parameters (SOD, CAT and GPx, OS) at day 0, 14 and 42 can be summarized as follows:
At day 14, the plasmatic activity of the antioxidant enzymes SOD, CAT and GPx were higher than at day 0.
For the GPx activity, at day 42, the addition of the enzymes in the diet showed a significant increase in group receiving either the CAT alone or in combination with the SOD.
At day 0, the reactive oxygen metabolites (d-ROM) and the 051 were higher than at day 14 and 42, reflecting the level of response to the stress generated in the early weaning period of the animals, and indicating a higher sensitivity of this biomarker in acute stress compared to the activity of SOD and CAT.
Interestingly, the d-ROM showed an increase in animals supplemented with CAT or CAT+SOD in both time point.
At day 14, the plasmatic antioxidant capacity showed a significant difference between experimental groups (p<0.0001) with an increase in group supplemented with CAT or SOD+CAT.

### Gene Expression Targeting Oxidative Stress

Results of the gene expression analysis are shown in Table 16 (Gene expression fold change of oxidative stress related genes in duodenum at day 14), Table 17 (Gene expression fold change of oxidative stress related genes in ileum at day 42) and can be summarized as follows:
In the group supplemented with both enzymes (SOD+CAT), a downregulation of the genes that encode NQO1 (protecting the cells against OS) and the anti-oxidant enzyme GSTP1 (p=0.07 and p=0.005 respectively) was observed suggesting that diet supplementation with a combination of SOD and CAT decreases stress protein expressions at the beginning of small intestine of weaned piglets.
SCARA3 showed also a significant increase in the same group (p=0.003), avoiding accumulation of ROS.
In duodenum at day 42, only the gene that encode the antioxidant enzyme GPX1 gene showed a significant upregulation (p=0.019) in piglets supplemented with CAT alone compared to control group with a fold change of 1.34. Those results are in line with the GPx plasmatic activity found in the piglets after 42 days of supplementation.
Piglets supplemented with SOD alone showed a upregulation on the protecting protein a NQO1
Similarly to SOD supplementation alone, piglets supplemented with CAT alone showed an upregulation of NQO1. Besides that, piglets showed a upregulation on antioxidant enzyme GSTP1 and a downregulation of a redox marker NOX5.
Finally, piglets supplemented with SOD+CAT showed a downregulation on the genes NOX5, oxidative stress marker, and CAT, antioxidant enzyme.

TABLE 16
Gene expression fold change of oxidative
stress related genes in duodenum

| | Treatments | | | |
|---|---|---|---|---|
| | NC | NC + SOD | NC + CAT | NC + SOD + CAT |
| Gene # | A | D | F | H |
| Nqo1 | 1 | −1.25 | — | −1.55 |
| TXN | 1 | −1.27 | — | −1.23 |
| NoX5 | 1 | 1.26 | — | 1.25 |
| HSP70 | 1 | 1.27 | — | −1.33 |
| GPX1 | 1 | −1.08 | — | −1.11 |
| SCARA3 | 1 | 1.18 | — | 1.53 ** |
| GPX3 | 1 | 1.12 | — | −1.18 |
| GSTP1 | 1 | −1.18 | — | −1.60 ** |
| SOD3 | 1 | 1.17 | — | 1.37 * |
| CAT | 1 | −1.05 | — | −1.45 * |
| PRDX5 | 1 | −1.22 * | — | −1.42 ** |

* p < 0.05;
** p < 0.01

TABLE 17
Gene expression fold change of oxidative
stress related genes in ileum.

| | NC | NC + SOD | NC + CAT | NC + SOD + CAT |
|---|---|---|---|---|
| Gene # | A | D | F | H |
| Nqo1 | 1 | 2.00 | 1.68 | 1.40 |
| TXN | 1 | 1.23 | 1.48 | −1.13 |
| NoX5 | 1 | −1.05 | −1.65 | −1.64 |
| HSP70 | 1 | −1.57 | −1.74 | −1.33 |
| GPX1 | 1 | −1.40 | 1.31 | −1.24 |
| SCARA3 | 1 | 1.40 | −1.26 | −1.16 |
| GPX3 | 1 | 1.20 | 1.29 | −1.24 |
| GSTP1 | 1 | −1.02 | 1.56 | 1.11 |
| SOD3 | 1 | 1.25 | 1.11 | 1.05 |
| CAT | 1 | −1.01 | −1.20 | −1.64 |
| PRDX5 | 1 | 1.05 | 1.40 | −1.10 |

### CONCLUSION

Plasmatic antioxidant enzymes, specially SOD and CAT, showed a clear increase at day 14, independently of the treatment, indicating that the oxidative stress situation induced by piglet weaning is progressively corrected during the postweaning period. A significant increase in the plasmatic antioxidant activity in piglets supplements with CAT alone could be observed. At day 42, piglets supplemented with CAT showed a significant increase of GPx activity.

Gene expression showed significant modulation at day 14 and not at day 42. In duodenum at day 14, the supplementation of antioxidant enzymes (SOD+CAT) showed a downregulation of the genes that encode NQO1 (protecting the cells against OS) and the anti-oxidant enzyme GSTP1 (p=0.07 and p=0.005 respectively) suggesting that diet supplementation with a combination of SOD and CAT decreases stress protein expressions at the beginning of small intestine of weaned piglets. SCARA3 showed also a significant increase in the same group (p=0.003), avoiding accumulation of ROS.

At the duodenum, only the gene that encode the antioxidant enzyme GPX1 showed a significant upregulation in piglets supplemented with CAT alone compared to control group and those results are in agreement with the GPx plasmatic activity found at 42 days. And at the ileum level, supplementation of CAT and SOD alone showed a upregulation on the protecting protein NQO1 and piglets supplemented with SOD+CAT showed a downregulation on the genes NOX5, oxidative stress marker, and CAT, antioxidant enzyme. Those data provide evidence that diet supplementation with a SOD, CAT and the combination of both enzymes, potentially decrease oxidative stress status in weaned piglets.

### Example 5 Cloning and Expression of Superoxide Dismutase from Trichoderma reesei

### Strains

Escherichia coli Top-10 strain purchased from Invitrogen (Life Technologies, Carlsbad, Calif., USA) was used to propagate our expression vectors encoding for lysozyme polypeptides. Aspergillus oryzae strain MT3568 was used for heterologous expression of the lysozyme polypeptide encoding sequences. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene.

### Media

DAP2C-1 medium was composed of 0.5 g yeast extract, 30 g Maltodextrin, 11 g magnesium sulphate heptahydrate, 1 g dipotassium phosphate, 2 g citric acid monohydrate, 5.2 g potassium phosphate tribasic monohydrate, 1 mL Dowfax 63N10 (antifoaming agent), 2.5 g calcium carbonate, supplemented with 1 mL KU6 metal solution, and deionised water to 1000 mL.

KU6 metal solution was composed of 6.8 g ZnCl₂, 2.5 g CuSO₄.5H₂O, 0.13 g NiCl₂, 13.9 g FeSO₄.7H₂O, 8.45 g MnSO₄.H₂O, 3 g C₆H₈O₇.H₂O, and deionised water to 1000 mL. YP 2% glucose medium was composed of 10 g yeast extract, 20 g Bacto-peptone, 20 g glucose, and deionised water to 1000 mL.

LB plates were composed of 10 g of Bacto-tryptone, 5 g of yeast extract, 10 g of sodium chloride, 15 g of Bacto-agar, and deionised water to 1000 mL.

LB medium was composed of 10 g of Bacto-tryptone, 5 g of yeast extract, and 10 g of sodium chloride, and deionised water to 1000 mL.

COVE-Sucrose-T plates were composed of 342 g of sucrose, 20 g of agar powder, 20 mL of COVE salt solution, and deionised water to 1000 mL. The medium was sterilized by autoclaving at 15 psi for 15 minutes (Bacteriological Analytical Manual, 8th Edition, Revision A, 1998). The medium was cooled to 60° C. and 10 mM acetamide, Triton X-100 (50 μL/500 mL) were added. COVE-N-Agar tubes were composed of 218 g Sorbitol, 10 g Dextrose, 2.02 g KNO₃, 25 g agar, 50 mL Cove salt solution, and deionised water up to 1000 mL.

COVE salt solution was composed of 2 6 g of MgSO₄.7H₂O, 26 g of KCl, 26 g of KH₂PO₄, 50 mL of COVE trace metal solution, and deionised water to 1000 mL.

COVE trace metal solution was composed of 0.04 g of Na₂B₄O₇.10H₂O, 0.4 g of CuSO₄.5H₂O, 1.2 g of FeSO₄.7H₂O, 0.7 g of MnSO₄.H₂O, 0.8 g of Na₂MoO₄.2H₂O, 10 g of ZnSO₄.7H₂O, and deionised water to 1000 mL.

### Cloning

Aspergillus niger MBin118 is disclosed in WO 2004/090155.

The SEQ ID NO 1 polypeptide coding sequence was cloned from Trichoderma reesei QM6a DNA by PCR.

Trichoderma reesei QM6a was cultivated in 100 ml of YP+2% glucose medium in 1000 ml Erlenmeyer shake flasks for 5 days at 20° C. Mycelia were harvested from the flasks by filtration of the medium through a Buchner vacuum funnel lined with MIRACLOTH® (EMD Millipore, Billerica, Mass., USA). Mycelia were frozen in liquid nitrogen and stored at −80C until further use. Genomic DNA was isolated using a DNEASY® Plant Maxi Kit (QIAGEN GMBH, Hilden Germany) according to the manufacturer's instructions.

Genomic sequence information was generated by Illumina MySeq (Illumina Inc., San Diego, Calif.). 5 rigs of the isolated Trichoderma reesei QM6a genomic DNA was used for library preparation and analysis according to the manufacturer's instructions. A 300 bp, paired end strategy was employed with a library insert size of 200-500 bp. The reads were subsequently fractionated to 25% followed by trimming (extracting longest sub-sequences having Phred-scores of 10 or more). These reads were assembled using ldba version 0.18. Contigs shorter than 200 bp were discarded. Genes were called using GeneMark.hmm ES version 2.3c and identification of the catalytic domain was made using “SOD_Cu” Hidden Markov Model provided by Pfam. A Swissprot entry of the identical sequence is also available: GORPL7 HYPJQ. The polypeptide coding sequence for the entire coding region was cloned from Trichoderma reesei QM6a genomic DNA by PCR using the primers (SEQ ID NO: A and SEQ ID NO: B) described below.

(SEQ ID NO: A)
5′-ACACAACTGGGGATCCACCATGCGGCCGTCTGGGTTCCT-3′
(SEQ ID NO: B)
5′-CTAGATCTCGAGAAGCTTTCACAGGGAGAAGAAGATGGC-3′

Bold letters represent Trichoderma harzianum enzyme coding sequence. Restriction sites are underlined. The sequence to the left of the restriction sites is homologous to the insertion sites of pDau109 (WO 2005/042735).

In-Fusion™ Advantage PCR Cloning Kit Cat. nr 639620

The amplification reaction (50 μl) was performed according to the manufacturer's instructions (Thermo Scientific) with the following final concentrations:

1× Phusion HC buffer
200 uM dNTP

### 2.0 mM MgCl₂

0.5 uM of each primer of SEQ ID NO: A+B
10 ng of Trichoderma harzianum O4 genomic DNA.

The PCR reaction was incubated in a DYAD® Dual-Block Thermal Cycler (BioRad, USA) programmed for 1 cycle at 98° C. for 2 minutes; 30 cycles each at 98° C. for 10 seconds and 72° C. for two minutes followed by 1 cycle at 72° C. for 6 minutes. Samples were cooled to 10° C. before removal and further processing.

Five μl of the PCR reaction were analyzed by 1% agarose gel electrophoresis using 40 mM Tris base, 20 mM sodium acetate, 1 mM disodium EDTA (TAE) buffer. A major band of about 1 kb was observed. The remaining PCR reaction was purified directly with an ILLUSTRA™ GFX™ PCR DNA and Gel Band Purification Kit (GE Healthcare, Piscataway, N.J., USA) according to the manufacturer's instructions.

Two μg of plasmid pDau109 was digested with Bam HI and Hind III and the digested plasmid was run on a 1% agarose gel using 50 mM Tris base-50 mM boric acid-1 mM disodium EDTA (TBE) buffer in order to remove the stuffer fragment from the restricted plasmid. The bands were visualized by the addition of SYBR® Safe DNA gel stain (Life Technologies Corporation, Grand Island, N.Y., USA) and use of a 470 nm wavelength transilluminator. The band corresponding to the restricted plasmid was excised and purified using an ILLUSTRA™ GFX™ PCR DNA and Gel Band Purification Kit. The plasmid was eluted into 10 mM Tris pH 8.0 and its concentration adjusted to 20 ng per μl. An IN-FUSION® PCR Cloning Kit (Clontech Laboratories, Inc., Mountain View, Calif., USA) was used to clone the 1450 bp PCR fragment into pDau109 digested with Bam HI and Hind III (20 ng). The IN-FUSION® total reaction volume was 10 μl. The IN-FUSION® total reaction volume was 10 μl. The IN-FUSION® reaction was transformed into FUSION-BLUE™ E. coli cells (Clontech Laboratories, Inc., Mountain View, Calif., USA) according to the manufacturer's protocol and plated onto LB agar plates supplemented with 50 μg of ampicillin per ml. After incubation overnight at 37° C., transformant colonies were observed growing under selection on the LB plates supplemented with 50 μg of ampicillin per mL.

Several colonies were selected for analysis by colony PCR using the pDau222 pDau109 vector primers described below. Four colonies were transferred from the LB plates supplemented with 50 μg of ampicillin per ml with a yellow inoculation pin (Nunc A/S, Denmark) to new LB plates supplemented with 50 μg of ampicillin per ml and incubated overnight at 37° C.

(SEQ ID NO: C)
Primer 8653: 5′-GCAAGGGATGCCATGCTTGG-3′
(SEQ ID NO: D)
Primer 8654: 5′-CATATAACCAATTGCCCTC-3′

Each of the three colonies were transferred directly into 200 μl PCR tubes composed of 5 μl of 2× Thermo Scientific Dream Tag™ PCR Master Mix (Thermo Fisher Scientific, Rockford, Ill., USA), 0.5 μl of primer 8653 (10 pm/μl), 0.5 μl of primer 8654 (10 pm/μl), and 4 μl of deionized water. Each colony PCR was incubated in a DYAD® Dual-Block Thermal Cycler programmed for 1 cycle at 94° C. for 60 seconds; 30 cycles each at 95° C. for 30 seconds, 60° C. for 45 seconds, 72° C. for 60 seconds, 68° C. for 10 minutes, and 10° C. for 10 minutes.

Four μl of each completed PCR reaction were submitted to 1% agarose gel electrophoresis using TAE buffer. All four E. coli transformants showed a PCR band of about 1 kb. Plasmid DNA was isolated from each of the four colonies using a QIAprep Spin Miniprep Kit (QIAGEN GMBH, Hilden Germany). The resulting plasmid DNA was sequenced with primers 8653 and 8654 using an Applied Biosystems Model 3730 Automated DNA Sequencer using version 3.1 BIG-DYE™ terminator chemistry (Applied Biosystems, Inc., Foster City, Calif., USA).

One plasmid was chosen for transforming Aspergillus oryzae MT3568. A. oryzae MT3568 is an amdS (acetamidase) disrupted gene derivative of Aspergillus oryzae JaL355 (WO 2002/40694) in which pyrG auxotrophy was restored by inactivating the A. oryzae amdS gene. Protoplasts of A. oryzae MT3568 were prepared according to the method described in European Patent, EP0238023, pages 14-15.

E. coli 190 containing the selected plasmid was grown overnight according to the manufacturer's instructions (Genomed) and the plasmid DNA was isolated using a Plasmid Midi Kit (Genomed JETquick kit, cat.nr. 400250, GENOMED GmbH, Germany) according to the manufacturer's instructions. The purified plasmid DNA was transformed into Aspergillus oryzae MT3568. A. oryzae MT3568 protoplasts were prepared according to the method of Christensen et al., 1988, Bio/Technology 6: 1419-1422. The selection plates consisted of COVE sucrose with +10 mM acetamide+15 mM CsCl+TRITON® X-100 (50 μl/500 ml). The plates were incubated at 37° C. Briefly, 8 uls of plasmid DNA representing 3 ugs of DNA was added to 100 uls MT3568 protoplasts. 250 ul of 60% PEG solution was added and the tubes were gently mixed and incubate at 37° for 30 minutes. The mix was added to 10 ml of pre-melted Cove top agarose (The top agarose melted and then the temperature equilibrated to 40 C in a warm water bath before being added to the protoplast mixture). The combined mixture was then plated on two Cove-sucrose selection petri plates with 10 mM acetamide. The plates are incubated at 37° C. for 4 days. Single Aspergillus transformed colonies were identified by growth on the selection Acetimide as a carbon source. Each of the four A. oryzae transformants were inoculated into 750 μl of YP medium supplemented with 2% glucose and also 750 μl of 2% maltodextrin and also DAP4C in 96 well deep plates and incubated at 37° C. stationary for 4 days. At same time the four transformants were restreaked on COVE-2 sucrose agar medium.

Culture broth from the Aspergillus oryzae transformants were then analyzed for production of the SEQ ID NO 1 polypeptide by SDS-PAGE using NUPAGE® 10% Bis-Tris SDS gels (Invitrogen, Carlsbad, Calif., USA) according to the manufacturer. Two bands at approximately 97 kDa and 45 kDa were observed for each of the Aspergillus oryzae transformants. One A. oryzae transformant producing the SEQ ID NO 1 polypeptide was selected and was cultivated in 1000 ml Erlenmeyer shake flasks containing 100 ml of DAP2C medium at 30° C. for 3 days with agitation at 150 rpm.

Using Aspergillus versicolor, Aspergillus deflectus, or Aspergillus egyptiacus, SEQ ID NO 2, 3, and 4 were similarly cloned and expressed.

### Example 6 Cloning and Expression of Fungal Superoxide Dismutase

Strains

Escherichia coli Top-10 strain purchased from TIANGEN (TIANGEN Biotech Co. Ltd., Beijing, China) was used to propagate our expression vector.

Aspergillus oryzae strain MT3568 (described in WO2015040159) was used for heterologous expression of the genes described in Table 1.

Aspergillus oryzae strain DAU785 (described in WO2018/113745) was used for heterologous expression of the genes described in the Table 2.

Media Dap4C medium was composed of 11 g MgSO₄.7H₂O, 1 g KH₂PO₄, 2.2 g Citric acid.H₂O, 20 g glucose, 10 g maltose, 5.2 g K₃PO₄H₂O, 0.5 g yeast extract, 1.25 g CaCO₃, 0.5 ml AMG Trace element solution and deionized water to 1 liter. After autoclave, 3.3 ml of 20% Lactic Acid (autoclaved) and 9.3 ml of 50% (NH₄)₂HPO₄ (sterile filtered) were added to every 400 ml above medium.

AMG Trace element solution was composed of 6.8 g ZnCl₂, 2.5 g CuSO₄.5H₂O, 0.24 g NiCl₂.5H₂O, 13.9 g FeSO₄.7H₂O, 13.6 g MnSO₄.5H₂O, 3 g Citric acid.H₂O, and deionised water to 1000 ml.

LB plates were composed of 10 g of Bacto-tryptone, 5 g of yeast extract, 10 g of sodium chloride, 15 g of Bacto-agar, and deionised water to 1000 ml.

LB medium was composed of 1 g of Bacto-tryptone, 5 g of yeast extract, and 10 g of sodium chloride, and deionised water to 1000 ml.

COVE sucrose plates were composed of 342 g of sucrose, 20 g of agar powder, 20 ml of COVE salt solution, and deionized water to 1 liter. The medium was sterilized by autoclaving at 15 psi for 15 minutes. For the transformation of MT3568, 10 mM acetamide was added, when the medium was cooled to 60° C.

COVE-2 plate/tube for isolation: 30 g/L sucrose, 20 ml/L COVE salt solution, 10 mM acetamide (only for the transformants of MT3568), 30 g/L noble agar (Difco, Cat #214220).

COVE salt solution was composed of 26 g of MgSO₄.7H₂O, 26 g of KCL, 26 g of KH₂PO₄, 50 ml of COVE trace metal solution, and deionised water to 1000 ml.

COVE trace metal solution was composed of 0.04 g of Na₂B₄O₇.10H₂O, 0.4 g of CuSO₄.5H₂O, 1.2 g of FeSO₄.7H₂O, 0.7 g of MnSO₄H₂O, 0.8 g of Na₂MoO₄.2H₂O, 10 g of ZnSO₄.7H₂O, and deionised water to 1000 ml.

The SOD genes derived from fungal strains isolated from environmental samples by standard microbiological isolation techniques. Strains were identified, and taxonomy was assigned based on DNA sequencing of the ITS

| SEQ ID | Donor scientific name |
|---|---|
| SEQ ID NO 9 | Westerdykella sp. AS85-2 |
| SEQ ID NO 10 | Aspergillus sp. XZ2669 |
| SEQ ID NO 11 | Preussia terricola |
| SEQ ID NO 12 | Kionochaeta sp. |
| SEQ ID NO 13 | Kionochaeta sp. |
| SEQ ID NO 16 | Preussia flanaganii |
| SEQ ID NO 17 | Cladobotryum sp. |
| SEQ ID NO 18 | Cladobotryum sp. |
| SEQ ID NO 19 | Westerdykella sp-46156 |
| SEQ ID NO 20 | Trichoderma hamatum |

| SEQ ID | Donor scientific name |
|---|---|
| SEQ ID NO 14 | Metapochonia bulbillosa |
| SEQ ID NO 15 | Xylomelasma sp. XZ0718 |
| SEQ ID NO 22 | Cephalotrichiella penicillata |
| SEQ ID NO 23 | Chaetomium megalocarpum |
| SEQ ID NO 24 | Chaetomium thermophilum var. thermophilum |
| SEQ ID NO 25 | Humicola hyalothermophila |
| SEQ ID NO 26 | Subramaniula anamorphosa |
| SEQ ID NO 28 | Trichoderma rossicum |
| SEQ ID NO 29 | Trichoderma lixii |
| SEQ ID NO 30 | Trichoderma sp-54723 |
| SEQ ID NO 31 | Aspergillus niveus |
| SEQ ID NO 32 | Aspergillus templicola |
| SEQ ID NO 33 | Pochonia chlamydosporia var. spinulospora |
| SEQ ID NO 34 | Trichoderma sp-44174 |
| SEQ ID NO 35 | Trichoderma rossicum |
| SEQ ID NO 36 | Trichoderma rossicum |
| SEQ ID NO 37 | Trichoderma sp-54723 |
| SEQ ID NO 39 | Trichoderma sp-44174 |
| SEQ ID NO 40 | Metapochonia suchlasporia |
| SEQ ID NO 41 | Metarhizium marquandii |
| SEQ ID NO 42 | Diaporthe nobilis |
| SEQ ID NO 43 | Tolypocladium sp. XZ2627 |
| SEQ ID NO 44 | Aspergillus japonicus |
| SEQ ID NO 45 | Metarhizium sp. XZ2431 |

Chromosomal DNA from individual strains was isolated by QIAamp Dneasy Kit (Qiagen, Hilden, Germany). 5 μg of chromosomal DNA were sent for full genome sequencing using Illumina technology. Genome sequencing, the subsequent assembly of reads and the gene discovery (i.e. annotation of gene functions) is known to the person skilled in the art and the service can be purchased commercially.

The genome sequences were analyzed for putative SOD from the PFAM database families PF00880, PF00881 and PF02777. This analysis identified genes encoding putative SOD, which were subsequently cloned and recombinantly expressed in Aspergillus oryzae.

The SOD genes were amplified by PCR respectively from above isolated genomic DNA. The purified PCR product was cloned into the previously digested pCaHj505 (for the genes listed in Table 1) or pDAU724 (for the genes listed in Table 2) by ligation with an IN-FUSION™ CF Dry-down Cloning Kit (Clontech Laboratories, Inc., Mountain View, Calif., USA) according to the manufacturer's instructions. The ligation mixture was used to transform E. coli TOP10 chemically competent cells (described in Strains). Correct colonies containing the corresponding SOD gene was selected and verified by DNA sequencing (by SinoGenoMax Company Limited, Beijing, China). The correct SOD containing colony was cultivated overnight in 3 ml of LB medium supplemented with 100 μg of ampicillin per ml. Plasmid DNA was purified using a Qiagen Spin Miniprep kit (Cat. 27106) (QIAGEN GmbH, Hilden, Germany) according to the manufacturer's instructions.

Protoplasts of Aspergillus oryzae MT3568 were prepared according to WO95/002043. Protoplasts of Aspergillus oryzae DAU785 were prepared according to WO2018/113745. 100 μl of protoplasts were mixed with 2.5-10 μg of the Aspergillus expression vector (above extracted plasmid) comprising the SOD gene and 250 μl of 60% PEG 4000, 10 mM CaCl₂, and 10 mM Tris-HCl pH7.5 and gently mixed. The mixture was incubated at 37° C. for 30 minutes and the protoplasts were spread onto COVE sucrose plates for selection. After incubation for 4-7 days at 37° C. spores of 4 transformants were inoculated into 3 ml of Dap4C medium.

After 3 days cultivation at 30° C., the culture broths were analyzed by SDS-PAGE using Novex® 4-20% Tris-Glycine Gel (Invitrogen Corporation, Carlsbad, Calif., USA) to identify the transformants producing the largest amount of recombinant SOD with respective estimated mature peptide size.

Spores of the best expressed transformant were spread on COVE-2 plates for re-isolation in order to isolate single colonies. Then a single colony was spread on a COVE-2 tube until sporulation.

Spores from the best expressed transformant were cultivated in 2400 ml of Dap4C medium in shake flasks during 3 days at a temperature of 30° C. under 80 rpm agitation. Culture broth was harvested by filtration using a 0.22 μm filter device. The filtered fermentation broth was used for enzyme characterization.

### Example 7 Cloning and Expression of Fungal Superoxide Dismutase

Strains

Escherichia coli Top-10 strain purchased from Invitrogen (Thermofisher Inc.) was used to propagate our expression vector.

Aspergillus oryzae strain MT3568 (described in WO2015040159) was used for heterologous expression of the genes described in Table 1.

Media

DAP4C medium is composed of 11 g MgSO₄.7H₂O, 1 g KH₂PO₄, 2.2 g Citric acid.H₂O, 20 g glucose, 10 g maltose, 5.2 g K₃PO₄.H₂O, 0.5 g yeast extract, 1.25 g CaCO₃, 0.5 ml AMG Trace element solution and deionized water to 1 liter. After autoclaving, 3.3 ml of 20% Lactic Acid (autoclaved) and 9.3 ml of 50% (NH₄)₂HPO₄ (sterile filtered) are added to every 400 ml of the above medium.

AMG Trace element solution is composed of 6.8 g ZnCl₂, 2.5 g CuSO₄.5H₂O, 0.24 g NiCl₂.5H₂O, 13.9 g FeSO₄.7H₂O, 13.6 g MnSO₄.5H₂O, 3 g Citric acid.H₂O, and deionised water to 1000 ml.

LB plates are composed of 10 g of Bacto-tryptone, 5 g of yeast extract, 10 g of sodium chloride, 15 g of Bacto-agar, and deionised water to 1000 ml.

LB medium is composed of 1 g of Bacto-tryptone, 5 g of yeast extract, and 10 g of sodium chloride, and deionised water to 1000 ml.

COVE sucrose plates are composed of 342 g of sucrose, 20 g of agar powder, 20 ml of COVE salt solution, and deionized water to 1 liter. The medium was sterilized by autoclaving. For the transformation of MT3568, 10 mM acetamide was added, when the medium was cooled to 60° C.

COVE-2 plate/tube for isolation if single transformants: 30 g/L sucrose, 20 ml/L COVE salt solution, 10 mM acetamide, 30 g/L noble agar (Difco, Cat #214220).

COVE salt solution is composed of 26 g of MgSO₄.7H₂O, 26 g of KCL, 26 g of KH₂PO₄, 50 ml of COVE trace metal solution, and deionised water to 1000 ml.

COVE trace metal solution is composed of 0.04 g of Na₂B₄O₇.10H₂O, 0.4 g of CuSO₄.5H₂O, 1.2 g of FeSO₄.7H₂O, 0.7 g of MnSO₄H₂O, 0.8 g of Na₂MoO₄.2H₂O, 10 g of ZnSO₄.7H₂O, and deionised water to 1000 ml.

Cloning, Expression and Fermentation of Fungal SOD Enzymes

The SOD genes were derived from fungal strains isolated from environmental samples using standard microbiological isolation techniques. The donor strains HEAL7057, HEAL7058 and HEAL7059 were identified, and taxonomy assigned based on the DNA sequencing of the ITS (Table 1). The donor fungal organism for HEAL7007 was Trichoderma reesei QM6a, a publicly available strain originally isolated from the Solomon Islands and is available in collections such as ATCC (ATCC 13631).

Chromosomal DNA from individual strains was isolated by QIAamp Dneasy Kit (Qiagen, Hilden, Germany). 5 μg of each genomic DNA sample were sent for full genome sequencing using Illumina technology. Genome sequencing, the subsequent assembly of reads and the gene discovery (i.e. annotation of gene functions) is known to persons skilled in the art and the service can also be purchased commercially.

The genome sequences were BLAST analyzed for putative SOD from the PFAM database families PF00080. This analysis identified genes encoding putative SOD, which were subsequently cloned and recombinantly expressed in Aspergillus oryzae.

The SOD genes were amplified by PCR respectively from above isolated genomic DNA. The purified PCR products were cloned into the previously digested pDau109 by ligation with an IN-FUSION™ CF Dry-down Cloning Kit (Clontech Laboratories, Inc., Mountain View, Calif., USA) according to the manufacturer's instructions. The plasmid pDAu109 and its use are described in (WO 2005/042735). The ligation mixture was used to transform E. coli TOP10 chemically competent cells (described in Strains). The cloned genes were sequenced and confirmed to be identical to the corresponding genes found in the genome sequences and transformed into the Aspergillus oryzae strain MT3568 (WO 11/057140) by the methods described in Christensen et al., 1988, Biotechnology 6, 1419-1422 and WO 04/032648. Transformants were selected during regeneration from protoplasts based on the ability, conferred by a selectable marker in the expression vector, to utilize acetamide as a nitrogen source, and were subsequently re-isolated under selection.

Production of the recombinant SOD peptides was evaluated by culturing the transformants in 96-well deep-well microtiter plates for 4 days at 30° C. in either a 0.25 ml or 0.75 ml volume of either or both YPG medium (WO 05/066338) or DAP-4C-1 medium (WO 12/103350) and monitoring peptide expression by SDS-PAGE. A single Aspergillus transformant was selected for each gene based on expression yields as evaluated in microtiter plate fermentation.

Spores of the best expressed transformant were spread on COVE-2 plates for re-isolation in order to isolate single colonies. Then a single colony was spread on a COVE-2 tube until sporulation.

For larger-scale production of the recombinant enzymes, and the Aspergillus transformants were cultured in 500 ml baffled flasks containing 150 ml of fermentation medium. Transformants expressing the SOD peptides were fermented in DAP-4C-1 medium (WO 12/103350). The cultures were shaken on a rotary table at 150 RPM at for 4 days, and the broth was subsequently separated from cellular material by passage through a 0.22 um filtration unit.

### Example 8 SOD Purification

The purification process for SOD was firstly applied with hydrophobic interaction chromatography, then if needed, ion exchange chromatography was applied. The difference for all the molecules was buffer type, pH, and salt concentration.

The culture supernatant of recombinant SOD was firstly added by ammonium sulfate with a final concentration of 2.2M and loaded into Phenyl Sepharose High Performance (GE Healthcare) equilibrated with 20 mM Tris-HCl at pH7.0 with 2.2M ammonium sulfate added. A gradient decrease of ammonium sulfate concentration from 2.2M to 0 was set up as elution condition. The elution fractions and flow-through faction were assayed by SDS-PAGE. The SOD activity was determined by the relative activity method of the SOD activity assay kit from Sigma (Cat No. 19160) described as below.

If the purity of enzyme purified by first step was not good and contains several bands, ion exchange chromatography will be applied for further purification. The fractions with SOD activity were pooled together and dialyzed with 20 mM Tris-HCl at pH7.0, then loaded into a Mono Q or Capto Q column (GE Healthcare) equilibrated with 20 mM Tris-HCl pH7.0. A gradient increase of NaCl concentration from 0 to 1M with 20 mM Tris-HCl at pH7.0 was set up as elution buffer. The elution fractions and flow-through fraction were assayed for SDS-PAGE and SOD activity.

The fractions with enzyme activity were pooled together and then diafiltrated with 20 mM Tris-HCl at pH7.0. The protein concentration was determined by Qubit® Protein Assay Kit (Invitrogen, cat Q33212).

### Example 9 SOD Activity Assay

SOD activity was determined using the relative active method of the SOD activity assay kit from Sigma (Cat No. 19160). 20 μl SOD with different dilution times by MQ water and 0.01% Triton X-100 was added into 200 μl WST solution (prepared as described for SOD activity assay kit), then 20 μl coupled enzyme solution from kit was added. The mixture was measured at 450 nm for 20 minutes at 37° C. (interval 40 sec, shake before first read). One slope could be created with mOD/min vs enzyme dosage, which could be set as standard curve for each SOD. Two commercial SODs from Sigma®, recombinant SOD bovine expressed in Escherichia coli (S9697) with 6108 U/mg EP and SOD from E. coli (S5639) with 10240 U/mg EP were set as specific activity reference.

Since the pH of WST working solution was about 10, the SOD activity at alkaline condition might be very low. The SOD activity assay was later adjusted by at pH7.8, which WST solution was replaced by Cytochrome C solution at pH7.8 (Cytochrome C, Sigma-Aldrich C7752). The Cytochrome C working solution contains 50 mM PBS at pH7.8, 0.1 mM EDTA, 0.01 mM CytoC, and 0.05 mM Xanthine (Sigma-Aldrich X0626). The absorbance of mixture was measured at 550 nm.

### Example 10 Gastric Stability Assay of SODs

Gastric stability was assayed with artificial gastric juice as stress condition. 20 μl SOD with appropriate dilution was added into 180 μl stress buffer (100 mM NaCl, 0.0013M HCl at pH3.0). The stress buffer with pepsin was prepared by adding 1.11 mg/ml pepsin (from porcine gastric mucosa, P7000, Sigma, 474 U/mg). The mixture was incubated at 37° C. in thermomixer for 0, 15, 30, 45, 60, 90, and 120 minutes separately. 10 μl sample extracted from the mixture was added into 90 μl activity buffer (K₂HPO₄/KH2PO4 mixed with final concentration of 100 mM PBS at pH7.0) as stop mixture. Then 20 μl stop mixture was added into 200 μl WST solution (or CytoC solution at pH7.8), and finally 20 μl coupled enzyme solution from kit was added to measure absorbance. The absorbance was measured at 450 nm for Sigma SOD kit (or 550 nm at pH 7.8) for 20 minutes at 37° C. (interval 40 sec, shake before first read). The activity at pH7.0 without stress condition was set as reference, and the residual activity at stress condition (pH3.0, pH3.0+pepsin) compared with reference was calculated as relative stability.

| | Gastric stability: | Gastric stability: |
|---|---|---|
| | pH 3, +pepsin 15 min - | pH 3, +pepsin 30 min - |
| ID | residual activity value % | residual activity value % |
| SOD, sigma | 35 | 26 |
| r E. coli | 43 | 43 |
| SEQ ID NO 1 | 98 | 99 |
| SEQ ID NO 10 | 81 | 83 |
| SEQ ID NO 11 | 70 | 75 |
| SEQ ID NO 12 | 88 | 88 |
| SEQ ID NO 13 | 92 | 89 |
| SEQ ID NO 14 | 100 | 100 |
| SEQ ID NO 15 | 100 | 100 |
| SEQ ID NO 16 | 90 | 92 |
| SEQ ID NO 17 | 100 | 100 |
| SEQ ID NO 18 | 100 | 100 |
| SEQ ID NO 19 | 52 | 53 |
| SEQ ID NO 20 | 98 | 97 |
| SEQ ID NO 22 | 84 | 85 |
| SEQ ID NO 23 | 45 | 45 |
| SEQ ID NO 24 | 57 | 58 |
| SEQ ID NO 25 | 62 | 62 |
| SEQ ID NO 26 | 97 | 97 |
| SEQ ID NO 27 | 78 | 81 |
| SEQ ID NO 28 | 128 | 122 |
| SEQ ID NO 29 | 144 | 126 |
| SEQ ID NO 30 | 124 | 125 |
| SEQ ID NO 31 | 126 | 140 |
| SEQ ID NO 32 | 90 | 90 |
| SEQ ID NO 33 | 105 | 90 |
| SEQ ID NO 34 | 120 | 127 |
| SEQ ID NO 35 | 101 | 113 |
| SEQ ID NO 36 | 96 | 96 |
| SEQ ID NO 37 | 100 | 105 |
| SEQ ID NO 38 | 98 | 92 |
| SEQ ID NO 39 | 104 | 116 |
| SEQ ID NO 40 | 83 | 88 |
| SEQ ID NO 41 | 118 | 116 |
| SEQ ID NO 42 | 98 | 99 |
| SEQ ID NO 43 | 109 | 107 |
| SEQ ID NO 44 | 104 | 116 |
| SEQ ID NO 45 | 104 | 111 |
| SEQ ID NO 46 | 79 | 77 |
| SEQ ID NO 47 | 41 | 42 |
| SEQ ID NO 48 | 61 | 60 |

As can be seen from the Table, the SODs of the invention are surprisingly gastric stable compared to conventional and commercial SODs.

### Example 11 Thermal Stability Assay of SODs

Thermal stability was quantified utilizing nano differential scanning fluorimetry (nDSF). To adjust pH, the samples were diluted 10-fold in a buffer cocktail containing 50 mM 2-(N-Morpholin)ethansulfonsyre (MES), 50 mM glycine, 50 mM acetic acid, pH 3-11. Sample dilutions were conducted using a Hamilton Microlab STAR Liquid Handling System in 96-well microtiter plates and transferred to 384-well microplates. The nDSF experiments were conducted utilizing either a Prometheus NT.48 or Prometheus NT.Plex with autosampler from Nanotemper. With the Prometheus NT.48, the samples were loaded manually using single capillaries (PR-0002), while samples were loaded in the Prometheus NT.Plex using capillary chips (PR-A0002). The experiments were conducted from 20 to 95° C. with a temperature gradient of 3.3° C./min. The transition temperatures (Tₘ-values) were obtained from peak values derived from the first-derivative of the signal trace (350/330 nm fluorescence ratio or 330 nm fluorescence) using PR. ThermControl software.

### Example 12 Catalase Cloning and Expression

### Strains

Escherichia coli Top-10 strain purchased from Invitrogen (Thermofisher Inc.) was used to propagate our expression vector.

Aspergillus oryzae strain MT3568 (described in WO2015040159) was used for heterologous expression of the genes described in Table 1.

### Media

DAP4C medium is composed of 11 g MgSO₄.7H₂O, 1 g KH₂PO₄, 2.2 g Citric acid.H₂O, 20 g glucose, 10 g maltose, 5.2 g K₃PO₄H₂O, 0.5 g yeast extract, 1.25 g CaCO₃, 0.5 ml AMG Trace element solution and deionized water to 1 liter. After autoclaving, 3.3 ml of 20% Lactic Acid (autoclaved) and 9.3 ml of 50% (NH₄)₂HPO₄ (sterile filtered) are added to every 400 ml of the above medium.

AMG Trace element solution is composed of 6.8 g ZnCl₂, 2.5 g CuSO₄.5H₂O, 0.24 g NiCl₂.5H₂O, 13.9 g FeSO₄.7H2O, 13.6 g MnSO₄.5H₂O, 3 g Citric acid.H₂O, and deionised water to 1000 ml.

LB plates are composed of 10 g of Bacto-tryptone, 5 g of yeast extract, 10 g of sodium chloride, 15 g of Bacto-agar, and deionised water to 1000 ml.

LB medium is composed of 1 g of Bacto-tryptone, 5 g of yeast extract, and 10 g of sodium chloride, and deionised water to 1000 ml.

COVE sucrose plates are composed of 342 g of sucrose, 20 g of agar powder, 20 ml of COVE salt solution, and deionized water to 1 liter. The medium was sterilized by autoclaving. For the transformation of MT3568, 10 mM acetamide was added, when the medium was cooled to 60° C.

COVE-2 plate/tube for isolation if single transformants: 30 g/L sucrose, 20 ml/L COVE salt solution, 10 mM acetamide, 30 g/L noble agar (Difco, Cat #214220).

COVE salt solution is composed of 26 g of MgSO₄.7H₂O, 26 g of KCL, 26 g of KH₂PO₄, 50 ml of COVE trace metal solution, and deionised water to 1000 ml.

COVE trace metal solution is composed of 0.04 g of Na₂B₄O₇.10H₂O, 0.4 g of CuSO₄.5H₂O, 1.2 g of FeSO₄.7H₂O, 0.7 g of MnSO₄H₂O, 0.8 g of Na₂MoO₄.2H₂O, 10 g of ZnSO₄.7H₂O, and deionised water to 1000 ml.

### Example 12: Cloning, Expression and Fermentation of Fungal Catalase Enzymes

The catalase genes were derived from fungal strains isolated from environmental samples using standard microbiological isolation techniques. The donor strains HEAL7001, was identified, and taxonomy assigned based on the DNA sequencing of the ITS (Table 1). The donor fungal organism for HEAL7060 was Curvularia verruculosa, a publicly available strain originally isolated from a grass inflorescence in The Gambian Republic, Africa. The strain was originally collected in 1966: Curvularia verruculosa Tandon & Bilgrami ex M. B. Ellis, Mycological Papers 106: 20 (1966).

Chromosomal DNA from individual strains was isolated by QIAamp Dneasy Kit (Qiagen, Hilden, Germany). 5 μg of each genomic DNA sample were sent for full genome sequencing using Illumina technology. Genome sequencing, the subsequent assembly of reads and the gene discovery (i.e. annotation of gene functions) is known to persons skilled in the art and the service can also be purchased commercially.

The genome sequences were BLAST analyzed for putative catalase from the PFAM database families PF00199 and PF18011. This analysis identified genes encoding putative catalases, which were subsequently cloned and recombinantly expressed in Aspergillus oryzae.

The catalase genes were amplified by PCR respectively from above isolated genomic DNA. The purified PCR products were cloned into the previously digested pDau109 by ligation with an IN-FUSION™ CF Dry-down Cloning Kit (Clontech Laboratories, Inc., Mountain View, Calif., USA) according to the manufacturer's instructions. The plasmid pDAu109 and its use are described in (WO 2005/042735). The ligation mixture was used to transform E. coli TOP10 chemically competent cells (described in Strains). The cloned genes were sequenced and confirmed to be identical to the corresponding genes found in the genome sequences and transformed into the Aspergillus oryzae strain MT3568 (WO 11/057140) by the methods described in Christensen et al., 1988, Biotechnology 6, 1419-1422 and WO 04/032648. Transformants were selected during regeneration from protoplasts based on the ability, conferred by a selectable marker in the expression vector, to utilize acetamide as a nitrogen source, and were subsequently re-isolated under selection.

Production of the recombinant catalase peptides was evaluated by culturing the transformants in 96-well deep-well microtiter plates for 4 days at 30° C. in either a 0.25 ml or 0.75 ml volume of either or both YPG medium (WO 05/066338) or DAP-4C-1 medium (WO 12/103350) and monitoring peptide expression by SDS-PAGE. A single Aspergillus transformant was selected for each gene based on expression yields as evaluated in microtiter plate fermentation.

Spores of the best expressed transformant were spread on COVE-2 plates for re-isolation in order to isolate single colonies. Then a single colony was spread on a COVE-2 tube until sporulation.

For larger-scale production of the recombinant enzymes, and the Aspergillus transformants were cultured in 500 ml baffled flasks containing 150 ml of fermentation medium. Transformants expressing the catalase peptides were fermented in DAP-4C-1 medium (WO 12/103350). The cultures were shaken on a rotary table at 150 RPM at for 4 days, and the broth was subsequently separated from cellular material by passage through a 0.22 um filtration unit.

### Example 13: Catalase Activity Assay

The catalase from Bovine Liver (Sigma®, Enzyme Commission (EC) Number: 1.11.1.6, CAS Number: 9001-05-2, Molecular weight: 250 kDa) has an activity of 3524 U/mg EP. Catalase activity was determined by H₂O₂ reduction detected at 240 nm. Firstly, catalase was diluted with different dilution times by MQ water and 0.01% Triton X-100. 10 μl enzyme sample, 90 μl activity buffer (K₂HPO₄/KH₂PO₄ mixed with final concentration of 100 mM PBS at pH7.0) were added into 50 μl 0.2% H₂O₂ solution (30% H₂O₂ was diluted to 0.2% in activity buffer). The mixture was measured at 240 nm for 10 minutes at room temperature (interval 34 sec, shake before first read). The commercial catalase from Sigma®, catalase from bovine liver (C1345), was set as reference. This allows for the selection of the suitable enzyme dosage.

| | Mature | Full length | Relative |
|---|---|---|---|
| | polypeptide | polypeptide | Activity |
| Source | SEQ ID NO | SEQ ID NO | U/mg EP |
| Talaromyces | SEQ ID NO | SEQ ID NO | 1667 |
| stipitatus | 105 | 153 | |
| Penicillium | SEQ ID NO | SEQ ID NO | 8265 |
| emersonii | 108 | 156 | |
| Penicillium | SEQ ID NO | SEQ ID NO | 30953 |
| oxalicum | 116 | 164 | |
| Thermoascus | SEQ ID NO | SEQ ID NO | 10919 |
| crustaceus | 118 | 166 | |
| Thermothelomyces | SEQ ID NO | SEQ ID NO | 18314 |
| thermophilus | 112 | 160 | |
| Curvularia | SEQ ID NO | SEQ ID NO | 20836 |
| verruculosa | 113 | 161 | |
| Aspergillus oryzae | SEQ ID NO | SEQ ID NO | 43975 |
| (NN051131) | 115 | 163 | |
| Aspergillus | SEQ ID NO | SEQ ID NO | 52327 |
| lentulus | 104 | 152 | |
| Aspergillus | SEQ ID NO | SEQ ID NO | 51648 |
| fumigatus | 111 | 159 | |
| Neurospora crassa | SEQ ID NO | SEQ ID NO | 8651 |
| | 123 | 171 | |
| Neurospora crassa | SEQ ID NO | SEQ ID NO | 51184 |
| | 121 | 169 | |
| Malbranchea | SEQ ID NO | SEQ ID NO | 82637 |
| cinnamomea | 106 | 154 | |
| Humicola | SEQ ID NO | SEQ ID NO | 56454 |
| hyalothermophila | 117 | 165 | |
| Thielavia | SEQ ID NO | SEQ ID NO | 5273 |
| australiensis | 119 | 167 | |
| Thermomucor | SEQ ID NO | SEQ ID NO | 7750 |
| indicae-seudaticae | 110 | 158 | |
| Crassicarpon | SEQ ID NO | SEQ ID NO | 39459 |
| thermophilum | 107 | 155 | |

### Example 14: Gastric Stability Assay for Catalase

Gastric stability was assayed with artificial gastric juice as stress condition. 100 catalase with appropriate dilution was added into 900 stress buffer (100 mM NaCl, 0.0013M HCl at pH3.0). The stress buffer with pepsin was prepared by adding 1.11 mg/ml pepsin (from porcine gastric mucosa, P7000, Sigma, 474 U/mg) as pH3+pepsin buffer, and also was incubated with 10 ul catalase. The mixture was incubated at 37° C. in thermomixer for 0, 30, 60 and 90 minutes separately. 10 μl sample extracted from the mixture was added into 90 μl activity buffer as stop mixture. Then 100 μl stop mixture was added with 50 μl 0.2% H₂O₂ solution to measure absorbance. The absorbance was measured at 240 nm for 10 minutes at room temperature (interval 34 sec, shake before first read). One slop could be calculated by OD vs min, which presents the activity. The activity at pH7.0 without stress condition was set as reference, and the residual activity at stress condition (pH3.0 or pH3.0+pepsin) compared with reference was calculated as relative stability.

| | Gastric stability: pH 3, +pepsin |
|---|---|
| Catalase SEQ ID NO | 30 min - residual value % |
| SEQ ID NO 109 | 55 |
| SEQ ID NO 126 | 65 |
| SEQ ID NO 7 | 85 |
| Terminox Ultra ™ (SEQ ID NO 250) | 95 |
| SEQ ID NO 105 | 97 |
| SEQ ID NO 108 | 85 |
| SEQ ID NO 116 | 98 |
| SEQ ID NO 105 | 81 |
| SEQ ID NO 112 | 97 |
| SEQ ID NO 115 | 77 |
| SEQ ID NO 104 | 82 |
| SEQ ID NO 111 | 46 |
| SEQ ID NO 117 | 97 |
| SEQ ID NO 107 | 100 |
| SEQ ID NO 110 | 100 |
| SEQ ID NO 123 | 100 |
| Catalase ™ (SEQ ID NO 7) | 39 |

1. An animal feed additive comprising a polypeptide of fungal origin having superoxide dismutase activity.

2. The animal feed additive according to claim 1 further comprising a catalase.

3. The animal feed additive according to claim 1 comprising an enzyme component, wherein the enzyme component comprises all of the enzymes of the additive and the enzyme component consists of a superoxide dismutase of fungal origin and optionally of a catalase.

4. An animal feed additive comprising at least one and no more than two enzyme classes, wherein the at least one enzyme class is a superoxide dismutase of fungal origin and the optional second enzyme class is a catalase.

5. The animal feed additive of claim 1, wherein the polypeptide having superoxide dismutase activity is selected from the group consisting of:
a. a polypeptide having at least 75% sequence identity to SEQ ID NO: 1;
b. a polypeptide having at least 75% sequence identity to SEQ ID NO: 2;
c. a polypeptide having at least 75% sequence identity to SEQ ID NO: 3;
d. a polypeptide having at least 75% sequence identity to SEQ ID NO: 4;
e. a polypeptide having at least 75% sequence identity to SEQ ID NO: 9;
f. a polypeptide having at least 75% sequence identity to SEQ ID NO: 10;
g. a polypeptide having at least 75% sequence identity to SEQ ID NO: 11;
h. a polypeptide having at least 75% sequence identity to SEQ ID NO: 12;
i. a polypeptide having at least 75% sequence identity to SEQ ID NO: 13;
j. a polypeptide having at least 75% sequence identity to SEQ ID NO: 14;
k. a polypeptide having at least 75% sequence identity to SEQ ID NO: 15;
l. a polypeptide having at least 75% sequence identity to SEQ ID NO: 16;
m. a polypeptide having at least 75% sequence identity to SEQ ID NO: 17;
n. a polypeptide having at least 75% sequence identity to SEQ ID NO: 19;
o. a polypeptide having at least 75% sequence identity to SEQ ID NO: 20;
p. a polypeptide having at least 75% sequence identity to SEQ ID NO: 21;
q. a polypeptide having at least 75% sequence identity to SEQ ID NO: 22;
r. a polypeptide having at least 75% sequence identity to SEQ ID NO: 23;
s. a polypeptide having at least 75% sequence identity to SEQ ID NO: 24;
t. a polypeptide having at least 75% sequence identity to SEQ ID NO: 25;
u. a polypeptide having at least 75% sequence identity to SEQ ID NO: 26;
v. a polypeptide having at least 75% sequence identity to SEQ ID NO: 27;
w. a polypeptide having at least 75% sequence identity to SEQ ID NO: 28;
x. a polypeptide having at least 75% sequence identity to SEQ ID NO: 29;
y. a polypeptide having at least 75% sequence identity to SEQ ID NO: 30;
z. a polypeptide having at least 75% sequence identity to SEQ ID NO: 31;
aa. a polypeptide having at least 75% sequence identity to SEQ ID NO: 32;
bb. a polypeptide having at least 75% sequence identity to SEQ ID NO: 33;
cc. a polypeptide having at least 75% sequence identity to SEQ ID NO: 34;
dd. a polypeptide having at least 75% sequence identity to SEQ ID NO: 36;
ee. a polypeptide having at least 75% sequence identity to SEQ ID NO: 38;
ff. a polypeptide having at least 75% sequence identity to SEQ ID NO: 39;
gg. a polypeptide having at least 75% sequence identity to SEQ ID NO: 40;
hh. a polypeptide having at least 75% sequence identity to SEQ ID NO: 41;
ii. a polypeptide having at least 75% sequence identity to SEQ ID NO: 42;
jj. a polypeptide having at least 75% sequence identity to SEQ ID NO: 43;
kk. a polypeptide having at least 75% sequence identity to SEQ ID NO: 44;
ll. a polypeptide having at least 75% sequence identity to SEQ ID NO: 45;
mm. a polypeptide having at least 75% sequence identity to SEQ ID NO: 46;
nn. a polypeptide having at least 75% sequence identity to SEQ ID NO: 47; and
oo. a polypeptide having at least 75% sequence identity to SEQ ID NO: 48;
pp. a polypeptide having at least 75% sequence identity to SEQ ID NO:199;
qq. a polypeptide having at least 75% sequence identity to SEQ ID NO:200;
rr. a polypeptide having at least 75% sequence identity to SEQ ID NO: 201;
ss. a polypeptide having at least 75% sequence identity to SEQ ID NO:202;
tt. a polypeptide having at least 75% sequence identity to SEQ ID NO:203;
uu. a polypeptide having at least 75% sequence identity to SEQ ID NO:204;
vv. a polypeptide having at least 75% sequence identity to SEQ ID NO: 205;
ww. a polypeptide having at least 75% sequence identity to SEQ ID NO:206;
xx. a polypeptide having at least 75% sequence identity to SEQ ID NO:207;
yy. a polypeptide having at least 75% sequence identity to SEQ ID NO:208;
zz. a polypeptide having at least 75% sequence identity to SEQ ID NO:209;
aaa. a polypeptide having at least 75% sequence identity to SEQ ID NO:232;
bbb. a polypeptide having at least 75% sequence identity to SEQ ID NO:233;
ccc. a polypeptide having at least 75% sequence identity to SEQ ID NO:234;
ddd. a polypeptide having at least 75% sequence identity to SEQ ID NO:235;
eee. a polypeptide having at least 75% sequence identity to SEQ ID NO:236; and
fff. a polypeptide having at least 75% sequence identity to SEQ ID NO:237.

6-12. (canceled)

13. A method of improving one or more performance parameters in an animal comprising administering to the animal the animal feed additive of claim 1, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).

14. A method of improving or enhancing immune response and/or reducing inflammation and/or for the modulation of the gut flora in an animal comprising administering to the animal the animal feed additive of claim 1.

15. A method of reducing or eliminating the use of antibiotics administered to an animal feed, comprising administering to the animal the animal feed additive of claim 1.

16. The method of claim 13, wherein the animal has experienced heat stress, cold stress, nutritional stress and/or oxidative stress.
